SEQUENCE LISTING (24 sequences)

SEQ ID NO: 1 - protein, 152 aa, homo sapien
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 108 aa, homo sapien
DCDIEGKDGKQYESVLMVSIDQLLDSMKEIGSNCLNNEFNFFKRHICDANKVKGRKPAALGEAQPTKSLEENKSLKEQKKLNDLCFLKRLLQEIKTCWNKILMGTKEH

SEQ ID NO: 3 - protein, 134 aa, homo sapien
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQEENKSLKEQKKLNDLCFLKRLLQEIKTCWNKILMGTKEH

SEQ ID NO: 4 - protein, 90 aa, homo sapien
DCDIEGKDGKQYESVLMVSIDQLLDSMKEIGSNCLNNEFNFFKRHICDANKEENKSLKEQKKLNDLCFLKRLLQEIKTCWNKILMGTKEH

SEQ ID NO: 5 - protein, 123 aa, homo sapien
DCDIEGKDGKQYESVLMVSIDQLLDSMKEIGSNCLNNEFNFFKRHICDANKEGMFLFRAARKLRQFLKMNSTGDFDLHLLKVSEGTTILLKEQKKLNDLCFLKRLLQEIKTCWNKILMGTKEH

SEQ ID NO: 6 - protein, 134 aa, homo sapien; identical to SEQ ID NO: 3

SEQ ID NO: 7 - protein, 96 aa, homo sapien
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQH

SEQ ID NO: 8 - protein, 108 aa, homo sapien; identical to SEQ ID NO: 2

SEQ ID NO: 9 - protein, 108 aa, homo sapien
DCDIEGKDGKQYESVLMVSIDQLLDSMKEIGSNCLNNEFNFFKRHISDANKVKGRKPAALGEAQPTKSLEENKSLKEQKKLNDLCFLKRLLQEIKTCWNKILMGTKEH

SEQ ID NO: 10 - protein, 126 aa, homo sapien
MKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 11 - protein, 400 aa, homo sapien
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEGGGGSGGGGSGGGGSVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 12 - protein, 282 aa, homo sapien
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSMNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - protein, 399 aa, homo sapien
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 25 aa, homo sapien
MFHVSFRYIFGLPPLILVLLPVASS

SEQ ID NO: 15 - protein, 19 aa, homo sapien
MGWSCIILFLVATATGVHS

SEQ ID NO: 16 - protein, 6 aa, artificial sequence
  Synthetic sequence
HHHHHH

SEQ ID NO: 17 - DNA, 558 bp, homo sapien
gaattcccgccgccaccatgggctggtcctgcatcattctgtttctggtggctaccgccaccggcgtgcactctgactgtgatatcgaaggcaaggacggcaagcagtacgagtccgtgctgatggtgtccatcgaccagctgctggactccatgaaggaaatcggctccaactgcctgaacaacgagttcaacttcttcaagcggcacatctgcgacgccaacaaagaaggcatgttcctgttcagagccgccagaaagctgcggcagttcctgaagatgaactccaccggcgacttcgacctgcatctgctgaaagtgtctgagggcaccaccatcctgctgaactgtaccggccaagtgaagggcagaaagcctgctgctctgggcgaagcccagcctaccaagtctctggaagagaacaagtccctgaaagagcagaagaagctgaacgacctctgcttcctgaagcggctgctgcaagagatcaagacctgctggaacaagatcctgatgggcaccaaagagcaccatcaccatcatcaccactgataagctt

SEQ ID NO: 18 - DNA, 456 bp, homo sapien
gactgtgatatcgaaggcaaggacggcaagcagtacgagtccgtgctgatggtgtccatcgaccagctgctggactccatgaaggaaatcggctccaactgcctgaacaacgagttcaacttcttcaagcggcacatctgcgacgccaacaaagaaggcatgttcctgttcagagccgccagaaagctgcggcagttcctgaagatgaactccaccggcgacttcgacctgcatctgctgaaagtgtctgagggcaccaccatcctgctgaactgtaccggccaagtgaagggcagaaagcctgctgctctgggcgaagcccagcctaccaagtctctggaagagaacaagtccctgaaagagcagaagaagctgaacgacctctgcttcctgaagcggctgctgcaagagatcaagacctgctggaacaagatcctgatgggcaccaaagagcac

SEQ ID NO: 19 - DNA, 57 bp, artificial sequence
  Synthetic sequence
atgggctggtcctgcatcattctgtttctggtggctaccgccaccggcgtgcactct

SEQ ID NO: 20 - DNA, 18 bp, Artificial Sequence
  Synthetic sequence
catcaccatcatcaccac

SEQ ID NO: 21 - protein, 177 aa, homo sapien
MGWSCIILFLVATATGVHSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHHHHHHH

SEQ ID NO: 22 - DNA, 1281 bp, homo sapien
gaattcccgccgccaccatgggctggtcctgcatcattctgtttctggtggctaccgccaccggcgtgcactctgactgtgatatcgaaggcaaggacggcaagcagtacgagtccgtgctgatggtgtccatcgaccagctgctggactccatgaaggaaatcggctccaactgcctgaacaacgagttcaacttcttcaagcggcacatctgcgacgccaacaaagaaggcatgttcctgttcagagccgccagaaagctgcggcagttcctgaagatgaactccaccggcgacttcgacctgcatctgctgaaagtgtctgagggcaccaccatcctgctgaactgtaccggccaagtgaagggcagaaagcctgctgctctgggcgaagcccagcctaccaagtctctggaagagaacaagtccctgaaagagcagaagaagctgaacgacctctgcttcctgaagcggctgctgcaagagatcaagacctgctggaacaagatcctgatgggcaccaaagagcatggcggcggaggatctggcggaggtggaagcggaggcggtggatctgaacctaagtcctgcgacaagacccacacctgtcctccatgtcctgctccagaactgctcggcggaccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtctgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctggtcaagggcttctacccctccgatatcgccgtggaatgggagtctaatggccagcctgagaacaactacaagacaacccctcctgtgctggacagcgacggctcattcttcctgtactccaagctgacagtggacaagtccagatggcagcagggcaacgtgttctcctgctccgtgatgcacgaggccctgcacaatcactacacccagaagtccctgtctctgtcccctggcaagtgataagctt

SEQ ID NO: 23 - DNA, 1197 bp, homo sapien
gactgtgatatcgaaggcaaggacggcaagcagtacgagtccgtgctgatggtgtccatcgaccagctgctggactccatgaaggaaatcggctccaactgcctgaacaacgagttcaacttcttcaagcggcacatctgcgacgccaacaaagaaggcatgttcctgttcagagccgccagaaagctgcggcagttcctgaagatgaactccaccggcgacttcgacctgcatctgctgaaagtgtctgagggcaccaccatcctgctgaactgtaccggccaagtgaagggcagaaagcctgctgctctgggcgaagcccagcctaccaagtctctggaagagaacaagtccctgaaagagcagaagaagctgaacgacctctgcttcctgaagcggctgctgcaagagatcaagacctgctggaacaagatcctgatgggcaccaaagagcatggcggcggaggatctggcggaggtggaagcggaggcggtggatctgaacctaagtcctgcgacaagacccacacctgtcctccatgtcctgctccagaactgctcggcggaccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtctgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctggtcaagggcttctacccctccgatatcgccgtggaatgggagtctaatggccagcctgagaacaactacaagacaacccctcctgtgctggacagcgacggctcattcttcctgtactccaagctgacagtggacaagtccagatggcagcagggcaacgtgttctcctgctccgtgatgcacgaggccctgcacaatcactacacccagaagtccctgtctctgtcccctggcaag

SEQ ID NO: 24 - protein, 418 aa, homo sapien
MGWSCIILFLVATATGVHSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2021/0000921 A1
(43) Pub. Date: Jan. 7, 2021

(54) METHODS AND COMPOSITIONS FOR MODULATING THYMIC FUNCTION

(71) Applicant: FLAGSHIP PIONEERING INNOVATIONS V. INC., Cambridge, MA (US)

(72) Inventors: David Arthur Berry, Newton, MA (US); Alexander Nichols, Cambridge, MA (US); Douglas Ewen Cameron, Auburndale, MA (US); Brett Blackman, Bedford, MA (US); Chantal Kuhn, Jamaica Plain, MA (US)

(21) Appl. No.: 16/067,948

(22) PCT Filed: Jan. 11, 2017
(86) PCT No.: PCT/US17/13032

§ 371 Date: Jul. 3, 2018

Related U.S. Application Data
(60) Provisional application No. 62/277,301, filed on Jan. 11, 2016.

(51) Int. Cl. A61K 38/20; A61K 39/395; C07K 16/28; A61P 31/00; A61P 35/00; A61P 37/04

(52) U.S. Cl. CPC A61K 38/2046; A61K 39/39541; C07K 16/2818; A61K 2039/505; A61P 35/00; A61P 37/04; A61P 31/00

(57) ABSTRACT

The invention described herein features methods and compositions for modifying thymic function and/or activity in a subject (e.g., a human, e.g., a human patient in need thereof; or a non-human animal, e.g., a companion animal or an agricultural animal).

### BACKROUND OF THE INVENTION

The thymus is a specialized primary lymphoid organ of the immune system, responsible for the development, selection and maturation of T cells. The thymus provides an inductive environment for development of T cells from hematopoietic progenitor cells. In addition, thymic stromal cells allow for the selection of a functional and self-tolerant T cell repertoire. The thymus is largest and most active during the neonatal and pre-adolescent periods. By the early teens, the thymus begins to atrophy and thymic stroma is mostly replaced by adipose tissue.

### SUMMARY OF THE INVENTION

The invention features methods and compositions for modifying thymic function and/or activity in a subject (e.g., a human, e.g., a human patient in need thereof; or a non-human animal, e.g., a companion animal or an agricultural animal).

In one aspect, the invention features methods of modifying (e.g., increasing or decreasing) T-cell exhaustion in a subject in need thereof. The method includes administering to the subject an agent that modulates T cell function in combination with an inhibitor of immune checkpoint, and assessing one or more markers of T cell exhaustion in the subject.

In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF), FGF21 or a functional fragment or derivative thereof), Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BM P4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, GFG21 or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule.

In one embodiment, the agent decreases T cell exhaustion and is selected from: IL-7, CD127, soluble IL-7Ra, a CD127 activating monoclonal antibody, an anti-IL-7 antibody, IL-12, IL-15, anti-IL-15R, IL-22, IL-23, a functional fragment or derivative of any of the above molecules, or a combination thereof. In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In one embodiment, the marker of T cell exhaustion is one or more of (e.g., 2, 3, 4, 5, 6 or more of): PD-1, PD-L1, PD-L2, CTLA-4, CD39, B7-H2, B7-H3, B7-H4, B7-H6, B7.1, B7.2, LAG-3, CD244 (2B4), CD96, CD160, CD127, TIM-1, TIM-3, TIM-4, TIGIT, VISTA, ICOS, HVEM, BTLA, a KIR receptor, gp49B, CD47, SIRPalpha, CD48, A2aR, ILT-2, indoleamine-2,3-dioxygenase, or a combination thereof.

In some embodiments, the one or more markers of T cell exhaustion in the subject includes IFNg activity, TNFa activity, IL-2 activity, granzyme A activity, granzyme B activity, granzyme K activity, perforin activity, cell proliferation rate, tumor-specific cellular lysis, or a combination thereof.

In one embodiment, the one or more marker of T cell exhaustion includes one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the one or more markers of T cell exhaustion is assessed in a CAR-T cell of the subject.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, chemotherapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In other embodiments, the subject has not been diagnosed with cancer. In one embodiment, the subject has a chronic infection, e.g., an infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has progressive multifocal leukoencephalopathy (PML). In some embodiments, the subject has been treated with an anti-alpha(4)-integrin agent, such as an anti-alpha(4)-integrin monoclonal antibody (e.g., natalizumab).

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the assessing is typically performed after the administration but may also be performed before the first administration and/or during a course a treatment, e.g., after a first, second, third, fourth or later administration, or periodically over a course of treatment, e.g., once a month, or once every 3 months.

In some embodiments, the method also includes a step of selecting a subject exhibiting T cell exhaustion.

In one embodiment, the method also includes assessing the subject prior to treatment or first administration and using the results of the assessment to select a subject for treatment.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where decreasing T cell exhaustion are desired, the method includes stopping the administration if the marker of T cell exhaustion is not decreased at least 5%, 10%, 15%, 20%, 30%, 40%, 50% or more; or the method includes increasing the periodicity of administration if the marker of T cell exhaustion is not decreased at least 5%, 10%, 15%, 20% or more; or the method includes increasing the dose of one or both of the agent that modulates T cell function and the inhibitor of immune checkpoint if the marker of T cell exhaustion is not decreased at least 5%, 10%, 15%, 20% or more.

In one embodiment the agent that modulates T cell function and the inhibitor of immune checkpoint are administered at a dosage and frequency sufficient to modify (e.g., increase or decrease) T cell exhaustion in the subject. In certain embodiments, the one or more markers of T cell exhaustion in the subject is increased or decreased at least 1%, 2%, 5%, 10%, 15%, 20%, 25%, 30%, 35%, 40%, 50%, 60%, 70%, 80% or more, compared to before the administration. In certain embodiments, the one or more markers of T cell exhaustion in the subject is increased or decreased between 5-20%, between 5-50%, between 10-50%, between 20-80%, between 20-70%.

In another aspect, the invention features a method of increasing tumor infiltrating lymphocytes (TILs) in a subject who has a tumor. The method includes administering to a subject in need thereof an agent that modulates thymus or T-cell function in combination with an inhibitor of immune checkpoint, and assessing TILs in the subject.

In one embodiment, assessing TILs in the subject is comprises (a) performing a quantitative or qualitative determination of TILs within a tumor from a subject and/or (b) assessing the number of lymphocytes within a tumor in the subject. In certain embodiments, TILs are measured in a tumor biopsy from the subject.

In certain embodiments, the method results in expansion of a functional subset of TILs in the subject. In certain embodiments, an increase in TILs is coincident with a modification of T-cell exhaustion within the TILs in the subject.

In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BM P4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule.

In one embodiment, the agent decreases T cell exhaustion and is selected from: IL-7, CD127, soluble IL-7Rα, a CD127 activating monoclonal antibody, an anti-IL-7 antibody, IL-12, IL-15, anti-IL-15R, IL-22, IL-23, a functional fragment or derivative of any of the above molecules, or a combination thereof.

In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In other embodiments, the subject has not been diagnosed with cancer. In one embodiment, the subject has a chronic infection, e.g., an infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has progressive multifocal leukoencephalopathy (PML). In some embodiments, the subject has been treated with an anti-alpha(4)-integrin agent, such as an anti-alpha(4)-integrin monoclonal antibody (e.g., natalizumab).

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the assessing is typically performed after the administration but may also be performed before the first administration and/or during a course a treatment, e.g., after a first, second, third, fourth or later administration, or periodically over a course of treatment, e.g., once a month, or once every 3 months.

In some embodiments, the method also includes a step of selecting a subject exhibiting inadequate levels of TIL, e.g., as determined by a tumor biopsy.

In one embodiment, the method also includes assessing the subject prior to treatment or first administration and using the results of the assessment to select a subject for treatment.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where increasing TIL are desired, the method includes stopping the administration if the TIL are not increased at least 5%, 10%, 15%, 20%, 30%, 40%, 50% or more; or the method includes increasing the periodicity of administration if the TIL are not increased at least 5%, 10%, 15%, 20% or more; or the method includes increasing the dose of one or both of the agent that modulates T cell function and the inhibitor of immune checkpoint if the TIL are not increased at least 5%, 10%, 15%, 20% or more.

In one embodiment the agent that modulates T cell function and the inhibitor of immune checkpoint are administered at a dosage and frequency sufficient to increase TIL in the subject. In certain embodiments, the TIL are increased at least 1%, 2%, 5%, 10%, 15%, 20%, 25%, 30%, 35%, 40%, 50%, 60%, 70%, 80% or more, compared to before the administration. In certain embodiments, the TIL are increased between 5-20%, between 5-50%, between 10-50%, between 20-80%, between 20-70%.

In another aspect, the invention features a method of modulating, e.g., modulating T cell receptor repertoire diversity, or increasing T cell diversity in a subject. The method includes administering to a subject in need thereof an agent that modulates thymus or T-cell function in combination with an inhibitor of immune checkpoint, and assessing one or more markers of T cell diversity in the subject.

In one embodiment, TCR diversity is defined as the diversity of TCRs in a sampled immune population (from blood, or other tissue) that makes the T cell population responsive to a variety of immune stimulations such as infection, vaccination, and cancer; as described previously in the art (PMID: 25619506, PMID: 24510963). In one embodiment, the one or more markers of T cell diversity is the number of unique TCR alpha and/or beta subunits detected within a sample, and sequence variations thereof, as determined by TCR sequencing using methods known to the art. In another embodiment, the one or more markers of T cell diversity is the number of peaks in a TCR spectrogram, as generated by TCR spectratyping. In another embodiment, the one or more markers of T cell diversity is the number of TRECs detected in or present in a sample. In another embodiment, the one or more markers of T cell diversity is the number of unique TCR tetramers that bind to distinct T cells or T cell clonal populations within a sample. In some embodiments, the one or more markers of T cell diversity increases with respect to a similar measurement performed on the same subject or group of subjects. In some embodiments, the one or more markers of T cell diversity increases post-treatment relative to pre-treatment. In another embodiment, the one or more markers of T cell diversity increases with respect to a different group of subjects that have not been subject to treatment with the invention. Note that in some embodiments, the comparator subject group that has not been treated with the invention is afflicted with a neoplasm or chronic infection, whereas in other embodiments the comparator subject group is healthy and free of neoplasm or chronic infection.

In one embodiment, the one or more marker sof T cell diversity assessing comprises one or more of: assessing TCR rearrangement excision circles (TREC)s, T-cell spectratyping, tetramer staining, sequencing of at least one T-cell receptor subunit, anti-TCR antibody staining, flow cytometry, or a combination thereof.

In some embodiments, T cell diversity is assessed by assessing TCR repertoire diversity, which refers to the number of different T cell receptors (TCR) in a population of T cells; sources of diversity within TCR repertoire include unique alpha and beta TCR subunits, genetic differences stemming from V-J (TCR alpha) and V-D-J (TCR beta) recombination, as well as different terminal deoxynucleotidyl transferase (TdT) - introduced nucleotides at DNA junctions, resulting in sequence variation. (Murphy et al., Janeway's Immunobiology, 8th Edition, 2012). In one embodiment, TCR repertoire diversity is shifted in the subject upon treatment with a combination therapy described herein.

In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BM P4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule.

In one embodiment, the agent increases T cell diversity and is selected from: IL-7, CD127, soluble IL-7Rα, a CD127 activating monoclonal antibody, an anti-IL-7 antibody, IL-12, IL-15, anti-IL-15R, IL-22, IL-23, a functional fragment or derivative of any of the above molecules, or a combination thereof.

In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In other embodiments, the subject has not been diagnosed with cancer. In one embodiment, the subject has a chronic infection, e.g., an infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has progressive multifocal leukoencephalopathy (PML). In some embodiments, the subject has been treated with an anti-alpha(4)-integrin agent, such as an anti-alpha(4)-integrin monoclonal antibody (e.g., natalizumab).

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the assessing is typically performed after the administration but may also be performed before the first administration and/or during a course a treatment, e.g., after a first, second, third, fourth or later administration, or periodically over a course of treatment, e.g., once a month, or once every 3 months.

In some embodiments, the method also includes a step of selecting a subject exhibiting inadequate levels of T cell diversity.

In one embodiment, the method also includes assessing the subject prior to treatment or first administration and using the results of the assessment to select a subject for treatment.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where increasing T cell diversity is desired, the method includes stopping the administration if the T cell diversity is not increased at least 5%, 10%, 15%, 20%, 30%, 40%, 50% or more; or the method includes increasing the periodicity of administration if the T cell diversity is not increased at least 5%, 10%, 15%, 20% or more; or the method includes increasing the dose of one or both of the agent that modulates T cell function and the inhibitor of immune checkpoint if the T cell diversity is not increased at least 5%, 10%, 15%, 20% or more.

In one embodiment the agent that modulates T cell function and the inhibitor of immune checkpoint are administered at a dosage and frequency sufficient to increase T cell diversity in the subject. In certain embodiments, T cell diversity is increased at least 1%, 2%, 5%, 10%, 15%, 20%, 25%, 30%, 35%, 40%, 50%, 60%, 70%, 80% or more, compared to before the administration. In certain embodiments, T cell diversity is increased between 5-20%, between 5-50%, between 10-50%, between 20-80%, between 20-70%.

In another aspect, the invention features a method of increasing T-cell clonality in a subject in need thereof. The method includes administering to the subject (i) an agent that modulates thymus or T-cell function in combination with (ii) an inhibitor of immune checkpoint, and assessing one or more markers of T cell clonality in the subject.

In one embodiment, assessing a marker of T cell clonality includes assessing TCR rearrangement excision circles (TREC)s, performing T-cell spectratyping, performing tetramer staining, sequencing at least one T-cell receptor subunit, staining for anti-TCR antibody, performing flow cytometry, or a combination thereof.

In one embodiment, the method also includes assessing IFNg function or activity, TNFa function or activity, IL-2 function or activity, granzyme A function or activity, granzyme B function or activity, granzyme K function or activity, perforin function or activity, cell proliferation rate, tumor-specific cellular lysis, or a combination thereof.

In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BMP4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule. In one embodiment, the agent decreases T cell exhaustion and is selected from: IL-7, CD127, soluble IL-7Rα, a CD127 activating monoclonal antibody, an anti-IL-7 antibody, IL-12, IL-15, anti-IL-15R, IL-22, IL-23, a functional fragment or derivative of any of the above molecules, or a combination thereof.

In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In other embodiments, the subject has not been diagnosed with cancer.

In one embodiment, the subject has a chronic infection, e.g., an infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has progressive multifocal leukoencephalopathy (PML). In some embodiments, the subject has been treated with an anti-alpha(4)-integrin agent, such as an anti-alpha(4)-integrin monoclonal antibody (e.g., natalizumab).

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the assessing is typically performed after the administration but may also be performed before the first administration and/or during a course a treatment, e.g., after a first, second, third, fourth or later administration, or periodically over a course of treatment, e.g., once a month, or once every 3 months.

In some embodiments, the method also includes a step of selecting a subject exhibiting inadequate levels of T cell clonality, e.g., as determined by a tumor biopsy.

In one embodiment, the method also includes assessing the subject prior to treatment or first administration and using the results of the assessment to select a subject for treatment.

In one embodiment, the method also includes modifying the administering step (e.g., stopping the administration, increasing or decreasing the periodicity of administration, increasing or decreasing the dose of one or both of the agents that modulates T cell function and the inhibitor of immune checkpoint) based on the results of the assessment. For example, in embodiments where increasing T cell clonality is desired, the method includes stopping the administration if T cell clonality is not increased at least 5%, 10%, 15%, 20%, 30%, 40%, 50% or more; or the method includes increasing the periodicity of administration if T cell clonality is not increased at least 5%, 10%, 15%, 20% or more; or the method includes increasing the dose of one or both of the agent that modulates T cell function and the inhibitor of immune checkpoint if T cell clonality is not increased at least 5%, 10%, 15%, 20% or more.

In one embodiment the agent that modulates T cell function and the inhibitor of immune checkpoint are administered at a dosage and frequency sufficient to increase T cell clonality in the subject. In certain embodiments, T cell clonality is increased at least 1%, 2%, 5%, 10%, 15%, 20%, 25%, 30%, 35%, 40%, 50%, 60%, 70%, 80% or more, compared to before the administration. In certain embodiments, T cell clonality is increased between 5-20%, between 5-50%, between 10-50%, between 20-80%, between 20-70%.

In another aspect, the invention features a method of increasing thymocytes in a subject in need thereof. The method includes administering to the subject (i) an agent that modulates thymus or T-cell function in combination with (ii) an inhibitor of immune checkpoint, and assessing one or more markers of thymocytes in the subject.

In one embodiment, assessing one or more markers of thymocytes includes one or more of: assessing early thymic progenitors (ETP), e.g., CD4 and CD8 surface marker double negative 1 (DN1) thymocytes characterized by expression of surface markers CD44 and CD117 and lack of CD25; DN2 thymocytes characterized by expression of surface markers CD44, CD25, and intermediate expression of CD117; DN3 thymocytes characterized by expression of surface marker CD25, invariant chain T cell receptor beta (icTCRb) and low expression of CD44 and CD117; DN4 thymocytes characterized by expression of surface marker CD25 and icTCRb, and absence of CD44 and CD117 expression; CD4 and CD8 double positive (DP) TCR negative cells; pre-positive selection DP thymocytes expressing low levels of TCR, post-positive selection DP thymocytes expressing high levels of CD5, CD69, and TCR; CD8 single positive and CD4 single positive thymic T cells; or any combination thereof.

In one embodiment, the method also includes assessing gamma-delta T cells, natural killer T cells, or a combination thereof, in the tumor.

In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BM P4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule. In one embodiment, the agent decreases T cell exhaustion and is selected from: IL-7, CD127, soluble IL-7Rα, a CD127 activating monoclonal antibody, an anti-IL-7 antibody, IL-12, IL-15, anti-IL-15R, IL-22, IL-23, a functional fragment or derivative of any of the above molecules, or a combination thereof.

In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In other embodiments, the subject has not been diagnosed with cancer. In one embodiment, the subject has a chronic infection, e.g., an infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has progressive multifocal leukoencephalopathy (PML). In some embodiments, the subject has been treated with an anti-alpha(4)-integrin agent, such as an anti-alpha(4)-integrin monoclonal antibody (e.g., natalizumab).

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the assessing is typically performed after the administration but may also be performed before the first administration and/or during a course a treatment, e.g., after a first, second, third, fourth or later administration, or periodically over a course of treatment, e.g., once a month, or once every 3 months.

In some embodiments, the method also includes a step of selecting a subject exhibiting inadequate levels of TIL, e.g., as determined by a tumor biopsy.

In one embodiment, the method also includes assessing the subject prior to treatment or first administration and using the results of the assessment to select a subject for treatment.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where increasing thymocytes, e.g., one or more markers of thymocytes, are desired, the method includes stopping the administration if the thymocytes, e.g., one or more markers of thymocytes, are not increased at least 5%, 10%, 15%, 20%, 30%, 40%, 50% or more; or the method includes increasing the periodicity of administration if the thymocytes, e.g., one or more markers of thymocytes, are not increased at least 5%, 10%, 15%, 20% or more; or the method includes increasing the dose of one or both of the agent that modulates T cell function and the inhibitor of immune checkpoint if the thymocytes, e.g., one or more markers of thymocytes, are not increased at least 5%, 10%, 15%, 20% or more.

In one embodiment the agent that modulates T cell function and the inhibitor of immune checkpoint are administered at a dosage and frequency sufficient to increase thymocytes, e.g., one or more markers of thymocytes, in the subject. In certain embodiments, the thymocytes, e.g., one or more markers of thymocytes, are increased at least 1%, 2%, 5%, 10%, 15%, 20%, 25%, 30%, 35%, 40%, 50%, 60%, 70%, 80% or more, compared to before the administration. In certain embodiments, thymocytes, e.g., one or more markers of thymocytes, are increased between 5-20%, between 5-50%, between 10-50%, between 20-80%, between 20-70%.

In another aspect, the invention features a method of increasing thymus size or preventing reduction of thymic size in a subject in need thereof. The method includes administering to the subject (i) an agent that modulates thymus or T-cell function in combination with (ii) an inhibitor of immune checkpoint, and (b) assessing thymus size in the subject.

In one embodiment, the assessing includes performing an MRI or CT scan on the subject. In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BM P4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule. In one embodiment, the agent decreases T cell exhaustion and is selected from: IL-7, CD127, soluble IL-7Rα, a CD127 activating monoclonal antibody, an anti-IL-7 antibody, IL-12, IL-15, anti-IL-15R, IL-22, IL-23, a functional fragment or derivative of any of the above molecules, or a combination thereof.

In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis. In on embodiment, the method prevents or reduces a reduction of thymic size associated with cancer treatment.

In other embodiments, the subject has not been diagnosed with cancer. In one embodiment, the subject has a chronic infection, e.g., an infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has progressive multifocal leukoencephalopathy (PML). In some embodiments, the subject has been treated with an anti-alpha(4)-integrin agent, such as an anti-alpha(4)-integrin monoclonal antibody (e.g., natalizumab).

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the assessing is typically performed after the administration but may also be performed before the first administration and/or during a course a treatment, e.g., after a first, second, third, fourth or later administration, or periodically over a course of treatment, e.g., once a month, or once every 3 months.

In some embodiments, the method also includes a step of selecting a subject exhibiting inadequate thymus size.

In one embodiment, the method also includes assessing the subject prior to treatment or first administration and using the results of the assessment to select a subject for treatment.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where increasing thymus size is desired, the method includes stopping the administration if thymus size is not increased at least 5%, 10%, 15%, 20%, 30%, 40%, 50% or more; or the method includes increasing the periodicity of administration if thymus size is not increased at least 5%, 10%, 15%, 20% or more; or the method includes increasing the dose of one or both of the agent that modulates T cell function and the inhibitor of immune checkpoint if thymus size is not increased at least 5%, 10%, 15%, 20% or more.

In one embodiment the agent that modulates T cell function and the inhibitor of immune checkpoint are administered at a dosage and frequency sufficient to increase thymus size in the subject. In certain embodiments, thymus size is increased at least 1%, 2%, 5%, 10%, 15%, 20%, 25%, 30%, 35%, 40%, 50%, 60%, 70%, 80% or more, compared to before the administration. In certain embodiments, thymus size is increased between 5-20%, between 5-50%, between 10-50%, between 20-80%, between 20-70%.

In another aspect, the invention features a method of increasing thymic epithelial cells in a subject in need thereof. The method includes administering to the subject (i) an agent that modulates thymus or T-cell function in combination with (ii) an inhibitor of immune checkpoint, and assessing one or more markers associated with thymic epithelial cells in the subject.

In one embodiment, the one or more markers of thymic epithelial cells is selected from: K8, K5, FoxN1, Hoxa3, EpCAM, AIRE, FezF2, MHC I, MHC II, CD45, CD80, CD86, CD90, proteasome subunit 135t, CD11c, RANK, RANKL, BMP4, retinoic acid, Wnt, Shh, FGF, CCL25, CXCL12, SCF, and combinations thereof.

In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BMP4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule. In one embodiment, the agent decreases T cell exhaustion and is selected from: IL-7, CD127, soluble IL-7Ra, a CD127 activating monoclonal antibody, an anti-IL-7 antibody, IL-12, IL-15, anti-IL-15R, IL-22, IL-23, a functional fragment or derivative of any of the above molecules, or a combination thereof.

In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In other embodiments, the subject has not been diagnosed with cancer. In one embodiment, the subject has a chronic infection, e.g., an infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has progressive multifocal leukoencephalopathy (PML). In some embodiments, the subject has been treated with an anti-alpha(4)-integrin agent, such as an anti-alpha(4)-integrin monoclonal antibody (e.g., natalizumab).

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the assessing is typically performed after the administration but may also be performed before the first administration and/or during a course a treatment, e.g., after a first, second, third, fourth or later administration, or periodically over a course of treatment, e.g., once a month, or once every 3 months.

In some embodiments, the method also includes a step of selecting a subject exhibiting inadequate levels of thymic epithelial cells.

In one embodiment, the method also includes assessing the subject prior to treatment or first administration and using the results of the assessment to select a subject for treatment.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where increasing thymic epithelial cells, e.g., a marker of thymic epithelial cells, is desired, the method includes stopping the administration if the thymic epithelial cells, e.g., a marker of thymic epithelial cells, are not increased at least 5%, 10%, 15%, 20%, 30%, 40%, 50% or more; or the method includes increasing the periodicity of administration if the thymic epithelial cells, e.g., a marker of thymic epithelial cells, are not increased at least 5%, 10%, 15%, 20% or more; or the method includes increasing the dose of one or both of the agent that modulates T cell function and the inhibitor of immune checkpoint if thymic epithelial cells, e.g., a marker of thymic epithelial cells, are not increased at least 5%, 10%, 15%, 20% or more.

In one embodiment the agent that modulates T cell function and the inhibitor of immune checkpoint are administered at a dosage and frequency sufficient to increase thymic epithelial cells, e.g., a marker of thymic epithelial cells, in the subject. In certain embodiments, the thymic epithelial cells, e.g., a marker of thymic epithelial cells, are increased at least 1%, 2%, 5%, 10%, 15%, 20%, 25%, 30%, 35%, 40%, 50%, 60%, 70%, 80% or more, compared to before the administration. In certain embodiments, thymic epithelial cells, e.g., a marker of thymic epithelial cells, are increased between 5-20%, between 5-50%, between 10-50%, between 20-80%, between 20-70%.

In another aspect, the invention features a method of increasing thymic stromal cells in a subject in need thereof. The method includes administering to the subject (i) an agent that modulates thymus or T-cell function in combination with (ii) an inhibitor of immune checkpoint, and assessing one or more stromal cells (e.g., fibroblasts and/or endothelial cells) in the thymus subject.

In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BM P4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule. In one embodiment, the agent decreases T cell exhaustion and is selected from: IL-7, CD127, soluble IL-7Rα, a CD127 activating monoclonal antibody, an anti-IL-7 antibody, IL-12, IL-15, anti-IL-15R, IL-22, IL-23, a functional fragment or derivative of any of the above molecules, or a combination thereof.

In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In other embodiments, the subject has not been diagnosed with cancer. In one embodiment, the subject has a chronic infection, e.g., an infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has progressive multifocal leukoencephalopathy (PML). In some embodiments, the subject has been treated with an anti-alpha(4)-integrin agent, such as an anti-alpha(4)-integrin monoclonal antibody (e.g., natalizumab).

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the assessing is typically performed after the administration but may also be performed before the first administration and/or during a course a treatment, e.g., after a first, second, third, fourth or later administration, or periodically over a course of treatment, e.g., once a month, or once every 3 months.

In some embodiments, the method also includes a step of selecting a subject exhibiting inadequate levels of thymic stromal cells, e.g., one or more markers of thymic stromal cells.

In one embodiment, the method also includes assessing the subject prior to treatment or first administration and using the results of the assessment to select a subject for treatment.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where increasing thymic stromal cells, e.g., one or more markers of thymic stromal cells, is desired, the method includes stopping the administration if the thymic stromal cells, e.g., one or more markers of thymic stromal cells, are not increased at least 5%, 10%, 15%, 20%, 30%, 40%, 50% or more; or the method includes increasing the periodicity of administration if the thymic stromal cells, e.g., one or more markers of thymic stromal cells, are not increased at least 5%, 10%, 15%, 20% or more; or the method includes increasing the dose of one or both of the agent that modulates T cell function and the inhibitor of immune checkpoint if thymic stromal cells, e.g., one or more markers of thymic stromal cells, are not increased at least 5%, 10%, 15%, 20% or more.

In one embodiment the agent that modulates T cell function and the inhibitor of immune checkpoint are administered at a dosage and frequency sufficient to increase thymic stromal cells, e.g., one or more markers of thymic stromal cells, in the subject. In certain embodiments, thymic stromal cells, e.g., one or more markers of thymic stromal cells, are increased at least 1%, 2%, 5%, 10%, 15%, 20%, 25%, 30%, 35%, 40%, 50%, 60%, 70%, 80% or more, compared to before the administration. In certain embodiments, thymic stromal cells, e.g., one or more markers of thymic stromal cells, are increased between 5-20%, between 5-50%, between 10-50%, between 20-80%, between 20-70%.

In another aspect, the invention features a method of increasing persistence of a cell therapy in a subject, e.g., a cell therapy comprising administration of T cells, e.g., augmented self T cells, T cells genetically engineered to express a receptor, e.g., a CAR-T cell therapy. The method includes administering to the subject (i) an agent that modulates thymus or T-cell function in combination with (ii) an inhibitor of immune checkpoint and/or a preparation of T cells comprising a chimeric antigen receptor (a CAR-T cell therapy). In some embodiments, the cell therapy, e.g., CAR-T cells, have increased persistence or function in the subject relative to their persistence or function in the absence of the agent that modulates thymus or T-cell function and/or the inhibitor of immune checkpoint alone or in combination. In some embodiments, the method includes administering to the subject (i) an agent that modulates thymus or T-cell function in combination with (ii) an inhibitor of immune checkpoint and (a) a preparation of T cells comprising a chimeric antigen receptor (a CAR-T cell therapy).

In some embodiments, the cell therapy is CAR-T cell therapy. CAR-T persistence and/or function in a subject in need thereof. In some embodiments, the method includes assessing CAR-T persistence or function in the subject. By way of non-limiting example, CAR-T persistence can be assessed through the use of a highly sensitive PCR assay for the engineered transgene, as described in the art (e.g., PMID 23831595)

In some embodiments, the T cells are autologous to the subject. In other embodiments, the T cells are heterologous.

In some embodiments, the CAR of the CAR-T therapy targets CD19, CD22, or CD20. In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BM P4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule. In one embodiment, the agent increases CAR-T persistence and/or function and is selected from: IL-7, CD127, soluble IL-7Ra, a CD127 activating monoclonal antibody, an anti-IL-7 antibody, IL-12, IL-15, anti-IL-15R, IL-22, IL-23, a functional fragment or derivative of any of the above molecules, or a combination thereof. In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In some embodiments, the method increases CAR-T persistence or function in the subject. In one embodiment, the subject has been diagnosed with cancer, e.g., a hematological cancer or a solid cancer or tumor such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In other embodiments, the subject has not been diagnosed with cancer. In one embodiment, the subject has a chronic infection, e.g., an infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has progressive multifocal leukoencephalopathy (PML). In some embodiments, the subject has been treated with an anti-alpha(4)-integrin agent, such as an anti-alpha(4)-integrin monoclonal antibody (e.g., natalizumab).

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the assessing is typically performed after the administration but may also be performed before the first administration and/or during a course a treatment, e.g., after a first, second, third, fourth or later administration, or periodically over a course of treatment, e.g., once a month, or once every 3 months.

In some embodiments, the method also includes a step of selecting a subject exhibiting inadequate levels of CAR-T persistence or function.

In one embodiment, the method also includes assessing the subject prior to treatment or first administration and using the results of the assessment to select a subject for treatment.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where increasing CAR-T persistence or function is desired, the method includes stopping the administration if CAR-T persistence or function not increased at least 5%, 10%, 15%, 20%, 30%, 40%, 50% or more; or the method includes increasing the periodicity of administration if CAR-T persistence or function is not increased at least 5%, 10%, 15%, 20% or more; or the method includes increasing the dose of one or both of the agent that modulates T cell function and the inhibitor of immune checkpoint if CAR-T persistence or function is not increased at least 5%, 10%, 15%, 20% or more.

In one embodiment the agent that modulates T cell function and the inhibitor of immune checkpoint are administered at a dosage and frequency sufficient to increase CAR-T persistence or function in the subject. In certain embodiments, CAR-T persistence or function is increased at least 1%, 2%, 5%, 10%, 15%, 20%, 25%, 30%, 35%, 40%, 50%, 60%, 70%, 80% or more, compared to before the administration. In certain embodiments, CAR-T persistence or function is increased between 5-20%, between 5-50%, between 10-50%, between 20-80%, between 20-70%.

In another aspect, the invention features a method of inducing long lived immunity in a subject. The method includes administering to the subject (i) an agent that modulates thymus or T-cell function in combination with (ii) an inhibitor of immune checkpoint.

In one embodiment, the method includes assessing the subject for a marker of memory cells, e.g., one or both of: KLRG1+ CD127- (SLECS) and KLRG1- CD127+ cells (MPECS).

In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BMP4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule. In one embodiment, the agent increases memory cells, e.g., one or more markers of memory cells, and is selected from: IL-7, CD127, soluble IL-7Rα, a CD127 activating monoclonal antibody, an anti-IL-7 antibody, IL-12, IL-15, anti-IL-15R, IL-22, IL-23, a functional fragment or derivative of any of the above molecules, or a combination thereof. In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In other embodiments, the subject has not been diagnosed with cancer.

In one embodiment, the subject has a chronic infection, e.g., an infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has progressive multifocal leukoencephalopathy (PML). In some embodiments, the subject has been treated with an anti-alpha(4)-integrin agent, such as an anti-alpha(4)-integrin monoclonal antibody (e.g., natalizumab).

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the assessing is typically performed after the administration but may also be performed before the first administration and/or during a course a treatment, e.g., after a first, second, third, fourth or later administration, or periodically over a course of treatment, e.g., once a month, or once every 3 months.

In some embodiments, the method also includes a step of selecting a subject exhibiting inadequate levels of memory cells, e.g., one or more markers of memory cells.

In one embodiment, the method also includes assessing the subject prior to treatment or first administration and using the results of the assessment to select a subject for treatment.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where increasing memory cells, e.g., one or more markers of memory cells, is desired, the method includes stopping the administration if memory cells, e.g., one or more markers of memory cells, are not increased at least 5%, 10%, 15%, 20%, 30%, 40%, 50% or more; or the method includes increasing the periodicity of administration if the memory cells, e.g., one or more markers of memory cells, are not increased at least 5%, 10%, 15%, 20% or more; or the method includes increasing the dose of one or both of the agent that modulates T cell function and the inhibitor of immune checkpoint if memory cells, e.g., one or more markers of memory cells, are not increased at least 5%, 10%, 15%, 20% or more.

In one embodiment the agent that modulates T cell function and the inhibitor of immune checkpoint are administered at a dosage and frequency sufficient to increase memory cells, e.g., one or more markers of memory cells, in the subject. In certain embodiments, memory cells, e.g., one or more markers of memory cells, are increased at least 1%, 2%, 5%, 10%, 15%, 20%, 25%, 30%, 35%, 40%, 50%, 60%, 70%, 80% or more, compared to before the administration. In certain embodiments, memory cells, e.g., one or more markers of memory cells, are increased between 5-20%, between 5-50%, between 10-50%, between 20-80%, between 20-70%.

In another aspect, the invention features a method of treating chronic infection in a subject. The method includes administering to the subject (i) an agent that modulates thymus or T-cell function in combination with (ii) an inhibitor of immune checkpoint.

In one embodiment, the method includes selecting or diagnosing a subject having a chronic infection, e.g., infection caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has a polyoma virus JC infection and/or has progressive multifocal leukoencephalopathy (PML). In some embodiments, the subject has been treated with an anti-alpha(4)-integrin agent, such as an anti- alpha(4)-integrin monoclonal antibody (e.g., natalizumab).

In some embodiments, the subject is also treated with interferon or antiviral drugs. In on embodiment, the subject has also been diagnosed with cancer.

In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BM P4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule. In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In other embodiments, the subject has not been diagnosed with cancer. In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the method also includes assessing the subject prior to treatment or first administration and using the results of the assessment to select a subject for treatment. The subject may be assessed for one or more of the following: (a) one or more markers of T cell exhaustion; (b) tumor infiltrating lymphocytes (TILs); (c) one or more markers of T cell diversity; (d) one or more markers of T cell clonality; (e) one or more markers of thymocytes; (f) thymus size; (g) one or more markers of thymic epithelial cells; (h) one or more markers of thymus stromal cells; (i) CAR-T cell persistence and/or function; (j) one or more markers of memory T cells.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where increasing tumor infiltrating lymphocytes (TILs); one or more markers of T cell diversity; one or more markers of T cell clonality; one or more markers of thymocytes; thymus size; one or more markers of thymic epithelial cells; one or more markers of thymus stromal cells; CAR-T cell persistence and/or function; or one or more markers of memory T cells is desired, the method includes stopping the administration if one or more of these is not increased at least 5%, 10%, 15%, 20%, 30%, 40%, 50% or more; or the method includes increasing the periodicity of administration if one or more of these is not increased at least 5%, 10%, 15%, 20% or more; or the method includes increasing the dose of one or both of the agent that modulates T cell function and the inhibitor of immune checkpoint if one or more of these is not increased at least 5%, 10%, 15%, 20% or more. As another example, in embodiments where decreasing one or more markers of T cell exhaustion is desired, the method includes stopping the administration if one or more of these is not decreased at least 5%, 10%, 15%, 20%, 30%, 40%, 50% or more; or the method includes increasing the periodicity of administration if one or more markers of T cell exhaustion is not decreased at least 5%, 10%, 15%, 20% or more; or the method includes increasing the dose of one or both of the agent that modulates T cell function and the inhibitor of immune checkpoint if one or more marker of T cell exhaustion is not decreased at least 5%, 10%, 15%, 20% or more.

In one embodiment the agent that modulates T cell function and the inhibitor of immune checkpoint are administered at a dosage and frequency sufficient to decrease one or more markers of T cell exhaustion or increase one or more of tumor infiltrating lymphocytes (TILs); one or more markers of T cell diversity; one or more marker of T cell clonality; one or more markers of thymocytes; thymus size; one or more markers of thymic epithelial cells; one or more markers of thymus stromal cells; CAR-T cell persistence and/or function; or one or more markers of memory T cell the subject. In certain embodiments, these are increased or decreased as desired at least 1%, 2%, 5%, 10%, 15%, 20%, 25%, 30%, 35%, 40%, 50%, 60%, 70%, 80% or more, compared to before the administration. In certain embodiments, these are increased or decreased as desired between 5-20%, between 5-50%, between 10-50%, between 20-80%, between 20-70%.

In another aspect, the invention features methods of decreasing or reversing age-associated or induced thymic involution. The method includes administering to the subject (i) an agent that modulates thymus or T-cell function in combination with (ii) an inhibitor of immune checkpoint.

In one embodiment, the subject is a human adult at least 30, 35, 40, 45, 50, 60, 70, or 80 years old. In one embodiment, the method includes selecting a subject over 65 years old.

In one embodiment, the subject's thymus is assessed, e.g., assessing includes performing an MRI or CT scan on the thymus of the subject.

In one embodiment, the subject has an acute or chronic infection.

In one embodiment, the method includes selecting or diagnosing a subject having a chronic infection.

In one embodiment, the subject has also been diagnosed with cancer.

In one embodiment, the infection is caused by: human immunodeficiency virus (HIV), hepatitis C virus (HCV), hepatitis B virus (HBV), adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyoma virus BK, polyoma virus JC, measles virus, rubella virus, human T cell leukemia virus I, human T cell leukemia virus II, Leishmania, Toxoplasma, Trypanosoma, Plasmodium, Schistosoma, or Encephalitozoon. In some embodiments, the chronic infection is not a latent infection.

In one embodiment, the subject has a polyoma virus JC infection and/or has progressive multifocal leukoencephalopathy (PML). In some embodiments, the subject has been treated with an anti-alpha(4)-integrin agent, such as an anti- alpha(4)-integrin monoclonal antibody (e.g., natalizumab).

In some embodiments, the agent that modulates T cell function is selected from: an interleukin or functional fragment or derivative or agonist thereof (e.g., IL-7 or a functional fragment or derivative thereof (e.g., CYT99 or CYT107); IL-12 or a functional fragment or derivative thereof (e.g., DNA-based IL-12, e.g., Immunopulse™), IL-15 or a functional fragment or derivative thereof, IL-22 or a functional fragment or derivative thereof, IL-23 or a functional fragment or derivative thereof); an interleukin receptor or agonist thereof (e.g., CD127 or a functional fragment or derivative thereof, soluble IL-7Rα or a functional fragment or derivative thereof, a CD127 activating monoclonal antibody); a growth factor (e.g., keratinocyte growth factor (KGF) or a functional fragment or derivative thereof, Flt3L or a functional fragment or derivative thereof (e.g., palifermin), IGF-1 or a functional fragment or derivative thereof); a peptide hormone or a functional fragment thereof (e.g., Ghrelin/GH or a functional fragment or derivative thereof), a bone morphogenic protein or a functional fragment or derivative thereof (e.g., BM P4 or a functional fragment thereof), immunostimulatory amino acids, (e.g., arginine or an analog or derivative thereof), an agonist or antagonist of a releasing hormone or a releasing hormone receptor (e.g., aGnRH antagonist such as degarelix acetate or a GnRH agonist such as leuprolide), hormonal modifiers, (e.g., a hormone), an antiandrogen drug or chemical castration agent (e.g., cyproterone acetate), an aromatase inhibitor (e.g., a steroidal inhibitor such as exemestane or a non-steroidal inhibitor such as anastrozole or letrozole) an estrogen receptor agonist or antagonist (e.g., tamoxifen, toremifene, raloxifene, ormeloxifene, clomifene, lasofoxifene, ospemifene, or fulvestrant). Also included are any combinations of any of the above.

In some embodiments, the agent that modulates T cell function is a therapeutic mRNA, e.g., a therapeutic RNA that encodes IL-7 or a functional fragment or derivative thereof, CD127 or a functional fragment or derivative thereof, soluble IL-7Rα or a functional fragment or derivative thereof, a CD127 activating monoclonal antibody, an anti-IL-7 antibody, IL-12 or a functional fragment or derivative thereof, IL-22 or a functional fragment or derivative thereof, IL-23 or a functional fragment or derivative thereof, KGF or a functional fragment or derivative thereof, Flt3L or a functional fragment or derivative thereof, IGF-1 or a functional fragment or derivative thereof, Ghrelin/GH or a functional fragment or derivative thereof, BMP-4 or a functional fragment or derivative thereof, IL-15 or a functional fragment or derivative thereof, arginine or an analog or derivative thereof, a hormone, a GnRH antagonist, a GnRH agonist, sex steroid ablation, an aromatase inhibitor, an estrogen receptor agonist or antagonist, and combinations thereof. Therapeutic mRNAs are described, e.g., in WO 2013/151666.

In some embodiments, the agent that modulates T cell function is an antibody or antigen-binding fragment thereof.

In some embodiments, the agent that modulates T cell function is a therapeutic small molecule. In one embodiment, the inhibitor of checkpoint is an inhibitory antibody (e.g., a monospecific antibody such as a monoclonal antibody). The antibody may be, e.g., humanized or fully human.

In other embodiments, the inhibitor of checkpoint is a fusion protein, e.g., an Fc-receptor fusion protein.

In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

In one embodiment, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab).

In on embodiment, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011).

In one embodiment, the inhibitor of checkpoint is an inhibitor (e.g., an inhibitory antibody or small molecule inhibitor) of PD-L1 (e.g., MPDL3280A/RG7446; MED14736; MSB0010718C; BMS 936559).

In one embodiment, the inhibitor of checkpoint is an inhibitor (e.g., an inhibitory antibody or Fc fusion or small molecule inhibitor) of PD-L2 (e.g., a PD-L2/Ig fusion protein such as AMP 224).

In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In one embodiment, the agent is IL-7 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL-22 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is CD127 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is soluble IL-7R or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL12 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL15 or a functional fragment or derivative thereof and the checkpoint inhibitor is an anti PD-1 antibody, and anti-PD-L1 antibody, an anti-PD-L2 antibody or an anti-CTLA4 antibody. In one embodiment, the agent is IL23 or a functional fragment or derivative thereof and the checkpoint inhibitor is an anti PD-1 antibody, and anti-PD-L1 antibody, an anti-PD-L2 antibody or an anti-CTLA4 antibody.

In some embodiments, the method further includes assessing one or more (e.g., 2, 3, 4, 5, 6 or more) transcription factors selected from: eomesodermin, T-bet, GATA-3, BCL-6, Helios, NFAT, Blimp-1, FoxO1, c-myc, or a combination thereof.

In one embodiment, the subject has been diagnosed with cancer, e.g., a hematological cancer or a solid cancer such as lung cancer, non-small cell lung cancer (NSCLC), skin cancer, melanoma, cervical cancer, uterine cancer, ovarian cancer, breast cancer, pancreatic cancer, stomach cancer, esophageal cancer, colorectal cancer, liver cancer, prostate cancer, kidney cancer, bladder cancer, head and neck cancer, sarcoma, lymphoma, and brain cancer. In certain embodiments, the method further comprises performing surgery (e.g., to resect the cancer tissue), radiation therapy, cryotherapy or hyperthermia therapy on the subject. In some embodiments, the subject is additionally assessed for a clinical outcome such as tumor growth, tumor regression; tumor shrinkage; tumor necrosis; tumor metastasis.

In one embodiment, the subject has thymic involution associated with a therapy, e.g., a cancer therapy. For example, the subject is receiving or has received chemotherapy, radiation therapy, hormone therapy, cryotherapy, hyperthermia therapy or any other cancer therapy described herein.

In other embodiments, the subject has not been diagnosed with cancer.

In some embodiments, the subject is a juvenile, e.g., a human subject less than 18 years old, e.g., less than 16, 15, 14, 13, 12, 11, 10, 9, 8, 7, 6, 5, 4, 3, 2 years old. In other embodiments the subject is an adult, e.g., an adult older than 18, 25, 35, 40, 50, 60, 70, 80, or 85.

In one embodiment, the method also includes assessing the subject prior to treatment or first administration and using the results of the assessment to select a subject for treatment. The subject may be assessed for one or more of the following: (a) one or more markers of T cell exhaustion; (b) tumor infiltrating lymphocytes (TILs); (c) one or more markers of T cell diversity; (d) one or more markers of T cell clonality; (e) one or more markers of thymocytes; (f) thymus size; (g) one or more markers of thymic epithelial cells; (h) one or more markers of thymus stromal cells; (i) CAR-T cell persistence and/or function; (j) one or more markers of memory T cells.

In one embodiment, the method also includes modifying the administering step (e.g., stopping the administration, increasing or decreasing the periodicity of administration, increasing or decreasing the dose of one or both of the agent that modulates T cell function and the inhibitor of immune checkpoint) based on the results of the assessment. For example, in embodiments where increasing tumor infiltrating lymphocytes (TILs); one or more markers of T cell diversity; one or more markers of T cell clonality; one or more markers of thymocytes; thymus size; one or more markers of thymic epithelial cells; one or more markers of thymus stromal cells; CAR-T cell persistence and/or function; or one or more markers of memory T cells is desired, the method includes stopping the administration if one or more of these is not increased at least 5%, 10%, 15%, 20%, 30%, 40%, 50% or more; or the method includes increasing the periodicity of administration if one or more of these is not increased at least 5%, 10%, 15%, 20% or more; or the method includes increasing the dose of one or both of the agent that modulates T cell function and the inhibitor of immune checkpoint if one or more of these is not increased at least 5%, 10%, 15%, 20% or more. As another example, in embodiments where decreasing one or more markers of T cell exhaustion is desired, the method includes stopping the administration if one or more of these is not decreased at least 5%, 10%, 15%, 20%, 30%, 40%, 50% or more; or the method includes increasing the periodicity of administration if one or more markers of T cell exhaustion is not decreased at least 5%, 10%, 15%, 20% or more; or the method includes increasing the dose of one or both of the agent that modulates T cell function and the inhibitor of immune checkpoint if one or more markers of T cell exhaustion is not decreased at least 5%, 10%, 15%, 20% or more.

In one embodiment the agent that modulates T cell function and the inhibitor of immune checkpoint are administered at a dosage and frequency sufficient to decrease one or more markers of T cell exhaustion or increase one or more of tumor infiltrating lymphocytes (TILs); one or more markers of T cell diversity; one or more markers of T cell clonality; one or more markers of thymocytes; thymus size; one or more markers of thymic epithelial cells; one or more markers of thymus stromal cells; CAR-T cell persistence and/or function; or one or more markers of memory T cell the subject. In certain embodiments, these are increased or decreased as desired at least 1%, 2%, 5%, 10%, 15%, 20%, 25%, 30%, 35%, 40%, 50%, 60%, 70%, 80% or more, compared to before the administration. In certain embodiments, these are increased or decreased as desired between 5-20%, between 5-50%, between 10-50%, between 20-80%, between 20-70%.

In another aspect, the invention features a method of treating a subject having a low, moderate or highly immunogenic cancer. The method includes administering to the subject an agent that modulates thymus or T-cell function in combination with an inhibitor of checkpoint. In some embodiments, the method includes assessing (or having assessed) the subject's cancer for level of immunogenicity.

In some embodiments, the subject has a low immunogenicity cancer. In other embodiments, the subject has a moderate immunogenicity cancer. In yet other embodiments, the subject has a high immunogenicity cancer (e.g., a metastatic or stage III or stage IV cancer that originated in the lung, colon, skin, liver, or brain).

In some embodiments, the level of immunogenicity of the cancer is assessed by DNA sequencing of biopsied cancer tissue and comparing the DNA sequence obtained to that of somatic DNA from the same subject. In other embodiments, the level of immunogenicity of the cancer is assessed by mRNA sequencing of the cancer tissue and analyzing the results to determine the abundance of immune signatures associated with immunogenicity. In other embodiments, the level of immunogenicity of the cancer is assessed by Immunoscore™. In other embodiments, the level of immunogenicity of the cancer is assessed by tumor-infiltrating lymphocyte (TIL) grading.

In another aspect, the invention features a reaction mixture that includes (a) a biological sample from a subject who is being treated or has been treated with an agent that modulates thymus or T-cell function in combination with an inhibitor of immune checkpoint and (b) a probe directed to one or more of: a marker of T cell exhaustion (e.g., a marker of T cell exhaustion described herein); a tumor infiltrating lymphocyte; a marker of T cell diversity (e.g., a marker of T cell diversity described herein); a marker of T cell clonality (e.g., a marker of T cell clonality described herein); a marker of thymocytes (e.g., a marker of thymocytes described herein); a marker of thymic epithelial cells (e.g., a marker of thymic epithelial cells described herein); a marker of thymus stromal cells (e.g., a marker of thymic stromal cells described herein); a marker of CAR-T persistence and/or function (e.g., a marker of CAR-T persistence described herein); a marker of memory T cells (e.g., a marker of memory T cells described herein).

In some embodiments, the sample is from a cancer patient. In some embodiments, the sample is from a cancer patient in remission. In some embodiments, the sample is from a patient who has been diagnosed with a chronic infection.

In some embodiments, the probe is an antibody that detects a marker of T cell exhaustion; a tumor infiltrating lymphocyte; a marker of T cell diversity; a marker of T cell clonality; a marker of thymocytes; a marker of thymic epithelial cells; a marker of thymus stromal cells; a marker of CAR-T persistence and/or function; a marker of memory T cells.

In other embodiments, the probe is a nuclei acid that detects (e.g., hybridizes to and/or amplifies) a nucleic acid encoding a marker of T cell exhaustion; a tumor infiltrating lymphocyte; a marker of T cell diversity; a marker of T cell clonality; a marker of thymocytes; a marker of thymic epithelial cells; a marker of thymus stromal cells; a marker of CAR-T persistence and/or function; a marker of memory T cells.

In another aspect, the invention features a method of selecting a subject for treatment with an agent that modulates thymus or T-cell function (e.g., an agent that modulates thymus or T-cell function described herein) in combination with an inhibitor of immune checkpoint (e.g., an inhibitor of checkpoint described herein). The method includes

(a) identifying a subject who has a tumor or cancer;

(b) assessing (or having assessed) the subject for inadequate baseline immune function, e.g., assessing (or having assessed) a biological sample from the subject for one or more of: a marker of T cell exhaustion (e.g., a marker of T cell exhaustion described herein); a marker of T cell cytokine receptor expression (e.g., a marker of T cell cytokine expression as described herein); a marker of tumor cytokine expression (e.g., a marker of tumor cytokine expression as described herein); a marker of tumor infiltrating lymphocytes (e.g., a marker of tumor infiltrating lymphocytes described herein); a marker of T cell diversity (e.g., a marker of T cell diversity described herein); a marker of T cell clonality (e.g., a marker of T cell clonality described herein); a marker of thymocytes (e.g., a marker of thymocytes described herein); a marker of thymic epithelial cells (e.g., a marker of thymic epithelial cells described herein); a marker of thymus stromal cells (e.g., a marker of thymic stromal cells described herein); a marker of CAR-T persistence and/or function (e.g., a marker of CAR-T persistence described herein); a marker of memory T cells (e.g., a marker of memory T cells described herein);

(c) selecting the subject for treatment with the agent that modulates thymus or T-cell function in combination with the inhibitor of immune checkpoint, e.g., prescribing to the subject treatment with the agent that modulates thymus or T-cell function in combination with the inhibitor of immune checkpoint based on the assessment; and

(d) optionally further treating the subject with the agent that modulates thymus or T-cell function in combination with the inhibitor of immune checkpoint.

In some embodiments, the biological sample assessed is a biopsied tumor. In some embodiments, the biological sample assessed is a biopsied tumor-draining lymph node. In some embodiments, the biological sample assessed is the subject's peripheral blood. In one embodiment, the peripheral blood is withdrawn before assessment.

In one embodiment the subject is selected if the subject has TILs comprising T cells and/or NK cells expressing high CD127 (e.g., 10%, 25%, 50%, 75%, 100% higher as compared to a non-memory effector T cell or NK cell population from a normal control).

In some embodiments, the subject is selected if the assessed tissue or blood has T cells with high expression (e.g., 10%, 25%, 50%, 75%, 100% higher as compared to T cells from healthy tissue) of one or more (e.g., 2, 3, 4, 5, 6 or more) cytokine receptor chains that are selected from: CD127, CD25, CD122, CD124, CD122, CD360, CD132, and a combination thereof. In some embodiments, the subject is selected if the assessed tissue or blood has T cells with high expression (e.g., 10%, 25%, 50%, 75%, 100% higher as compared to T cells from healthy tissue) of the common gamma chain receptor (CD132).

In some embodiments, the subject is selected if the subject's biopsied tumor has low expression (e.g., 10%, 25%, 50%, 75%, 100% lower as compared to equivalent healthy tissue, or lower than a predetermined threshold value) of one or more (e.g., 2, 3, 4, 5, 6 or more) cytokines that are selected from: IL-7, IL-2, IL-4, IL-9, IL-15, IL-21, and a combination thereof. In one embodiment, the subject is selected if the subject's biopsied tumor has low expression (e.g., 10%, 25%, 50%, 75%, 100% lower as compared to equivalent healthy tissue) of IL-7, or lower than a predetermined threshold value. In one embodiment, the subject is selected if the subject's biopsied tumor has low expression (e.g., 10%, 25%, 50%, 75%, 100% lower as compared to equivalent healthy tissue, or lower than a predetermined threshold value) of IL-7 receptor (e.g., CD127).

In some embodiments, the subject is selected if the subject's biopsied tumor has a low ratio of expression of one or more (e.g., 2, 3, 4, 5, 6 or more) cytokines relative to expression of a component of the cytokine's cognate receptor on TILs present in the biopsied tumor (e.g., 10%, 25%, 50%, 75%, 100% lower relative expression of the cytokine and its cognate receptor component compared the relative expression of the same cytokine and cytokine receptor component in equivalent healthy tissue). The ratio of expression is measured using one or more (e.g., 2, 3, 4, 5, 6) pairs of cytokines and receptor components selected from: IL-7/CD127, IL-2/CD25, IL-2/CD122, IL-4/CD124, IL-15/CD122, and IL-21/CD360. In one embodiment, the subject is selected if the subject's biopsied tumor has a low ratio of expression of IL-7 relative to expression of IL-7R (CD127) on TILs present in the biopsied tumor.

In one embodiment, the subject is selected if the subject has TILs exhibiting low T cell diversity (e.g., 10%, 25%, 50%, 75%, 100% lower, as compared to control populations of TILs from checkpoint inhibitor responders). (PMID 25428505)

In one embodiment, the subject is selected if the subject has TILs exhibiting low T cell clonality (e.g., 10%, 25%, 50%, 75%, 100% lower respectively, as compared to control populations of TILs from checkpoint inhibitor responders). (PMID 25428505)

In one embodiment, the subject is selected if the subject has TILs exhibiting high T cell diversity and low T cell clonality, (e.g., 10%, 25%, 50%, 75%, 100% higher or lower, respectively, as compared to control populations of TILs from checkpoint inhibitor responders). (PMID 25428505)

In one embodiment, the method also includes assessing the cancer for immunogenicity and selecting the subject for the treatment if the subject has a highly immunogenic tumor or cancer.

In other embodiments, the method also includes staging the cancer and selecting the subject for the treatment if the subject has metastatic or stage III or stage IV cancer, e.g., metastatic or stage III or stage IV cancer that originated in the lung, colon, skin, liver, or brain.

In other embodiments, the method includes staging the cancer and selecting the subject for the treatment if the subject has metastatic or stage III or stage IV cancer that originated in the lung, colon, skin, liver, or brain.

In another aspect, the invention features a method of treating cancer in a subject comprising administering to the subject an agent that modulates thymus or T-cell function (e.g., an agent that modulates thymus or T-cell function described herein) in combination with an inhibitor of immune checkpoint (e.g., an inhibitor of checkpoint described herein). The method includes:

(a) identifying a subject who has a tumor or cancer having one or more (2, 3 4 or more of):
1. a predetermined threshold value of TILs expressing high CD127 (e.g., 10%, 25%, 50%, 75%, 100% higher as compared to a non-memory effector cell population from a normal control);
2. TILs expressing a predetermined threshold value of CD127;
3. T cells with predetermined threshold value of CD127, CD25, CD122, CD124, CD122, CD360, CD132, or a combination thereof, e.g., high expression (e.g., 10%, 25%, 50%, 75%, 100% higher as compared to T cells from healthy tissue) of one or more (e.g., 2, 3, 4, 5, 6 or more) cytokine receptor chains that are selected from: CD127, CD25, CD122, CD124, CD122, CD360, CD132, or combinations thereof;
4. low expression (e.g., 10%, 25%, 50%, 75%, 100% lower as compared to equivalent healthy tissue, or lower than a predetermined threshold value) of one or more (e.g., 2, 3, 4, 5, 6 or more) cytokines that are selected from: IL-7, IL-2, IL-4, IL-9, IL-15, IL-21, and a combination thereof;
5. low expression (e.g., 10%, 25%, 50%, 75%, 100% lower as compared to equivalent healthy tissue) of IL-7;
6. low expression (e.g., 10%, 25%, 50%, 75%, 100% lower as compared to equivalent healthy tissue, or lower than a predetermined threshold value) of IL-7 receptor (e.g., CD127);
7. a low ratio of expression of one or more (e.g., 2, 3, 4, 5, 6 or more) cytokines relative to expression of a component of the cytokine's cognate receptor on TILs present in the biopsied tumor (e.g., 10%, 25%, 50%, 75%, 100% lower relative expression of the cytokine and its cognate receptor component compared the relative expression of the same cytokine and cytokine receptor component in equivalent healthy tissue). (The ratio of expression is measured using one or more (e.g., 2, 3, 4, 5, 6) pairs of cytokines and receptor components selected from: IL-7/CD127, IL-2/CD25, IL-2/CD122, IL-4/CD124, IL-15/CD122, and IL-21/CD360); and

(b) treating the subject with the agent that modulates thymus or T-cell function (e.g., IL-7) in combination with the inhibitor of immune checkpoint.

In some embodiments, the subject is assessed for one or more of (a) 1-7 before, during or after the administration, e.g., the subject's biological sample is assessed (e.g., a biopsied tumor, a tumor-draining lymph node, peripheral blood).

In another aspect, the invention features a method of treating a subject in need thereof with a combination of a checkpoint inhibitor (e.g., a checkpoint inhibitor described herein) and an agent that modulates T cell function (e.g., an agent that modulates T cell function described herein), wherein the combination results in synergistic improvement of one or more assessments of T cell function described herein, in the subject.

In some embodiments, the assessment is a marker of T cell exhaustion (e.g., a marker of T cell exhaustion described herein); tumor infiltrating lymphocytes; a marker of T cell diversity (e.g., a marker of T cell diversity described herein or a metric for T cell receptor repertoire diversity); a marker of T cell clonality (e.g., a marker of T cell clonality described herein); a marker of thymocytes (e.g., a marker of thymocytes described herein); a marker of thymic epithelial cells (e.g., a marker of thymic epithelial cells described herein); a marker of thymus stromal cells (e.g., a marker of thymic stromal cells described herein); a marker of CAR-T persistence and/or function (e.g., a marker of CAR-T persistence described herein); a marker of memory T cells (e.g., a marker of memory T cells described herein).

In one embodiment, the assessment is performed in a bone marrow chimeric lab animal with human T cells (e.g., a humanized bone marrow model (mouse reconstituted with human cells), see e.g., http://clincancerres.aacrjournals.org/content/17/8/2195.full). In one embodiment, the assessment is performed in an in vitro or cell-based assay of T cell function described herein. In one embodiment, the subject has cancer and/or a chronic infection (e.g., a cancer or chronic infection described herein).

In another aspect, the invention features a composition (e.g., a pharmaceutical composition) comprising a checkpoint inhibitor (e.g., a checkpoint inhibitor described herein) and an agent that modulates T cell function (e.g., an agent that modulates T cell function described herein), wherein the composition results in synergistic improvement of one or more assessments of T cell function described herein (e.g., in an in vitro or cell based assay of T cell function described herein; or in vivo in a subject).

In another aspect, the invention features a method of treatment comprising administering a checkpoint inhibitor (e.g., a checkpoint inhibitor described herein) in combination with an agent that modulates T cell function (e.g., an agent that modulates T cell function described herein) to a subject, and assessing the subject for one or more adverse symptoms or conditions described herein.

In some embodiments, the subject is assessed for presence of, or a symptom of: auto-immune disease, e.g., multiple sclerosis, rheumatoid arthritis, systemic lupus erythematosus, scleroderma, psoriasis, celiac disease, vitiligo, Hashimoto's disease (autoimmune thyroiditis), Addison's disease, Grave's disease, Sjogren's syndrome, or type 1 diabetes.

In some embodiments, the subject has a pre-existing auto-immune disease, e.g., multiple sclerosis, rheumatoid arthritis, systemic lupus erythematosus, scleroderma, psoriasis, celiac disease, vitiligo, Hashimoto's disease (autoimmune thyroiditis), Addison's disease, Grave's disease, Sjogren's syndrome, or type 1 diabetes, and the assessing comprises assessing the subject for an increase in one or more disease-associated symptoms (e.g., increased inflammation in the synovial membrane of affected joints in subjects with pre-existing rheumatoid arthritis; increased relapse rate, increased optic neuritis, reduced ability to keep balance and walk in subjects with pre-existing multiple sclerosis; increased skin redness, increased skin irritation, increased surface area of thick, flaky, and/or silver-white patches of skin in subjects with pre-existing psoriasis; increased abdominal bloating and pain, increased frequency and/or severity of diarrhea, increased fatigue, increased dermatitis herpetiformis in subjects with pre-existing celiac disease).

In some embodiments, the subject is assessed for one or more immune-related symptoms, e.g., immune-mediated endocrinopathy, immune-mediated pneumonitis, immune-mediated colitis, immune-mediated hepatitis, immune-mediated nephritis and renal dysfunction, immune-mediated skin adverse reactions, encephalitis, or complications of an allogeneic hematopoetic stem cell therapy.

### Definitions:

As used herein, an “antibody” is a protein that includes at least one immunoglobulin variable domain or immunoglobulin variable domain sequence. For example, an antibody can include a heavy (H) chain variable region (abbreviated herein as VH), and a light (L) chain variable region: (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies. An antibody can have the structural features of IgA, IgG, IgE, IgD, IgM (as well as subtypes thereof). In some embodiments, antibodies are recombinant human or humanized antibodies.

The term “chimeric antigen receptor-T cell” or alternatively a “CAR-T” refers to an immune effector cell, a T cell, with specificity for a target cell, typically a cancer cell, provided by a chimeric antigen receptor or CAR on the i cell. The CAR provides a primary signal to the T cell by binding the stimulatory domain of the CAR with its ligand (e.g., tumor antigen in the case of a CAR) thereby mediating a signal transduction event, e.g., signaling through the intracellular domains of the CAR to activate cytotoxic function, in the T cell.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the phrase “conservative amino acid substitution” refers to an amino acid residue replaced with an amino acid, amino acid analog, or modified amino acid having a similar side chain. Families of amino acids having similar side chains are defined in the art. For example, families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). A predicted nonessential amino acid in a polypeptide, for example, is replaced with another amino acid, amino acid analog, or modified amino acid having a similar side chain. Other examples of acceptable substitutions are substitutions based on isosteric considerations (e.g. norleucine for methionine) or other properties (e.g. 2-thienylalanine for phenylalanine).

As used herein, the term “derivative” or “variant” of a polypeptide refers to a polypeptide having at least one sequence difference compared to that polypeptide, e.g., one or more substitutions, insertions, or deletions. In some embodiments, the derivative has at least 70%, 80%, 85%, 90%, 95%, 96%, 97%, 98%, or 99% identity to that polypeptide. A derivative includes a fragment

As used herein, the term “fragment” refers to a portion of a parent polypeptide, e.g., one or more domains. In some embodiments, the fragment has at least 70%, 80%, 85%, 90%, 95%, 96%, 97%, 98%, or 99% of the parent polypeptide. In some embodiments, a fragment lacks up to 1, 2, 3, 4, 5, 10, 20, or 100 amino acids on the N-terminus, C-terminus, or both (each independently), compared to the full-length polypeptide. A “functional fragment” refers to the portion that maintains at least one function of the parent polypeptide, e.g., biologically active portion. In some embodiments, the functional fragment has at least 70%, 80%, 85%, 90%, 95%, 96%, 97%, 98%, or 99% of the function of the parent polypeptide.

The term “immunogenic cancer” refers to the relative abundance of a tumor expressing key immune genes, such as CD3, CD8a, GZMB, IFNG and others. High relative cumulative abundance of these transcripts provides for the characterization of the tumor as highly immunogenic, whereas low abundance or absence of these transcripts provides for characterization of the tumor as poorly immunogenic.

As used herein, the terms “increasing” and “decreasing” refer to modulating resulting in, respectively, greater or lesser amounts, function or activity of a metric relative to a reference. For example, subsequent to administration of an agent that modulates T cell function in combination with an inhibitor of immune checkpoint, the amount of a marker of T cell exhaustion may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, 6 months, after a treatment regimen (e.g., a combination therapy described herein) has begun.

As used herein, the phrases “inhibitor of immune checkpoint” and “checkpoint inhibitor” are used interchangeably herein and refer to an agent that blocks a suppressive or inhibitory pathway in immune cells, activates a stimulatory pathway in immune cells, depletes suppressive populations of immune cells, or blocks a suppressive immune pathway on cancer cells.

As used herein, the term “pharmaceutical composition” refers to a medicinal or pharmaceutical formulation that contains one or more active ingredient as well as one or more excipients and diluents to enable the active ingredient(s) suitable for the method of administration. The pharmaceutical composition of the present invention includes pharmaceutically acceptable components that are compatible with the agents described herein. The pharmaceutical composition is typically in aqueous form for intravenous or subcutaneous administration. A pharmaceutical composition or pharmaceutical preparation produced under good manufacturing practices (GMP) conditions, having pharmacological activity or other direct effect in the mitigation, treatment, or prevention of disease, and/or a finished dosage form or formulation thereof and is for human use.

The term “synergy” or “synergistic” means a more than additive effect of a combination of two or more agents (e.g., a combination therapy described herein) compared to their individual effects. In certain embodiments, synergistic activity is present when a first agent produces a detectable level of an output X, a second agent produces a detectable level of the output X, and the first and second agents together produce a more-than-additive level of the output X.

As used herein, the phrase “TCR repertoire diversity” refers to the number of different T cell receptors (TCR) in a population of T cells; sources of diversity within TCR repertoire include unique alpha and beta TCR subunits, genetic differences stemming from V-J (TCR alpha) and V-D-J (TCR beta) recombination, as well as different terminal deoxynucleotidyl transferase (TdT)—introduced nucleotides at DNA junctions, resulting in sequence variation.

As used herein, the phrase “T cell clonality” refers to the absolute or relative size of a clonal T cell population or of several clonal T cell populations, including peripheral blood mononuclear cells, TILs, or other tissue-derived immune cells, or the magnitude of a T cell expansion giving rise to said clonal population.

As used herein, the phrase “T cell exhaustion” refers to T cell dysfunction or a dysfunctional state of T cells indicated by reduced or absent production of effector cytokines IFNg, TNFa, IL-2, effector molecules perforin, granzyme A, B, or K, reduced ability of cells to proliferate, and increased expression of co-inhibitory receptors PD-1, LAG-3, TIM-3, CTLA-4, CD160, 2B4 (CD244), BTLA, or TIGIT.

“Treatment” and “treating,” as used herein, refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy).

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 is a panel of images showing protein expression of IL-7, including an artificial signal peptide and a His tag, via SDS-PAGE on the left and western blot on the right.

FIG. 2 is a panel of images showing protein expression of IL-7-Fc-receptor fusion (IL-7-Fc), including an artificial signal peptide, via SDS-PAGE on the left and western blot on the right.

FIG. 3 shows mean tumor burden in mice treated with isotype control, 0.2 mg/injection, intraperitoneal (IP) (Group 1); anti-mPD-L1 Ab, 0.2 mg/injection, IP (Group 2); rhIL-7-His, 0.02 mg/injection, IP (Group 3); anti-mPD-L1 Ab+IL-7-His, 0.2 mg/injection+0.08mg/injection, IP+IP (Group 4); and anti-mPD-L1 Ab+IL-7-His, 0.2 mg/injection+0.02 mg/injection, IP+IP (Group 5).

FIG. 4 is an image showing the relative expression level of IL7 and IL-7R in different cancer types.

### DETAILED DESCRIPTION OF THE INVENTION

The invention relates to therapeutic methods, e.g., combination therapies, that can improve certain thymic parameters, functions and/or activities in a subject in need thereof. A therapeutic regimen that includes administering an agent that modulates thymus or T-cell function in combination with an inhibitor of immune checkpoint can be used to modulate (e.g., increase or decrease, as desired) T cell exhaustion, tumor infiltrating lymphocytes, T-cell diversity, T cell clonality, thymocytes, thymic epithelial cells, thymic stromal cells, and/or thymus size, thereby improving immune function in a subject, e.g., a patient such as a cancer patient. Such subjects may be monitored or assessed for such thymic parameters, functions and/or activities. In addition, combination therapy methods described herein may be used to improve B cell function, to treat chronic infection, and to improve immune function generally in subjects wherein age related thymic involution has impaired general immune response.

### Agents That Modulate Thymic Function

Agents that modulate thymic function described herein are agents that can increase or decrease one or more activity related to T cell development or function. Such an agent useful in the invention improves thymic or T cell function in combination, e.g., in a synergistic manner, with a checkpoint inhibitor.

Such agents include certain interleukins (e.g., IL-7, IL-12, IL-15, IL-22, IL-23 or their functional fragments or derivatives). Such interleukins can be administered as agents or the agent may be a nucleic acid, such as a therapeutic RNA encoding the interleukin. Also included are agonists of such interleukins, e.g., a small molecule agonist of IL-7, IL-12, IL-22, IL-23 or their receptors (e.g., CD127).

In some embodiments, the agent comprises an amino acid sequence of human IL-7, a sequence comprising DCDIEGKDGKQYESVLMVSIDQLLDSMKEIGSNCLNNEFNFFKRHICDANKEGMFLFRAARKLRQFLKMNSTGDFDLH LLKVSEGTTILLNCTGQVKGRKPAALGEAQPTKSLEENKSLKEQKKLNDLCFLKRLLQEIKTCWNKILMGTKEH (SEQ ID NO: 1), or a sequence having at least 70%, 80%, 85%, 90%, 95%, 96%, 97%, 98%, or 99% identity to SEQ ID NO: 1.

In some embodiments, the agent comprises an IL-7 isoform. In one embodiment, the agent comprises an amino acid sequence of an IL-7 isoform, a sequence comprising DCDIEGKDGKQYESVLMVSIDQLLDSMKEIGSNCLNNEFNFFKRHICDANKVKGRKPAALGEAQPTKSLEENKSLKEQK KLNDLCFLKRLLQEIKTCWNKILMGTKEH (SEQ ID NO: 2), or a sequence having at least 70%, 80%, 85%, 90%, 95%, 96%, 97%, 98%, or 99% identity to SEQ ID NO: 2. In another embodiment, the agent comprises an amino acid sequence of an IL-7 isoform, a sequence comprising DCDIEGKDGKQYESVLMVSIDQLLDSMKEIGSNCLNNEFNFFKRHICDANKEGMFLFRAARKLRQFLKMNSTGDFDLH LLKVSEGTTILLNCTGQEENKSLKEQKKLNDLCFLKRLLQEIKTCWNKILMGTKEH (SEQ ID NO: 3), or a sequence having at least 70%, 80%, 85%, 90%, 95%, 96%, 97%, 98%, or 99% identity to SEQ ID NO: 3. In another embodiment, the agent comprises an amino acid sequence of an IL-7 isoform, a sequence comprising DCDIEGKDGKQYESVLMVSIDQLLDSMKEIGSNCLNNEFNFFKRHICDANKEENKSLKEQKKLNDLCFLKRLLQEIKTCW NKILMGTKEH (SEQ ID NO: 4), or a sequence having at least 70%, 80%, 85%, 90%, 95%, 96%, 97%, 98%, or 99% identity to SEQ ID NO: 4.

In some embodiments, the agent comprises a derivative of IL-7.

In one embodiment, the agent comprises a derivative of IL-7, a sequence comprising DCDIEGKDGKQYESVLMVSIDQLLDSMKEIGSNCLNNEFNFFKRHICDANKEGMFLFRAARKLRQFLKMNSTGDFDLH LLKVSEGTTILLKEQKKLNDLCFLKRLLQEIKTCWNKILMGTKEH (SEQ ID NO: 5), or a sequence having at least 70%, 80%, 85%, 90%, 95%, 96%, 97%, 98%, or 99% identity to SEQ ID NO: 5. In some embodiments, the agent comprises a derivative of IL-7, a sequence comprising DCDIEGKDGKQYESVLMVSIDQLLDSMKEIGSNCLNNEFNFFKRHICDANKEGMFLFRAARKLRQFLKMNSTGDFDLH LLKVSEGTTILLNCTGQEENKSLKEQKKLNDLCFLKRLLQEIKTCWNKILMGTKEH (SEQ ID NO: 6), or a sequence having at least 70%, 80%, 85%, 90%, 95%, 96%, 97%, 98%, or 99% identity to SEQ ID NO: 6. In another embodiment, the agent comprises a derivative of IL-7, a sequence comprising DCDIEGKDGKQYESVLMVSIDQLLDSMKEIGSNCLNNEFNFFKRHICDANKEGMFLFRAARKLRQFLKMNSTGDFDLH LLKVSEGTTILLNCTGQH (SEQ ID NO: 7), or a sequence having at least 70%, 80%, 85%, 90%, 95%, 96%, 97%, 98%, or 99% identity to SEQ ID NO: 7. In another embodiment, the agent comprises a derivative of IL-7, a sequence comprising DCDIEGKDGKQYESVLMVSIDQLLDSMKEIGSNCLNNEFNFFKRHICDANKVKGRKPAALGEAQPTKSLEENKSLKEQK KLNDLCFLKRLLQEIKTCWNKILMGTKEH (SEQ ID NO: 8), or a sequence having at least 70%, 80%, 85%, 90%, 95%, 96%, 97%, 98%, or 99% identity to SEQ ID NO: 8. In another embodiment, the agent comprises a derivative of IL-7, a sequence comprising DCDIEGKDGKQYESVLMVSIDQLLDSMKEIGSNCLNNEFNFFKRHISDANKVKGRKPAALGEAQPTKSLEENKSLKEQK KLNDLCFLKRLLQEIKTCWNKILMGTKEH (SEQ ID NO: 9), or a sequence having at least 70%, 80%, 85%, 90%, 95%, 96%, 97%, 98%, or 99% identity to SEQ ID NO: 9. In another embodiment, the agent comprises a derivative of IL-7, a sequence comprising M KEIGSNCLNNEFNFFKRHICDANKEGM FLFRAARKLRQFLKM NSTGDFDLHLLKVSEGTTILLNCTGQVKGRKPAALG EAQPTKSLEENKSLKEQKKLNDLCFLKRLLQEIKTCWNKILMGTKEH (SEQ ID NO: 10), or a sequence having at least 70%, 80%, 85%, 90%, 95%, 96%, 97%, 98%, or 99% identity to SEQ ID NO: 10.

Other agents include growth factors such as keratinocyte growth factor (KGF) or a functional fragment or derivative thereof (e.g., palifermin), BMP-4 or a functional fragment or derivative thereof, IGF-1 or a functional fragment or derivative thereof, Flt3L or a functional fragment or derivative thereof; amino acids such as arginine or an analog or derivative thereof; and hormonal modulators (Ghrelin/GH or a functional fragment or derivative thereof, a hormone, a GnRH antagonist, a GnRH agonist, an aromatase inhibitor, an estrogen receptor agonist or antagonist, and combinations thereof).

The agents also include hybrid or fusion proteins, for example, functional fusions of 2 or more interleukins described herein (e.g., an IL-7/IL-12 hybrid, e.g., a protein having an N terminus of IL-7 and a C-terminus of IL-12; or a fusion of a functional IL-7 with a functional IL-12).

In some embodiments, the agent comprises an IL-7 fusion. In one embodiment, the agent comprises an IL-7-HGF beta fusion, a sequence comprising DCDIEGKDGKQYESVLMVSIDQLLDSMKEIGSNCLNNEFNFFKRHICDANKEGM FLFRAARKLRQFLKMNSTGDFDLH LLKVSEGTTILLNCTGQVKGRKPAALGEAQPTKSLEENKSLKEQKKLNDLCFLKRLLQEIKTCWNKILMGTKEGGGGSGG GGSGGGGSVVNGIPTRTNIGWMVSLRYRNKHICGGSLIKESWVLTARQCFPSRDLKDYEAWLGIHDVHGRGDEKCKQ VLNVSQLVYGPEGSD LVLM KLARPAVLDD FVSTI DLPNYGCTI PEKTSCSVYGWGYTG LI NYDG LLRVAH LYI MGN EKCS QHHRGKVTLNESEICAGAEKIGSGPCEGDYGGPLVCEQHKMRMVLGVIVPGRGCAIPNRPGIFVRVAYYAKWIHKIILT YKVPQS (SEQ ID NO: 11), or a sequence having at least 70%, 80%, 85%, 90%, 95%, 96%, 97%, 98%, or 99% identity to SEQ ID NO: 11.

In another embodiment, the agent comprises an IL-7-IL-15 fusion, a sequence comprising DCDIEGKDGKQYESVLMVSIDQLLDSMKEIGSNCLNNEFNFFKRHICDANKEGMFLFRAARKLRQFLKMNSTGDFDLH LLKVSEGTTILLNCTGQVKGRKPAALGEAQPTKSLEENKSLKEQKKLNDLCFLKRLLQEIKTCWNKILMGTKEHGGGGSG GGGSGGGGSMNWVNVISDLKKIEDLIQSMHIDATLYTESDVHPSCKVTAMKCFLLELQVISLESGDASIHDTVENLIILA NNSLSSNGNVTESGCKECEELEEKNIKEFLQSFVHIVQMFINTS (SEQ ID NO: 12), or a sequence having at least 70%, 80%, 85%, 90%, 95%, 96%, 97%, 98%, or 99% identity to SEQ ID NO: 12.

In some embodiments, the agent is a fusion with a heterologous moiety (e.g., protein, non-protein) to stabilize the agent, increase half-hie, tag, etc. In one embodiment, the agent is a fusion with polyethylene glycol (PEG) to provide, for example, increased stability and/or efficacy of the agent. Methods for PEGylation known in the art can be used in the methods and compositions provided herein. Such methods include, but are not limited to, those provided in “Comparative Binding of Disulfde-Bridged PEG-Fabs” Khalili et al., Bioconjugate Chemistry (2012), 23(11), 2262-2277; “Site-Specific PEGylation at Histidine Tags” Cong et al., Bioconjugate Chemistry (2012), 23(2), 248-263; “Disulfide bridge based PEGylation of proteins” Brocchini et al., Advanced Drug Delivery Reviews (2008), 60(1), 3-12; “Site-Specific PEGylation of Protein Disulfide Bonds Using a Three-Carbon Bridge” Balan et al., Bioconjugate Chemistry (2007), 18(1), 61-76; “Site-specific PEGylation of native disulfide, bonds in therapeutic proteins” Shaunak et al., Nature Chemical Biology (2006), 2(6), 312-313; “PEG derivative conjugated proteins and peptides for use in pharmaceuticals” Godwin et al., WO 2010/100430, All of the aforementioned PEGylation references are incorporated by references in their entireties.

In one embodiment, the agent is a fusion with the Fc domain of IgG to provide, for example, increased half-life of the agent, e.g., plasma half-life. In another embodiment, the agent comprises an IL-7-Fc fusion, a sequence comprising DCDIEGKDGKQYESVLMVSIDQLLDSMKEIGSNCLNNEFNFFKRHICDANKEGMFLFRAARKLRQFLKMNSTGDFDLH LLKVSEGTTILLNCTGQVKGRKPAALGEAQPTKSLEENKSLKEQKKLNDLCFLKRLLQEIKTCWNKILMGTKEHGGGGSG GGGSGGGGSEPKSCDKTHTCPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQVYTLPPSRDELTKNQV SLTCLVKGFYPSDIAVEWESNGQPENNYKTTPPVLDSDGSFFLYSKLTVDKSRWQQGNVFSCSVMHEALHNHYTQKSL SLSPGK (SEQ ID NO: 13), or a sequence having at least 70%, 80%, 85%, 90%, 95%, 96%, 97%, 98%, or 99% identity to SEQ ID NO: 13.

In some embodiments, the agent comprises a precursor peptide. For example, an agent comprising IL-7 or an IL-7 isoform may include a precursor peptide comprising an amino acid sequence MFHVSFRYIFGLPPLILVLLPVASS (SEQ ID NO: 14), or a sequence having at least 70%, 80%, 85%, 90%, 95%, 96%, 97%, 98%, or 99% identity to SEQ ID NO: 14.

In some embodiments, the agent comprises a leader peptide. For example, an agent comprising IL-7 or an IL-7 isoform may include a leader peptide comprising an amino acid sequence MGWSCIILFLVATATGVHS (SEQ ID NO: 15), or a sequence having at least 70%, 80%, 85%, 90%, 95%, 96%, 97%, 98%, or 99% identity to SEQ ID NO: 15.

In some embodiments, the agent comprises a tag or marker. For example, an agent comprising IL-7 or an IL-7 isoform may include a tag or marker comprising an amino acid sequence HHHHHH (SEQ ID NO: 16), or a sequence having at least 70%, 80%, 85%, 90%, 95%, 96%, 97%, 98%, or 99% identity to SEQ ID NO: 16.

Such fusions may be engineered and expressed from one nucleic acid, and may be connected though a linker, such as a peptide linker, or covalently tethered through other means known to the art. The agents also include such hybrid molecules or fusions held together through non-covalent interactions. The linker may be a chemical bond, e.g., one or more covalent bonds or non-covalent bonds. In some embodiments, the linker is a peptide linker. Such a linker may be between 2-30 amino acids, or longer.

The linker includes flexible, rigid or cleavable linkers. The most commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of the linker in aqueous solutions by forming hydrogen bonds with the water molecules, and therefore reduce unfavorable interactions between the linker and the protein moieties.

Rigid linkers are useful to keep a fixed distance between domains and to maintain their independent functions. Rigid linkers may also be useful when a spatial separation of the domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as the presence of reducing reagents or proteases. In vivo cleavable linkers may utilize the reversible nature of a disulfide bond. One example includes a thrombin-sensitive sequence (e.g., PRS) between the two Cys residues. In vitro thrombin treatment of CPRSC results in the cleavage of the thrombin-sensitive sequence, while the reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under pathological conditions (e.g. cancer or inflammation), in specific cells or tissues, or constrained within certain cellular compartments. The specificity of many proteases offers slower cleavage of the linker in constrained compartments.

Examples of linking molecules include a hydrophobic linker, such as a negatively charged sulfonate group; lipids, such as a poly (—CH₂—) hydrocarbon chains, such as a PEG group, unsaturated variants thereof, hydroxylated variants thereof, amidated or otherwise N-containing variants thereof, noncarbon linkers; carbohydrate linkers; phosphodiester linkers, or other molecule capable of covalently linking two or more polypeptides. Non-covalent linkers are also included, such as hydrophobic lipid globules to which the polypeptide is linked, for example through a hydrophobic region of the polypeptide or a hydrophobic extension of the polypeptide, such as a series of residues rich in leucine, isoleucine, valine, or perhaps also alanine, phenylalanine, or even tyrosine, methionine, glycine or other hydrophobic residue. The polypeptide may be linked using charge-based chemistry, such that a positively charged moiety of the polypeptide is linked to a negative charge of another polypeptide or nucleic acid.

The agents that modulate thymic function described herein also include RNAi-inducing agents and/or RNAi molecules, e.g., an interfering RNA molecule, an shRNA, an RNAi-inducing agent, or a combination thereof. Such agents provide a portion of RNA that is complementary to a region of a target nucleic acid transcript. Essentially, these RNAi-inducing agents and/or RNAi molecules downregulate expression of the protein encoded in the target nucleic acid transcript. RNAi molecules comprise RNA or RNA-like structures typically containing 15-50 base pairs (such as aboutl8-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

RNAi molecules comprise a sequence substantially complementary, or fully complementary, to all or a fragment of a target gene. RNAi molecules may complement sequences at the boundary between introns and exons to prevent the maturation of newly-generated nuclear RNA transcripts of specific genes into mRNA for transcription. RNAi molecules complementary to specific genes can hybridize with the mRNA for that gene and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG).

RNAi molecules can be provided to the cell as “ready-to-use” RNA synthesized in vitro or as an antisense gene transfected into cells which will yield RNAi molecules upon transcription. Hybridization with mRNA results in degradation of the hybridized molecule by RNAse H and/or inhibition of the formation of translation complexes. Both result in a failure to produce the product of the original gene.

The length of the RNAi molecule that hybridizes to the transcript of interest should be around 10 nucleotides, between about 15 or 30 nucleotides, or about 15, 16, 17, 18, 19, 20, 21, 22, 23, 24, 25, 26, 27, 28, 29, 30 or more nucleotides. The degree of identity of the antisense sequence to the targeted transcript should be at least 75%, at least 80%, at least 85%, at least 90%, or at least 95.

RNAi molecules may also comprise overhangs, i.e. typically unpaired, overhanging nucleotides which are not directly involved in the double helical structure normally formed by the core sequences of the herein defined pair of sense strand and antisense strand. RNAi molecules may contain 3′ and/or 5′ overhangs of about 1-5 bases independently on each of the sense strands and antisense strands. In one embodiment, both the sense strand and the antisense strand contain 3′ and 5′ overhangs. In one embodiment, one or more of the 3′ overhang nucleotides of one strand base pairs with one or more 5′ overhang nucleotides of the other strand. In another embodiment, the one or more of the 3′ overhang nucleotides of one strand base do not pair with the one or more 5′ overhang nucleotides of the other strand. The sense and antisense strands of an RNAi molecule may or may not contain the same number of nucleotide bases. The antisense and sense strands may form a duplex wherein the 5′ end only has a blunt end, the 3′ end only has a blunt end, both the 5′ and 3′ ends are blunt ended, or neither the 5′ end nor the 3′ end are blunt ended. In another embodiment, one or more of the nucleotides in the overhang contains a thiophosphate, phosphorothioate, deoxynucleotide inverted (3′ to 3′ linked) nucleotide or is a modified ribonucleotide or deoxynucleotide.

Small interfering RNA (siRNA) molecules comprise a nucleotide sequence that is identical to about 15 to about 25 contiguous nucleotides of the target mRNA. In some embodiments, the siRNA sequence commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl:S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Pei et al. 2006, Reynolds et al. 2004, Khvorova et al. 2003, Schwarz et al. 2003, Ui-Tei et al. 2004, Heale et al. 2005, Chalk et al. 2004, Amarzguioui et al. 2004).

The RNAi molecule modulates expression of RNA encoded by a gene. Because multiple genes can share some degree of sequence homology with each other, in some embodiments, the RNAi molecule can be designed to target a class of genes with sufficient sequence homology. In some embodiments, the RNAi molecule can contain a sequence that has complementarity to sequences that are shared amongst different gene targets or are unique for a specific gene target. In some embodiments, the RNAi molecule can be designed to target conserved regions of an RNA sequence having homology between several genes thereby targeting several genes in a gene family (e.g., different gene isoforms, splice variants, mutant genes, etc.). In some embodiments, the RNAi molecule can be designed to target a sequence that is unique to a specific RNA sequence of a single gene.

For example, an RNAi molecule can be provided for a molecule that regulates processes essential for and/or involved in modulation of thymic function. Subsequent to administration of this RNAi-inducing agent, the amount of a marker of T cell exhaustion may be increased or decreased in a subject.

The agents can also include cells that have been isolated from a subject, engineered or modified so as to enable enhanced production or regulation of an agent that modifies T cell function, and reintroduced to the subject, thereby affecting a modification of T cell function in the subject. In some cases, the modified cells are autologous, provided to the subject from which they were initially isolated, whereas in other the modified cells are provided to a different subject.

Agents that modulate T cell function also include nucleic acids, e.g., gene therapy, e.g., an expressed or expressible nucleic acid encoding a protein agent described herein that modulates T cell function. Such agents are produced by conventional methods known in the art (e.g., see Templeton, Gene and Cell Therapy, 2015; Green and Sambrook, Molecular Cloning, 2012).

Other agents include agents that modulate T cell function by modifying the promotor region of at least one gene in one or more target cells. For example, the one or more target cells could comprise a T cell, whereas in other embodiments, the one or more target cells could comprise thymocytes, thymic epithelial cells, thymic stromal cells, or a combination thereof

Such agents described herein can be designed and produced by conventional methods known in the art (e.g., Templeton, Gene and Cell Therapy, 2015; Green and Sambrook, Molecular Cloning, 2012)

### Assessment of T Cell Function

Included in the invention are methods of treating a subject in need thereof with a combination of a checkpoint inhibitor (e.g., a checkpoint inhibitor described herein) with an agent that modulates T cell function (e.g., an agent that modulates T cell function described herein), wherein the combination results in synergistic improvement of one or more assessment of T cell function described below.

For example, methods described herein include the assessment of certain thymic activities, functions or characteristics. For example, methods described herein can modulate (e.g., increase or decrease, as desired) T cell exhaustion, TILs, T-cell diversity, T cell clonality, thymocytes, thymic epithelial cells, thymic stromal cells, and thymus size. Accordingly, subjects being treated may be assed for one or more such thymic activities, functions or parameter before, during and/or after treatment.

In some embodiments, subjects are assessed before treatment, e.g., to establish a baseline level of a particular thymic parameter. In some such instances, a subject may be selected for treatment based on a pre-treatment assessment described herein. In some embodiments, a subject is assessed after a first administration of the combination therapy, e.g., one or more times (e.g., 2, 3, 4, 5, 7, 10, 15 or more times) during the period encompassing the treatment regimen. In some such embodiments, a subject may be monitored for disease treatment or progression based on assessments during the period encompassing the treatment regimen, e.g., where an increase in a thymic function parameter is intended by treatment, such assessments may be used to confirm the effect of treatment, or to determine whether to stop or continue treatment. In other embodiments, a subject is assessed after the period encompassing a treatment regimen is complete. The results of such assessments may be used, e.g., to determine if the subject should undergo a different treatment, continue with another round of the same treatment regimen, or another action determined by the subject's health care provider.

In one aspect, the invention features a method of selecting a subject for treatment with an agent that modulates thymus or T-cell function (e.g., an agent that modulates thymus or T-cell function described herein) in combination with an inhibitor of immune checkpoint (e.g., an inhibitor of checkpoint described herein). The method includes

(a) identifying a subject who has a tumor or cancer;

(b) assessing (or having assessed) the subject for inadequate baseline immune function, e.g., assessing (or having assessed) a biological sample from the subject for one or more of: a marker of T cell exhaustion (e.g., a marker of T cell exhaustion described herein); a marker of T cell cytokine receptor expression (e.g., a marker of T cell cytokine expression as described herein); a marker of tumor cytokine expression (e.g., a marker of tumor cytokine expression as described herein); a tumor infiltrating lymphocyte; a marker of T cell diversity (e.g., a marker of T cell diversity described herein); a marker of T cell clonality (e.g., a marker of T cell clonality described herein); a marker of thymocytes (e.g., a marker of thymocytes described herein); a marker of thymic epithelial cells (e.g., a marker of thymic epithelial cells described herein); a marker of thymus stromal cells (e.g., a marker of thymic stromal cells described herein); a marker of CAR-T persistence and/or function (e.g., a marker of CAR-T persistence described herein); a marker of memory T cells (e.g., a marker of memory T cells described herein);

(c) selecting the subject for treatment with the agent that modulates thymus or T-cell function in combination with the inhibitor of immune checkpoint, e.g., prescribing to the subject treatment with the agent that modulates thymus or T-cell function in combination with the inhibitor of immune checkpoint based on the assessment; and

(d) optionally further treating the subject with the agent that modulates thymus or T-cell function in combination with the inhibitor of immune checkpoint.

Human colorectal tumors that express high levels of IL-7 do not respond to IL-7 therapy in combination with the immune checkpoint inhibitor anti-PD-L1 antibody in mouse models. Thus, in one embodiment, the subject is selected if the biopsied tumor expresses low IL-7 levels (e.g., 10%, 25%, 50%, 75%, 100% lower as compared to IL-7 levels in equivalent healthy tissue).

In some embodiments, the biological sample assessed is a biopsied tumor. In some embodiments, the biological sample assessed is a biopsied tumor-draining lymph node. In some embodiments, the biological sample assessed is the subject's peripheral blood. In one embodiment, the peripheral blood is withdrawn before assessment.

In some embodiments, the subject is selected if the assessed tissue or blood has T cells with high expression (e.g., 10%, 25%, 50%, 75%, 100% higher as compared to T cells from healthy tissue) of one or more (e.g., 2, 3, 4, 5, 6 or more) cytokine receptor chains that are selected from: CD127, CD25, CD122, CD124, CD122, CD360, CD132, and a combination thereof. In some embodiments, the subject is selected if the assessed tissue or blood has T cells with high expression (e.g., 10%, 25%, 50%, 75%, 100% higher as compared to T cells from healthy tissue) of the common gamma chain receptor (CD132).

In some embodiments, the subject is selected if the subject's biopsied tumor has low expression (e.g., 10%, 25%, 50%, 75%, 100% lower as compared to equivalent healthy tissue) of one or more (e.g., 2, 3, 4, 5, 6 or more) cytokines that are selected from: IL-7, IL-2, IL-4, IL-9, IL-15, IL-21, and a combination thereof. In one embodiment, the subject is selected if the subject's biopsied tumor has low expression (e.g., 10%, 25%, 50%, 75%, 100% lower as compared to equivalent healthy tissue) of IL-7.

In some embodiments, the subject is selected if the subject's biopsied tumor has a low ratio of expression of one or more (e.g., 2, 3, 4, 5, 6 or more) cytokines relative to expression of a component of the cytokine's cognate receptor on TILs present in the biopsied tumor (e.g., 10%, 25%, 50%, 75%, 100% lower relative expression of the cytokine and its cognate receptor component compared the relative expression of the same cytokine and cytokine receptor component in equivalent healthy tissue). The ratio of expression is measured using one or more (e.g., 2, 3, 4, 5, 6) pairs of cytokines and receptor components selected from: IL-7/CD127, IL-2/CD25, IL-2/CD122, IL-4/CD124, IL-15/CD122, and IL-21/CD360. In one embodiment, the subject is selected if the subject's biopsied tumor has a low ratio of expression of IL-7 relative to expression of IL-7R (CD127) on TILs present in the biopsied tumor.

T cell exhaustion: The invention provides, inter alia, methods of decreasing T cell exhaustion. T cell exhaustion, also referred to as T cell dysfunction, refers to dysfunctional state of T cells indicated by reduced or absent production of effector cytokines IFNg, TNFa, IL-2, effector molecules perforin, granzyme A, B, or K, reduced ability of cells to proliferate, and increased expression of co-inhibitory receptors PD-1, LAG-3, TIM-3, CTLA-4, CD160, 2B4 (CD244), BTLA, or TIGIT as described in the art (PMID: 26205583, PMID:21739672).

T cell exhaustion can be assessed by the following methods: T cells are stained for the expression of above mentioned surface receptors, or reactivated ex vivo with cognate peptide, PMA+lonomycin, or anti-CD3, to assess their ability to produce cytokines and other effector molecules, as well as their ability to proliferate as measured by the increase of Ki-67 expression, incorporation of BrdU, or dilution of a cell dye such as CSFE or CellTrace Violet.

In certain embodiments, T cell exhaustion is increased or decreased in a subject, as determined by these methods, by at least 5%, 10%, 15%, 20%, 25%, 30%, 35%, 40%, 45%, 50%, 55%, 60%, 65%, 70%, 75%, 80%, 85%, 90%, 95% or 98% compared to prior to treatment.

Memory T cell response:

An important benefit of the invention is to stimulate a robust memory T cell response. This memory T cell response will aid in the clearance of pathogens or tumor cells and will prevent the occurrence of relapse.

During a T cell response to a viral infection or a tumor, antigen-specific CD8 T cells will be activated by antigen-presenting cells and will differentiate into effector T cells. Effector CD8 T cells proliferate rapidly, produce pro-inflammatory cytokines, and kill infected target cells or tumor cells using cytotoxic molecules such as granzymes and perforin. The number of antigen-specific T cells responding to a pathogen peaks at day 8 after the initial infection and then the contraction phase of the T cell response begins. During contraction, the majority of T cells die and 5-10% persist as memory cells (PMID: 23080391). Before the contraction phase begins, T cells have already entered a molecular program of cellular death during the contraction phase or persist as memory cells. The antigen-specific cells that will eventually die during contraction are termed “short-lived effector cells” (SLECs) and the cells that will persist are termed “memory-precursor effector cells” (MPECs). During an infection, these cells can be differentiated using the cell surface markers KLRG1 and CD127 (IL-7Ra). SLECs will be KLRG1+ CD127− and MPECs will be KLRG1−CD127+ (PMID: 23080391). An increase in the percentage of cells in the MPEC population indicates a stronger memory T cell response and greater protective immunity.

In certain embodiments, memory T cells are increased or decreased in a subject, as determined by these methods, by at least 5%, 10%, 15%, 20%, 25%, 30%, 35%, 40%, 45%, 50%, 55%, 60%, 65%, 70%, 75%, 80%, 85%, 90%, 95% or 98% compared to prior to treatment. In some embodiments, MPECs comprise between 1-20%, between 2-20%, between 5-20%, between 2-15%, between 5-15%, between 7-20%, between 7-15%, between 10-20% of T cells in a treated subject.

Tumor infiltrating lymphocytes (TILs): Tumor infiltrating lymphocytes (TILs) are populations of immune cells that are associated with tumor tissue. TIL can be made up of lymphocytes such as CD4 T cells, CD8 T cells, γδ T cells, natural killer T (NKT) cells, and NK cells, and also myeloid cells such as macrophages, dendritic cells, and myeloid-derived suppressor cells (MDSCs). These populations of cells can be enriched from single-cell suspensions of tumor tissue using density gradient centrifugation. The presence of TILs in tumors is associated with better prognosis, particularly for CD8 T cells. TILs can be assessed, identified, and/or phenotyped by direct antibody labeling and flow cytometric analysis. CD45 is often used to identify hematopoietic cells from the tumor, which can then be phenotyped for more parameters such as CD3, CD4, CD8, CD11b, CD11c, NK1.1. Better phenotype and function of CD8 T cells in tumors is associated with good responses to immunomodulatory antibody therapy (PD-1 and CTLA-4 pathways) and can be assessed using flow cytometry markers that have been described previously (PMID: 25754329; PMID: 23197535).

In certain embodiments, TILs are increased or decreased in a subject, as determined by these methods, by at least 5%, 10%, 15%, 20%, 25%, 30%, 35%, 40%, 45%, 50%, 55%, 60%, 65%, 70%, 75%, 80%, 85%, 90%, 95% or 98% compared to prior to treatment.

### T Cell Diversity:

T cell diversity refers to the range of phenotypically, genetically, epigenetically, biochemically, and/or functionally distinct T cell populations present within a given sample or subject. T cell diversity can be assessed through a wide variety of methods known to the art. For example, T cell diversity can be assessed through qualitative and/or quantitative comparison of T cell receptor repertoire, as described herein. Importantly, T cell diversity can also be assessed by methods known to the art that do not involve and that are independent of TCR analysis. For example, high-resolution mass spectrometry can be used to map the proteome from cytotoxic T lymphocytes and use this information to identify different functional subgroups that vary in population size and cytotoxic efficacy (PMID: 26551880) Additionally, it is known to the art that engagement of different metabolic pathways and properties across subsets of T-cell populations can lead to functionally distinct T-cell populations; thus, T cell diversity can also be assessed through quantitative or qualitative assessment of such metabolic pathways and properties through a variety of methods known to the art (PMID 23746840, PMID 26261266). The diversity of cells can also be assessed from blood samples or other tissues such as biopsied tumor, using flow cytometry or mass cytometry (CyTOF), by staining cell surface markers. For example, CD4 T cells can be divided into naive (CD4+CCR7+CD45RA+CD45RO−), TH1 (CD4+CXCR3+CCR6-CD161−), TH17 (CD4+CCR6+CD161+CXCR3−), TH2 (CD4+CRTH2+CXCR3−), Treg (CD4+CD127IoCD25+), memory (CD4+CD45RA-CD45RO+), TCM (CD4+CCR7+CD45RA-CD45RO+), TEM (CD4+CCR7-CD45RA-CD45RO+), TEMRA (CD4+CCR7-CD45RA+CD45RO−), Tr1: (CD49b and Lag3 co-expression), Tfh (CXCR5, PD-1, BCL6, FoxP3−), and Tfr (CXCR5, PD-1, BCL6, FoxP3+), and CD8 T cells can be similarly divided into naive (CD8+CCR7+CD45RA+CD45RO−), TCM (CD8+CCR7+CD45RA-CD45RO+), TEM (CD8+CCR7-CD45RA-CD45RO+), TEMRA (CD8+CD45RA+CCR7-CD45RO−), Tc1 (IFNg expression following restimulation) Tc2 (CRHT2+) and Tc17 (CCR5, CCR6, CD161) as known to art (PMID: 23624599, 26146074, 25177353, 7525836, 11069080, 21706005). Following treatment with the inventions described herein, the distribution of the different subsets of these cells can be measurably altered, thereby quantitatively impacting the diversity of the T cells in the subject.

In certain embodiments, T cell diversity is increased or decreased in a subject, as determined by these methods, by at least 5%, 10%, 15%, 20%, 25%, 30%, 35%, 40%, 45%, 50%, 55%, 60%, 65%, 70%, 75%, 80%, 85%, 90%, 95% or 98% compared to prior to treatment.

### TCR Repertoire Diversity:

TCR repertoire diversity refers to the number of different T cell receptors (TCR) in a population of T cells; sources of diversity within TCR repertoire include unique alpha and beta TCR subunits, genetic differences stemming from V-J (TCR alpha) and V-D-J (TCR beta) recombination, as well as different terminal deoxynucleotidyl transferase (TdT) - introduced nucleotides at DNA junctions, resulting in sequence variation. (Murphy et al., Janeway's Immunobiology, 8th Edition, 2012). Because T-cells expand clonally, in some cases a given TCR can be shared by multiple T-cells that have expanded from a common parent cell; in this situation and in similar situations, TCR repertoire diversity can additionally refer to the number of different clonal populations of T-cells that harbor distinct TCRs within a sample. An increase in TCR repertoire diversity is an indicator of a robust antigen-specific T cell response to a pathogen or tumor, as increased diversity suggests that more T cells are capable of responding to the pathogen or tumor, which is beneficial for ensuring effective control of the pathogen or tumor. Increases in repertoire diversity also suggest an environment more favorable for effective immunity, as subdominant clones with weaker TCR affinity for their antigen are also able to become activated and proliferate.

TCR repertoire diversity can be assessed by methods known to art including RNA-Sequencing, DNA-sequencing, TCR-targeted sequencing, and TCR probe-based PCR. For example, TCR repertoire profiling can measure the distribution of individual T cell clones in a population. This is done by amplifying the CDR3 region of the TCR alpha and TCR beta chains from T cell genomic DNA and next-generation sequencing the region. Since each clonal population of T cells has a unique T cell receptor, analysis of deep sequencing data from a TCR repertoire profile can yield information about the total number of different T cell clones and T-cell receptors found in the population. Both of these metrics can be measured from a single sample, given that the sample is sequenced deeply enough and the amount of input T cell genomic DNA is standardized. (US 20100035764, PMID: 26404496, PMID: 2343517, PMID: 24329790).

In certain embodiments, T cell repertoire diversity is increased or decreased in a subject, as determined by these methods, by at least 5%, 10%, 15%, 20%, 25%, 30%, 35%, 40%, 45%, 50%, 55%, 60%, 65%, 70%, 75%, 80%, 85%, 90%, 95% or 98% compared to prior to treatment.

T Cell Clonality:

T cell clonality refers to the absolute or relative size of a clonal T cell population or of several clonal T cell populations, including peripheral blood mononuclear cells, TILs, or other tissue-derived immune cells, or the magnitude of a T cell expansion giving rise to said clonal population. For example, during the course of a response to a particular antigen, a T cell that is specific for said antigen will undergo a series of receptor-mediated reactions, resulting in the division of the cell into daughter cells and establishing aclonal population of T cells expressing the same TCR. In general, an increase TCR clonality is a good indicator of a robust antigen-specific T cell response to a pathogen or tumor. The term “T cell clonality” can be used to refer to the absolute or relative size of a specific T cell clonal population, or it can be used to refer to multiple clonal T cell populations simultaneously. Increases in T cell clonality can also suggest that T cells are expanding vigorously after antigen recognition, indicating that they are not functionally suppressed (e.g., exhausted).

T cell clonality can be assessed by performing T cell spectratyping on a population of T cells, performing tetramer staining on a population of T cells, sequencing at least one TCR subunit from a population of cells, staining a population of T cells with an anti-TCR antibody, performing flow cytometry, or a combination thereof. TCR sequence can be assessed by methods known to art including RNA-Sequencing, DNA-sequencing, TCR-targeted sequencing, and TCR probe-based PCR (US 20100035764 Al, PMID: 26404496, PMID: 2343517, PMID: 24329790).

In certain embodiments, T cell clonality is increased or decreased in a subject, as determined by these methods, by at least 5%, 10%, 15%, 20%, 25%, 30%, 35%, 40%, 45%, 50%, 55%, 60%, 65%, 70%, 75%, 80%, 85%, 90%, 95% or 98% compared to prior to treatment.

Thymocytes:

Thymocytes are differentiating T-cells and T-cell progenitors found in the thymus. In the thymus, under the influence of the thymic stromal microenvironment, immature thymocytes acquire various cell surface molecules useful in their future role as mature T cells. Thymocytes can be assessed by multiple methods as described in the art. For example, thymocytes can be assessed by assaying for cell surface expression of developmentally regulated thymocyte markers, using labeled antibodies that specifically bind to these markers. For example, the most immature CD4−CD8− double-negative (DN) thymocytes give rise to CD4+CD8+ double-positive (DP) thymocytes, which give rise to mature CD4+CD8− single-positive (SP) and CD4−CD8+ SP T cells. The DN population can be further subdivided by the expression of CD44 and CD25: CD44+CD25− (DN1) cells differentiate into CD44+CD25+ (DN2) cells, which give rise to CD44−CD25+ (DN3) cells, which finally become the most mature CD44−CD25− (DN4) DN population. The DN4 cells may pass through an intermediate population expressing either coreceptor alone before becoming DP cells. This intermediate population, most commonly expressing CD8, is known as immature single positive (ISP) cells. Progression beyond the DN3 stage is dependent on successful rearrangement of a TCRβ-chain gene and pre-TCR signaling, whereas differentiation from DP to mature SP cell is dependent on the expression and positive selection of an αβTCR (Von Boehmer et al., Immunol. Rev. 191: 62, 2003; Ceredig and Rolink, Nat. Rev. Immunol. 2: 888, 2002). Additionally, the cellularity of the thymus (including thymocytes) can be assessed using clinical imaging modalities such as MRI, CT, or PET, as described in the art (Brink et al., J. Nuc. Med., 2001; Ackman and Wu, Am. J. Roent., 2011).

In certain embodiments, thymocytes (e.g., one or more of: CD4+CD8+ DP thymocytes; CD4+CD8− SP thymocytes; CD4-CD8+ SP T cells; CD44+CD25− DN1 cells; CD44+CD25+ DN2 cells; to CD44−CD25+ DN3 cells; CD44−CD25− DN4) are increased or decreased in a subject, as determined by these methods, by at least 5%, 10%, 15%, 20%, 25%, 30%, 35%, 40%, 45%, 50%, 55%, 60%, 65%, 70%, 75%, 80%, 85%, 90%, 95% or 98% compared to prior to treatment.

Thymic Epithelial Cells:

Thymic epithelial cells or TECs are epithelial cells located in the thymus, including cortical thymic epithelial cells (cTECs) and medullary TECs (mTECs). They comprise the stromal compartment of the thymus responsible for guiding developing thymocytes through various developmental stages and to maturity. Thymocytes can be assessed by multiple methods as described in the art. For example, TECs can be assessed by assaying for cell surface expression of developmentally regulated TEC markers using labeled antibodies that specifically bind these markers; such cell surface markers include Keratin 8 (K8), K5, EpCAM, MHC class I, MHC class II, CD45, CD80, CD86, CD90, CD11c, CCL25, RANK, RANKL and CXCL12 (Gray et al., Immun. Meth., 2008). Other markers of TECs are intracellular or secreted and can be assessed using intracellular staining or other methods known to the art. Such markers include AIRE, FezF2, FoxN1, Hoxa3, proteasome subunit 135t, proteasome subunit 135t, BMP4, retinoic acid, Wnt, Shh, FGF, and SCF. In some case the absence of a particular marker can be useful in assessing the identity of a cell as a TEC, for example, CD45. Additionally, the cellularity of the thymus (including thymic epithelium) can be assessed using clinical imaging modalities such as MRI, CT, or PET, as described in the art (Brink et al., J. Nuc. Med., 2001; Ackman and Wu, Am. J. Roent., 2011).

In certain embodiments, thymic epithelial cells are increased or decreased in a subject, as determined by these methods, by at least 5%, 10%, 15%, 20%, 25%, 30%, 35%, 40%, 45%, 50%, 55%, 60%, 65%, 70%, 75%, 80%, 85%, 90%, 95% or 98% compared to prior to treatment.

Thymic Stromal Cells:

Thymic stromal cells are the non-thymocyte cells within the thymus that mediate the thymus's system for ‘training’ T lineage progenitor T cells as developing thymocytes for proper binding of a T cell receptor recognizing ‘self’ in the context of a peptide; this compartment includes fibroblasts, epithelium, endothelium (Gray et al., J Immun., 2007). The main stromal compartments responsible for guiding developing thymocytes are lined with TECs. Among other cell types, thymic stromal cells also comprise fibroblasts; fibroblasts are also known to play a role in guiding thymocyte development. Thymic stromal cells are heterogeneous and can be assessed by determining the relative or absolute quantity of the subsets comprising thymic stromal cells (e.g., fibroblasts, TECs, endothelium). For example, the stromal cell subset comprising thymic fibroblasts can be assessed through the use of the monoclonal antibody MTS-15 as known to the art (Gray et al., J. Immun., 2007).

In certain embodiments, thymic stromal cells (e.g., one or more or all of: fibroblasts, TECs, endothelium) are increased or decreased in a subject, as determined by these methods, by at least 5%, 10%, 15%, 20%, 25%, 30%, 35%, 40%, 45%, 50%, 55%, 60%, 65%, 70%, 75%, 80%, 85%, 90%, 95% or 98% compared to prior to treatment.

Thymus Size:

Generally speaking, the size and cellular composition of a human thymus is proportional to its output and level of activity. Thymus size can be assessed by direct visualization in vivo, ex vivo, or intraoperatively, as well as through a wide variety of histological processing techniques known to the art, including but not limited to hematoxylin and eosin staining. Thymus size can also be assessed using biomedical imaging techniques such as magnetic resonance imaging (MRI or MR imaging), computed tomography (CT), and position emission tomography (PET) (PMID: 21700977). Recently, 18F-FDG PET imaging is an important tool for the visualization and staging of human cancer. In infants and young adults, the thymus's extensive cellularity and metabolic activity results in physiologic uptake of FDG, although this disappears during adolescence as the thymus involutes. (PMID 8896924) In contrast, transient thymic hyperplasia has been sporadically observed following chemotherapy, particularly for testicular carcinoma or malignant lymphoma; additionally, a retrospective study showed that in a subset of adult subjects who experience chemotherapy-induced thymic hyperplasia, the hyperplasia is detectible via increased FDG uptake, rendering thymus FDG uptake an imaging biomarker for enhanced thymic cellularity (PMID 11337547).

In certain embodiments, thymus size is increased or decreased in a subject, as determined by these methods, by at least 5%, 10%, 15%, 20%, 25%, 30%, 35%, 40%, 45%, 50%, 55%, 60%, 65%, 70%, 75%, 80%, 85%, 90%, 95% or 98% compared to prior to treatment.

Cancer immunogenicity

In some aspects of the invention, a subject having a low, moderate or highly immunogenic cancer is treated with methods of the invention. Immunogenicity of tumors is known to increase with the mutational burden of the respective tumor (PMID: 25594174, 26028255, 25765070); thus, the methods may include categorization of the tumor or cancer into highly mutated (over 50 somatic mutations per 1,000,000 bases), moderately mutated, (between 0.5 and 50 somatic mutations per 1,000,000 bases), and low-mutated tumors (<0.5 somatic mutations per 1,000,000 bases), which correspond to high, moderate, and low immunogenic tumors, respectively.

In one embodiment, tumor mutational landscape is determined by DNA sequencing of the biopsied tumor tissue and comparison of the DNA sequence obtained to that of somatic DNA samples obtained from the same subjects. In another embodiment, total mRNA from a biopsied tumor is sequenced using, e.g., a cDNA microarray, and the results are analyzed to determine the abundance of immune signatures associated with immunogenicity through methods known to art (e.g., through the use of a method such as Cell type Identification By Estimating Relative Subsets Of known RNA Transcripts (CIBERSORT)). (PMID: 26193342, 25822800). For example, total mRNA from a biopsied tumor can be isolated and the relative abundance of key immune genes, such as CD3, CD8a, GZMB, IFNG and others, can be assessed (PMID: 25594174). High relative cumulative abundance of these transcripts provides for the characterization of the tumor as highly immunogenic, whereas low abundance or absence of these transcripts provides for characterization of the tumor as poorly immunogenic.

In some embodiment, the tumor is biopsied and the tissue examined for the immune contexture and the numeration of two lymphocyte populations (CD3/CD45RO, CD3/CD8 or CD8/CD45RO), both in the core of the tumor and in the invasive margin of the tumors, a measure known to art as Immunoscore (PMID: 24122236, 23579076, 23890060, 24138885, 17008531). Immunoscore grades tumors on a 10-14 scale, where 14 is defined as highly immune, 11-13 as moderately immune, and 10 as poorly immunogenic tumors.

In some embodiments, tumor-infiltrating lymphocytes are assessed (e.g., number, activity) in the tumor, one or more margins of the tumor, tissue adjacent to the tumor, and one or more draining lymph nodes. In another embodiment, tumor-infiltrating lymphocyte grade is assessed and ranked on a four-tiered TIL grading scheme (0 to 3), based at least in part on assessment of TIL density (mild, moderate, or marked) and distribution (focal, multifocal, or diffuse) across the entire extent of biopsied tumor, as known to art (PMID: 22711850). Grade 3 tumors are defined as highly immunogenic, grade 1 and 2 tumors are defined as moderately immunogenic, and grade 0 tumors are defined as poorly immunogenic tumors.

In some embodiments, memory T cells are assessed in the tumor (e.g., number present, antigen specificity/diversity) in the tumor, one or more margins of the tumor, tissue adjacent to the tumor, and one or more draining lymph nodes.

In some embodiments, T cell populations (e.g., memory, helper, cytotoxic, regulatory) are assessed (e.g., number present, ratio, diversity, auto-reactivity) in the tumor, one or more margins of the tumor, tissue adjacent to the tumor, and one or more draining lymph nodes.

In some embodiments, B cell populations are assessed (e.g., number present, ratio to other immune cells, diversity, auto-reactivity) in the tumor, one or more margins of the tumor, tissue adjacent to the tumor, and one or more draining lymph nodes.

Inhibitors of Immune Checkpoint

Described herein are methods of using checkpoint inhibitors in therapeutic combination with agents that affect thymic function. Checkpoint inhibitors can be broken down into at least 4 major categories: i) antibodies that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, and KIR), ii) antibodies that activate stimulatory pathways directly on T cells or NK cells (e.g., antibodies targeting OX40, GITR, and 4-113B), iii) antibodies that block a suppressive pathway on immune cells or relies on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, and Ly6G), and iv) antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, and MUC1).

Such agents described herein can be designed and produced, e.g., by conventional methods known in the art (e.g., Templeton, Gene and Cell Therapy, 2015; Green and Sambrook, Molecular Cloning, 2012).

### Chronic Infections

Described herein, inter alia, are methods of treating a chronic infection in a subject. Generally, persistent infection is caused by a pathogen from one of the 3 major categories:

i) viruses, including the members of the Retroviridae family such as the lentiviruses (e.g. Human immunodeficiency virus (HIV) and deltaretroviruses (e.g., human T cell leukemia virus I (HTLV-I), human T cell leukemia virus II (HTLV-II)); Hepadnaviridae family (e.g. hepatitis B virus (HBV)), Flaviviridae family (e.g. hepatitis C virus (HCV)), Adenoviridae family (e.g. Human Adenovirus), Herpesviridae family (e.g. Human cytomegalovirus (HCMV), Epstein-Barr virus, herpes simplex virus 1 (HSV-1), herpes simplex virus 2 (HSV-2), human herpesvirus 6 (HHV-6), varicella-zoster virus), Papillomaviridae family (e.g. Human Papillomavirus (HPV)), Parvoviridae family (e.g. Parvovirus 1319), Polyomaviridae family (e.g. JC virus and BK virus), Paramyxoviridae family (e.g. Measles virus), Togaviridae family (e.g. Rubella virus) as well as other viruses such as hepatitis D virus;

ii) bacteria, such as those from the following families: Salmonella (e.g. S. enterica Typhi), Mycobacterium (e.g. M. tuberculosis and M. leprae), Yersinia (Y. pestis), Neisseria (e.g. N. meningitides, N. gonorrhea), kholderia (e.g. B. pseudomallei), Brucella, Chlamydia, Helicobacter, Treponema, Borrelia and Pseudomonas; and

iii) parasites, such as Leishmania, Toxoplasma, Trypanosoma, Plasmodium, Schistosoma, or Encephalitozoon.

### Administration

The agents (e.g., agents that modulate thymus or T-cell function) described herein may be administered to a subject by various routes including, for example, orally or parenterally (e.g., intravenously, intramuscularly, subcutaneously, intraorbitally, intracapsularly, intraperitoneally, intrarectally, intracisternally, intratumorally, intravasally, intradermally. The agents may be administered to the site of a target tissue, for example, intravenously or intra-arterially into a blood vessel supplying a tumor.

The agents described herein are administered as a combination therapy regimen, either sequentially or concurrently. Each agent may be administered a single dose, either as a bolus or by infusion over a relatively short period of time, or through a fractionated treatment protocol, in which multiple doses are administered over a prolonged period of time. One or both agents of the combination may be administered in a controlled release formulation.

A particular combination therapy treatment regimen described herein will typically define the doses and periodicity of administration of each agent such that the effects of the separate agents on the subject overlap and in some cases synergize. In some embodiments, the agent that modulates thymic function described herein (e.g., IL-7 or a fragment or derivative thereof) is administered before the checkpoint inhibitor (e.g., 12 hours, 1 day, 2 days, 3 days, 4 days, one week, two weeks before). In some embodiments, the subject is assessed for the level of the agent that modulates thymic function (e.g., IL-7) at periodic times during the treatment regimen, e.g., to ensure that the agent is present at a threshold level over the course of the combination therapy. In one example, the agent is administered in a controlled release formulation. The overall period of time over which a particular combination therapy treatment regimen is followed by a subject may vary depending on the response and health of the subject but typically is at least one month, 6 weeks, 2 months, 3 months, 6 months, 9 months, a year, 18 months, 2 years or more.

In certain embodiments, the agents described herein are administered in doses of 0.01-10 mg/kg, 0.05-5 mg/kg, 0.1-5 mg/kg, 0.2-5 mg/kg, 0.5-5 mg/kg, 0.5-1 mg/kg, 0.5-5 mg/kg, 0.5-10 mg/kg, 1-10 mg/kg, 1-5 mg/kg, or any combination thereof.

In some embodiments, the invention features a method comprising administering a checkpoint inhibitor (e.g., a checkpoint inhibitor described herein) in combination with an agent that modulates T cell function (e.g., an agent that modulates T cell function described herein), and assessing the subject for an adverse symptom or condition described herein.

In some embodiments, administration of the composition may increase the subject's risk of developing an auto-immune disease, e.g., multiple sclerosis, rheumatoid arthritis, systemic lupus erythematosus, scleroderma, psoriasis, celiac disease, vitiligo, Hashimoto's disease (autoimmune thyroiditis), Addison's disease, Grave's disease, Sjogren's syndrome, or type 1 diabetes.

In some embodiments, administration of the composition in subjects with a pre-existing auto-immune disease, e.g., multiple sclerosis, rheumatoid arthritis, systemic lupus erythematosus, scleroderma, psoriasis, celiac disease, vitiligo, Hashimoto's disease (autoimmune thyroiditis), Addison's disease, Grave's disease, Sjogren's syndrome, or type 1 diabetes, may increase the subject's risk of experiencing one or more adverse disease-associated symptoms.

In one embodiment, administration of the composition in subjects with pre-existing rheumatoid arthritis disease may increase the subject's risk of experiencing one or more adverse disease-associated symptoms (e.g., increased inflammation in the synovial membrane of affected joints).

In one embodiment, administration of the composition in subjects with pre-existing multiple sclerosis disease may increase the subject's risk of experiencing one or more adverse disease-associated symptoms (e.g., increased relapse rate, increased optic neuritis, reduced ability to keep balance and walk).

In one embodiment, administration of the composition in subjects with pre-existing psoriasis may increase the subject's risk of experiencing one or more adverse disease-associated symptoms (e.g., increased skin redness, increased skin irritation, increased surface area of thick, flaky, and/or silver-white patches of skin).

In one embodiment, administration of the composition in subjects with pre-existing celiac disease may increase the subject's risk of experiencing one or more adverse disease-associated symptoms (e.g., increased abdominal bloating and pain, increased frequency and/or severity of diarrhea, increased fatigue, increased dermatitis herpetiformis).

In some embodiments, administration of the composition may increase the subject's risk of experiencing one or more immune-related symptoms, e.g., immune-mediated endocrinopathy, immune-mediated pneumonitis, immune-mediated colitis, immune-mediated hepatitis, immune-mediated nephritis and renal dysfunction, immune-mediated skin adverse reactions, encephalitis, or complications of an allogeneic hematopoetic stem cell therapy.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Example 1

### IL-7/Anti-PD-1 Therapy in Human Chronic Infection

Patients with confirmed HIV on HAART and on stable regimen for at least 3 months prior to enrollment are separated into 2 groups of 35. The first group is maintained on HAART therapy with addition of placebo and the second group of patients is given 3 cycles, separated by 2 months, where each cycle constitutes 3 doses of 20 μg/kg/week recombinant human IL-7 (rhlL-7) and 3 mg/kg of anti-PD-1 monoclonal antibody through an intravenous infusion over 1 hour.

Blood is collected before and after the therapy to assess viral burden and immune function. HIV viral load during chronic infection is measured using the Roche Amplicor version 1.5 assay. Peripheral blood mononuclear cells (PBMC) are isolated using Ficoll gradient. A portion of the cells is used for direct staining with cell surface markers CD3, CD8, CD4, CD45RA, CD45RO, CCR7, CD62L, CD107A, PD-1, CD39, LAG-3, TIM-3, CD160, CD244, with tetramers for GP33-41 and GP276-286, and NP396-404 and intracellular transcription factors T-bet, Eomesodermin, and FoxP3, and analyzed on BD Fortessa flow cytometer (PMID: 26485519). Additional PBMC are plated in a 96-well plate in the presence of 1:1000 GolgiPlug (BD Biosciences) in the presence of HIV Gag peptide pool (1 μg ml-1 per peptide), optimal HIV peptide or without a peptide, for 4 hours (PM ID: 20890291). The cells are stained for surface antigens CD4, CD8, CD3, CD44, fixed and permeabilized and stained for intracellular cytokines TNFa, IFNg, and IL-2 as described before (PMID: 26485519).

Additionally, symptoms for Progressive Multifocal Leukoencephalopathy (PML) in HIV are closely monitored and if PML is suspected, JCV-PCR is performed on cerebrospinal fluid. (http://emedicine.medscape.com/article/1167145-overview#a6)

Patients treated with the combination therapy exhibit as lower expression of exhaustion markers and coinhibitory molecules PD-1, CD39, CD160, LAG-3, TIM-3, and TIGIT, suggesting reversal of CD4+ and CD8+ T cell exhaustion. Additionally, protective cells bearing a high expression of transcription factor T-bet relative to Eomesodermin is observed, indicating transformational shifts of exhausted cells toward regaining function. When CD4+ or CD8+ T cells are activated with viral peptides, cells from treated patients are more functional than control cells as measured by the produced effector cytokines IFNg and TNFa. Additionally, treated patients present decreased development of PML, a neurodegenerative disease associated with JC virus in HIV patients. Treated patients who do develop PML exhibit a decreased progression of the disease with fewer symptoms or decreased severity of symptoms (which may include some degree of mental impairment, vision loss, speech disturbances, facial drooping, weakness, problems with coordination, gait, sensory loss, and seizures).

### Example 2

### IL-7/Anti-PD-1 Therapy in Mouse Model of Chronic Infection

C57BL6 mice are infected with Clone 13 strain of Lymphocytic choriomeningitis virus (LCMV) as described previously (PMID: 23159438). At 30+ days following infection, the mice are treated with or without 100 ug anti-PD-1 (clone 29F.1A12) and/or 5 ug of recombinant human IL-7 intraperitoneally; 5 doses given every 2 days. The mice are sacrificed 3 days following final injection. Organs (kidney, liver, brain, and blood plasma) are collected to assess viral titer using plaque assay in Vero cells as described previously. (PMID: 21623380). Spleens are homogenized into single cell suspension. A portion of the cells is used for direct staining with cell surface markers CD3, CD8, CD4, CD44, CCR7, CD62L, CD107A, PD-1, CD39, LAG-3, TIM-3, CD160, CD244, with tetramers for GP33-41 and GP276-286, and NP396-404 and intracellular transcription factors T-bet, Eomesodermin, and FoxP3, and analyzed on BD Fortessa flow cytometer using methods known to art (PMID: 26485519). Additional splenocytes are plated in a 96-well plate in the presence of 1:1000 GolgiPlug (BD Biosciences) in the presence of viral peptides GP33-41 or GP276-286 or NP396-404 or without a peptide, for 4 hours. The cells are stained for surface antigens CD4, CD8, CD3, CD44, fixed and permeabilized and stained for intracellular cytokines TNFa, IFNg, and IL-2 as described before (PMID: 26485519)

Mice treated with the combination therapy exhibit reversal of T cell exhaustion as indicated by lower expression of exhaustion markers and coinhibitory molecules PD-1, CD39, CD160, LAG-3, TIM-3, and CD44. Additionally, treated animals have a higher proportion of protective cells bearing a high expression of transcription factor T-bet relative to Eomesodermin, further indicating the transformational shifts of exhausted cells toward regaining function. When T cells are activated with viral peptides, cells from treated animals are more functional as measured by the produced effector cytokines IFNg and TNFa.

### Example 3

### IL-7/Anti-PD-1 Therapy to Improve Human Thymic Function in Cancer Patients

Patients with a diagnosis of measurable, unresectable, stage III or IV melanoma with a life expectancy of at least 4 months are randomized into groups receiving nivolumab alone or nivolumab plus CYT107 (rhlL-7). Nivolumab is administered every two weeks as in (PMID: 26406148) and CYT107 is administered at 20 ug/kg/week.

The primary endpoint is overall survival, defined as the time from randomization to the date of death. Secondary endpoints are progression-free survival, objective response rate and partial and complete response rates. Scientific objectives of the trial include determining the association between therapy and the number, phenotype, functional capacity and clonotypic diversity of T lymphocytes in both tumor and blood samples. In addition, thymic output is monitored in patients by quantification of T cell receptor excision circles (TRECs) in peripheral blood.

Consenting patients are biopsied before the onset of treatment and two weeks after treatment begins. When possible, a third biopsy is taken at the first sign of disease progression. Biopsies are taken as described in (PMID: 20818844; PMID: 25428505). In short, biopsies are taken from cutaneous lesions and when possible, no individual lesion will be biopsied more than once. Leukocytes are isolated from biopsied tissue and immediately cryopreserved as described in (PMID: 21555851). In short, mononuclear cells are isolated by density gradient and then cryopreserved in RPMI 1640 with 40% FCS and 10% DMSO. Blood is collected from patients at the time of tumor biopsy and a portion is saved for multiplexed serum cytokine detection. For the remaining sample, mononuclear cells are immediately isolated using a Ficoll gradient and then cryopreserved in RPMI 1640 with 40% FCS and 10% DMSO.

Half of the lymphocytes from each biopsy and blood sample are used for TREC quantification, TCR deep sequencing and clonotype diversity analysis as previously described (PMID: 25428505; PMID: 25754329). Genomic DNA from blood and tumor samples will be isolated using the QiaAMP DNA blood mini kit (Qiagen). The diversity and clonality of the TCR repertoire is assayed by sequencing the CDR3 region of the TCR beta chain (Adaptive Biotechnologies). TREC quantification is done using qPCR for T cell receptor excision circles as described (PMID: 25549107) to monitor thymic output.

The combination of CYT107 and nivolumab increases the clonality of T cells infiltrating the tumor after the onset of treatment to a greater extent than nivolumab alone, suggesting an increased expansion and survival of antigen-specific clones that can control tumor growth. In paired patient biopsies from pre- and post-treatment, an expansion of the most abundant T-cell clones present before treatment occurs after the onset of combination therapy. This is consistent with the idea that CYT107 therapy reduces the dysfunction of antigen-specific T cells and allows them to proliferate more robustly (PM ID: 21295337; PMID: 19396174). Additionally, an increase in the total diversity of TCRs in the TIL sample is also observed. This increase in diversity is higher in patients receiving nivolumab and CYT107 compared to controls. From peripheral blood, the TCR repertoire diversifies after the onset of combination therapy with nivolumab and CYT107, suggesting an increase in thymic output; additionally, an increase in TREC abundance is observed in the combination therapy treated patients after the onset of treatment.

The remaining leukocytes from the tumor biopsy are used for immunophenotyping. A portion of the cells are directly stained with CD45, CD3, CD4, CD8a, CD44, PD-1, TIM-3, LAG-3, FoxP3, T-bet, and Eomesodermin. The remaining leukocytes are plated in 96-well dishes and stimulated in PMA-lonomycin in the presence of 1:1000 GolgiPlug (BD Biosciences) and stained for CD3, CD8, CD4, CD44, fixed and permeabilized, and stained for the intracellular cytokines IFNg and TNFa as described (PMID: 23197535).

TILs isolated from patients receiving combination therapy appear less exhausted as indicated by lower cell surface expression of PD-1, LAG-3 and TIM-3 than TIL from patients on nivolumab alone. Additionally, in paired patient samples, a reduction in the number of PD-1, LAG-3 and TIM-3 expressing CD8 T cells is observed after the onset of treatment. Additionally, biopsies from patients treated with combination therapy contain a greater number of CD44+ PD-1+ CD8 T cells and fewer FoxP3+ CD4 T cells as a percentage of CD45+ cells than biopsies from patients treated with nivolumab alone. An expansion of CD4+, FoxP3- T cells is also observed. The increase in CD8+ CD44+ PD-1+ T cells and decrease in CD4+ FoxP3+ T cells is evident in paired patient biopsies. CD8 T cells from combination therapy-treated mice also have greater expression of T-bet and lower expression of Eomesodermin, indicating a less terminally exhausted phenotype and greater functional capacity. This observation is also evident in paired patient biopsies, indicating a rescue of exhausted T cells within the same patient by treating with nivolumab and CYT107. Finally, both CD4 and CD8 T cells isolated from patients receiving combination therapy appear more functional, indicated by an increased potential for the production of the cytokines IFNg and TNFa.

The remaining blood samples are used for serum cytokine detection using the Luminex cytokine human 25-plex panel (Invitrogen). Plasma is isolated from blood by centrifugation at 3000 rpm for 10 min at 4 C. Serum levels of the cytokines GM-CSF, IFNs, IFNg, IL-1RA, IL-1b, IL-2, IL-2R, IL-4, IL-5, IL-6, IL-7, IL-8, IL-10, IL-12 (p40/p70), IL-13, IL-15, IL-17, and TNFa, the chemokines Eotaxin, IP-10, MCP-1, MIG, MIP-1a, MIP-1b, and RANTES are assayed by multiplex luminex assay.

The addition of CYT107 to nivolumab causes an increase in serum levels of IL-6, IL-7, IL-12, IL-17, IL-1RA, IL-1b, IFNa, IFNg, and MIP1a, but a decrease in TNFa. This observation is seen in paired patient blood samples, indicating that the increase in serum cytokine levels happen as a result of CYT107 treatment. These serum cytokines indicate a pro-inflammatory functional immune response.

### Example 4

### IL-7/Anti-PD-1 Therapy in Mouse Tumor Model to Improve TIL and T Cell Clonality

To determine the capacity of CYT107 (rhlL-7) to limit T cell exhaustion and enhance the function and expansion of T cells in the tumor, profiling of T cells isolated from transplantable mouse tumors is conducted. Four syngeneic mouse tumor models are used: MC38 and B16-F10 are implanted into C57BL/6J mice and CT26 and 4T1 are implanted into Balb/c mice. Mice are treated with either PD-1 alone (clone 29f.1a12; 100 ug i.p.) or PD-1+ CYT107 (5 ug i.p.) on a schedule that is dependent on the cell line (see table below).

| Cell line | Mouse strain | Cell injection dose | Treatment schedule |
|---|---|---|---|
| MC38 | C57BL/6J | 1M | Days 9, 12, 15 |
| B16 | C57BL/6J | 500K | Days 6, 9, 12, 15 |
| CT26 | Balb/c | 500K | Days 6, 9, 12, 15 |
| 4T1 | Balb/c | 500K | Days 3, 6, 9, 12, 15 |

Three cohorts of mice are used: The first cohort of mice (10 mice/group) is used to assess tumor progression over time. Tumor measurements are taken every 3 days for the duration of the experiment (up to 45 days).

Mice treated with the combination of anti-PD-1 and CYT107 have an increased ability to control tumor progression than mice treated with anti-PD-1 alone, as determined by a reduced tumor burden after initiation of therapy. Cures are observed in a higher percentage of mice bearing established MC38 and CT26 tumors that receive combination therapy, while cures are less common in mice receiving anti-PD-1 alone using this treatment regimen. Additionally, mice receiving combination therapy clear more established B16 melanomas and significantly improve the survival time of mice bearing 4T1 breast cancers.

A second cohort of mice (5 mice/group) is sacrificed at day 18 for immune-phenotyping of TILs. Due to the fact that cures are observed in the MC38 and CT26 tumor-bearing mice that receive combination therapy, therapy is initiated later to prevent tumor rejection and allow for TIL isolation. For this cohort, tumors are removed, digested in collagenase/DNase, and TILs are enriched on a Ficoll gradient (PM ID: 23752227). TILs are then directly stained with CD45, CD3, CD4, CD8a, CD44, PD-1, TIM-3, LAG-3, FoxP3, T-bet, and Eomesodermin. The remaining TIL are plated in 96-well dishes and stimulated in PMA-lonomycin in the presence of 1:1000 GolgiPlug (BD Biosciences) and stained for CD3, CD8, CD4, CD44, fixed and permeabilized, and stained for the intracellular cytokines IFNg and TNFa as described (PMID: 23197535).

TILs isolated from mice receiving combination therapy are less exhausted as indicated by lower cell surface expression of PD-1, LAG-3 and TIM-3. Additionally, tumors treated with combination therapy contain a greater number of CD44+ PD-1+ CD8 T cells and fewer FoxP3+ CD4 T cells as a percentage of CD45+ cells than tumors treated with anti-PD-1 alone. An expansion of CD4+, FoxP3− T cells is also observed. CD8 T cells from combination therapy-treated mice also have greater expression of T-bet and lower expression of Eomesodermin, indicating a less terminally exhausted phenotype and greater functional capacity. Finally, both CD4 and CD8 T cells isolated from mice receiving combination therapy appear more functional, indicated by an increased potential for the production of the cytokines IFNg and TNFa.

A third cohort of mice (5 mice per group) is used to sequence the CD4 and CD8 T cell repertoire of TIL isolated from tumors of mice treated with either anti-PD-1+ CYT107 or anti-PD-1 alone. For TCR repertoire profiling, mice are sacrificed at day 18 and TIL are isolated from tumors as described previously (PM ID: 23752227) and then enriched for CD4 or CD8 T cells using CD4 or CD8 negative selection MACS columns (Miltenyi). Genomic DNA is isolated from pooled CD4 or CD8 TIL from each group using the QiaAMP blood DNA mini kit (Qiagen) and the diversity and clonality of the TCR repertoire is assayed by sequencing the CDR3 region of the TCR beta chain (Adaptive Biotechnologies).

The addition of CYT107 to anti-PD-1 immunotherapy increases the clonality of CD4 and CD8 T cells infiltrating the tumor, suggesting an increased expansion of antigen-specific T cells clones that can control or reduce tumor growth.

From the same cohort of mice, serum is analyzed for cytokine, chemokine, and growth factor levels using the Luminex cytokine mouse 20-plex panel (Invitrogen). Prior to tumor harvest, plasma is collected by anesthetizing mice with ketamine/xylazine and bleeding mice by cardiac puncture (PMID: 21350616). Plasma is isolated by centrifugation of blood at 3000 rpm for 10 min at 4 C. Serum levels of the cytokines GM-CSF, IFNg, IL-1a, IL-1b, IL-2, IL-4, IL-5, IL-6, IL-10, IL-12 (p40/p70), IL-13, IL-17, and TNFa, the chemokines IP-10, KC, MCP-1, MIG, and MIP-la, and the growth factors FGF and VEGF are assayed by multiplex luminex assay (PMID: 24648449).

CYT107 therapy causes an increase in serum levels of IL-6, IL-12, IL-17, IL-1a, IL-1b, IFNg, and MIP1a, but a decrease in TNFa. These serum cytokines indicate a pro-inflammatory functional immune response and agree with previously published observations (PMID: 21295337).

### Example 5

### IL-7/Anti-PD-1 Therapy to Improve Memory Response to Cancer

Mouse Experiments:

To determine the capacity of CYT107 (rhlL-7) to enhance the memory T cell response to cancer in conjunction with anti-PD-1 immunotherapy, mice are implanted with either MC38 or CT26 tumor lines engineered to express the model antigen ovalbumin (OVA). The expression of OVA allows for antigen-specific T cells to be identified using OVA-tetramer (MBL international). Mice are divided into four treatment groups that are summarized below and treated with either anti-PD-1 (29f.1a12) or isotype control with or without CYT107. On day 18 after tumor challenge, mice are sacrificed, tumors are harvested and digested in collagenase/DNase, and TILs are enriched on a Ficoll gradient (PMID: 23752227). TILs are then directly stained for CD45, CD3, CD8, CD44, OVA-tetramer, KLRG1, and CD127 (IL-7Ra) (PMID: 14625547). For mice that have cleared tumors by day 18, lymphocytes are isolated from the draining lymph node and the spleen by removing these organs and dissociating cells by smashing on a 70 um cell strainer (PMID: 14625547). Lymphocytes and splenocytes are then stained with antibody as described above.

TABLE 1
Treatment schedule.

| | | Treatment schedule | Treatment |
|---|---|---|---|
| Treatment | | anti-PD-1 (29f.1a12) | schedule |
| group | Cell line | or isotype (rIgG2a) | CYT107 |
| rIgG2a | MC38-OVA/ | Days 9, 12, 15 | no rx |
| | CT26-OVA | | |
| anti-PD-1 | MC38-OVA/ | Days 9, 12, 15 | no rx |
| | CT26-OVA | | |
| CYT107 + | MC38-OVA/ | Days 9, 12, 15 | Days 9, 12, 15 |
| rIgG2a | CT26-OVA | | |
| anti-PD-1 + | MC38-OVA/ | Days 9, 12, 15 | Days 9, 12, 15 |
| CYT107 | CT26-OVA | | |

Mice treated with either CYT107 alone or CYT107 and anti-PD-1 have an increase in memory precursor effector cell populations, as quantified by a relative increase in the percentage of cells falling into a KLRG1−CD127+ population, relative to mice treated with either isotype control or anti-PD-1 alone.

To demonstrate that this increase in memory CD8 T cell populations indeed results in greater protective immunity to cancer, mice are assayed for their ability to resist rechallenge with the same tumor cells after rejecting an established tumor. Mice are implanted with MC38 tumors, divided into three groups (˜20 mice per group) and treated with either CYT107 alone, anti-PD-1 alone, or CYT107 and anti-PD-1. Treatment for all mice begins on day 3 to ensure tumor rejection in the majority of animals. On day 45, mice that have cleared tumors are rechallenged with 1 million MC38 cells and then monitored for tumor growth every 3 days beginning on day 3 after tumor inoculation.

Mice cured of tumors by CYT107 + isotype or CYT107 + anti-PD-1 have a larger percentage of animals are also protected from rechallenge with MC38 cells than in mice treated with anti-PD-1 alone.

Human Experiments:

A fluorescent tetramer assay is a method for detecting antigen specific T cells. Tetramers (or dextramers) of MHC complexes bound by certain peptides are conjugated to fluorophores and used to stain T cells. T cells that stain positive with the peptide-MHC complexes have T cell receptor specificity for that antigen (PMID: 21690331). Tetramers of the melanocyte differentiation antigen, Melan-A, can be used to identify melanoma-specific T cells from populations of tumor-infiltrating leukocytes in HLA-A2+ patients (PMID: 10861093).

Patients with a diagnosis of measurable, unresectable, stage III or IV melanoma with a life expectancy of at least 4 months are randomized into groups receiving nivolumab alone or nivolumab plus CYT107 (rhlL-7). Nivolumab is administered every two weeks as in (PMID: 26406148) and CYT107 is administered at 20 μg/kg/week.

Prior to initiation of treatment, patients who are HLA-A2+ are identified using methods known to the art and biopsied before the onset of treatment and again two weeks after initiation of treatment. A third biopsy is taken 6 weeks after the onset of treatment during tumor regression to assay for antigen-specific CD8 T cells that are differentiating into memory cells. Biopsies are taken as described in (PMID: 20818844; PMID: 25428505). In short, biopsies are taken from cutaneous lesions and when possible, no individual lesion will be biopsied more than once. Leukocytes are isolated from biopsied tissue and immediately cryopreserved as described in (PMID: 21555851). In short, mononuclear cells are isolated by density gradient and then cryopreserved in RPMI 1640 with 40% FCS and 10% DMSO. Blood is collected from patients at the time of tumor biopsy and mononuclear cells are immediately isolated using a Ficoll gradient and then cryopreserved in RPMI 1640 with 40% FCS and 10% DMSO.

Leukocytes from both tumor biopsies and blood are directly stained for CD45, CD3, CD8, CD44, A2/Melan-A-tetramer, KLRG1, and CD127 (IL-7Ra) and analyzed on a BD Fortessa flow cytometer (PMID: 14625547).

Melan-A-specific CD8 T cells from both blood and tumor biopsies in patients receiving nivolumab and CYT107 contain a higher percentage of CD8 T cells falling into KLRG1−CD127+ and KLRG1+ CD127+ populations than into the KLRG1+ CD127− population. This difference is noted at both post-treatment biopsies, but may be more pronounced at the 6 week time point. In paired patient biopsies, an increase in the number of CD127+ cells is observed over time and the persistence of these cells is more pronounced in patients receiving CYT107 than patients receiving nivolumab alone.

### Example 6

### Combination Therapy for Stimulating Expansion of Thymocytes, Thymic Stroma, and Thymic Epithelium

To demonstrate the ability of the claimed invention to stimulate expansion of thymocytes, thymic stroma, and thymic epithelium, a clinical trial is conducted. Patients with a diagnosis of measurable, unresectable, stage III or IV melanoma with a life expectancy of at least 4 months are randomized into groups receiving nivolumab alone (the “control group”) or nivolumab plus palifermin (the “experimental” group). Patients in the experimental group are administered nivolumab every two weeks as described in (PMID: 26406148) as well as palifermin at a dose of 60 mcg/kg/day for three days prior to administration of the first dose of nivolumab, as well as a once weekly dose of 180 mcg/kg during weeks 1 through 6 for a total of 9 doses.

The primary endpoint is overall survival, defined as the time from randomization to the date of death. Secondary endpoints are progression-free survival, objective response rate and partial and complete response rates. Additional objectives of the trial include determining the association between palifermin therapy and thymus size, thymus cellularity, and thymus adiposity, and whether changes in thymus size, cellularity, or adiposity measures will correlate with clinical efficacy. In addition, several patients entering the trial agree to donate their thymuses in the event of their death for the purpose of studying the effects of palifermin on the thymus, including effects on thymocyte growth and expansion, as well as effects on thymic epithelium.

Patients from both the control group and the experimental group are subjected to MR and/or CT imaging during the course of therapy as well as in follow-up after the completion of the therapeutic regimen. During these imaging sessions, the thymus is imaged using mediastinal imaging methods and techniques known to the art (PMID: 21700977), and patients from the experimental group exhibit an enlarged thymus relative to control patients (PMID 20228326; 21628415) Furthermore, in at least some patients, enhancement of non-adipocyte tissues is apparent, as determined through reduced generalized T1-weighted signal, reduced fast spin-echo T2-weighted hyperintensity, diminishing intermediate T1- and T2-signal soft tissue, a combination thereof, or other imaging protocols known to the art to be capable of differentiating adipocytes from non-adipocytes. (PMID: 21700977)

Additionally, a subset of patients from both the experimental and control groups undergo a PET imaging study before and after the course of therapy described above. Uptake of the PET radiotracer FDG after therapy (compared to before therapy) will be higher in the experimental group than in the control group, thereby demonstrating the ability of the invention to boost thymic cellularity. (PMID: 11337547)

At the conclusion of the study, both primary and secondary endpoints are met; patients randomized to the experimental palifermin/nivolumab combination therapy exhibit a significant improvement in overall survival relative to patients in the control group randomized to nivolumab. Additionally, patients from the experimental combination therapy exhibit improved progression-free survival, overall survival, and exhibit a higher overall percentage of both partial and complete responses relative to patients in the control group.

After receiving the experimental therapy in the clinical trial described above, some patients may die. Upon physical examination during an autopsy of such a patient, the thymus is revealed to be significantly larger than that of age-matched pathological specimens from untreated deceased patients, and contain considerably more cells that are not adipocytes. This observation is confirmed when the excised thymus undergoes sectioning, fixation, and histological processing with hematoxylin and eosin staining, and microscopic examination to show that the thymus tissue has an expanded eosin-rich compartment comprising dense cellularity to age-matched controls.

The remainder of the autopsy-derived thymic tissue is processed as follows: Individual thymi are dissociated by smashing through a 70 um cell strainer and digesting in collagenase/dispase. Single cell thymic suspensions are enriched for thymic stroma and epithelium using Percoll density gradient centrifugation or directly stained for thymocyte populations (PMID: 24095736 ; PMID: 17988680; PMID: 25145384). A portion of the single-cell thymocyte suspension is used for direct staining with cell surface markers CD3, CD8, CD4, CD44, CD117, CD25, icTCRb, CDS; said cells are analyzed on BD Fortessa flow cytometer using methods known to art (PMID: 26485519). Single-cell stromal/epithelial enriched extracts from autopsy-derived experimental group thymuses are significantly enriched in thymic epithelial cells, thymic stromal cells, or a combination thereof relative to tissues derived from untreated controls, thereby demonstrating the ability of the therapeutic combination administered to the experimental group to stimulate expansion of thymocytes, thymic stroma, and thymic epithelium.

### Example 7

### IL-7 Production in Mammalian Cells

The DNA sequence encoding recombinant human IL-7, including an artificial signal peptide and a His tag (rhIL-7-His), was designed and synthesized, and the complete sequence was then subcloned into pcDNA3.1 vector (GenScript, Piscataway, N.J.) using the EcoRI and HindIII restriction enzymes. The cloned nucleotide sequence is listed here, where the IL-7 sequence is underlined, the artificial signal peptide is highlighted in grey, and the His tag is in italics:

SEQ ID NO: 17

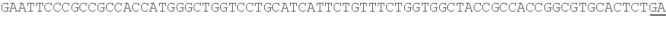

CTGTGATATCGAAGGCAAGGACGGCAAGCAGTACGAGTCCGTGCTGATGGTGTCCATCGACCAGCTGCTGGACTC
CATGAAGGAAATCGGCTCCAACTGCCTGAACAACGAGTTCAACTTCTTCAAGCGGCACATCTGCGACGCCAACAAA
GAAGGCATGTTCCTGTTCAGAGCCGCCAGAAAGCTGCGGCAGTTCCTGAAGATGAACTCCACCGGCGACTTCGAC
CTGCATCTGCTGAAAGTGTCTGAGGGCACCACCATCCTGCTGAACTGTACCGGCCAAGTGAAGGGCAGAAAGCCT
GCTGCTCTGGGCGAAGCCCAGCCTACCAAGTCTCTGGAAGAGAACAAGTCCCTGAAAGAGCAGAAGAAGCTGAA
CGACCTCTGCTTCCTGAAGCGGCTGCTGCAAGAGATCAAGACCTGCTGGAACAAGATCCTGATGGGCACCAAAGA
GCACCATCACCATCATCACCACTGATAAGCTT
SEQ ID NO: 18
GACTGTGATATCGAAGGCAAGGACGGCAAGCAGTACGAGTCCGTGCTGATGGTGTCCATCGACCAGCTGCTGGA
CTCCATGAAGGAAATCGGCTCCAACTGCCTGAACAACGAGTTCAACTTCTTCAAGCGGCACATCTGCGACGCCAAC
AAAGAAGGCATGTTCCTGTTCAGAGCCGCCAGAAAGCTGCGGCAGTTCCTGAAGATGAACTCCACCGGCGACTTC
GACCTGCATCTGCTGAAAGTGTCTGAGGGCACCACCATCCTGCTGAACTGTACCGGCCAAGTGAAGGGCAGAAAG
CCTGCTGCTCTGGGCGAAGCCCAGCCTACCAAGTCTCTGGAAGAGAACAAGTCCCTGAAAGAGCAGAAGAAGCTG
AACGACCTCTGCTTCCTGAAGCGGCTGCTGCAAGAGATCAAGACCTGCTGGAACAAGATCCTGATGGGCACCAAA
GAGCAC
SEQ ID NO: 19
ATGGGCTGGTCCTGCATCATTCTGTTTCTGGTGGCTACCGCCACCGGCGTGCACTCT
SEQ ID NO: 20
CATCACCATCATCACCAC
SEQ ID NO: 21

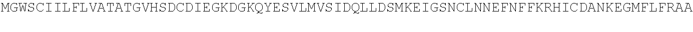

RKLRQFLKMNSTGDFDLHLLKVSEGTTILLNCTGQVKGRKPAALGEAQPTKSLEENSKLKEQKKLNDLCFLKRLLQEIKTC

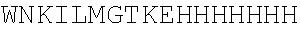

The vector containing IL-7 was transfected into CHO-3E7 cells which were grown in serum-free FreeStyle CHO Expression Medium (Life Technologies, Carlsbad, Calif., USA). The cells were maintained in Erlenmeyer Flasks (Corning Inc., Acton, Mass.) at 37° C. with 5% CO₂ on an orbital shaker (VWR Scientific, Chester, Pa.). One day prior to transfection, the cells were seeded at 3.5×10⁶ cells per mL in Corning Erlenmeyer Flasks. On the day of transfection, DNA and transfection reagent were mixed at an optimal ratio and then added into the flask with cells ready for transfection.

The recombinant plasmid encoding target protein was transiently transfected into 100 ml suspension CHO-3E7 cell cultures. The cell culture supernatants collected on day 2, 4 and 5 post-transfection were used for the protein expression evaluation. The cell culture supernatant harvested on day 6 was used for purification.

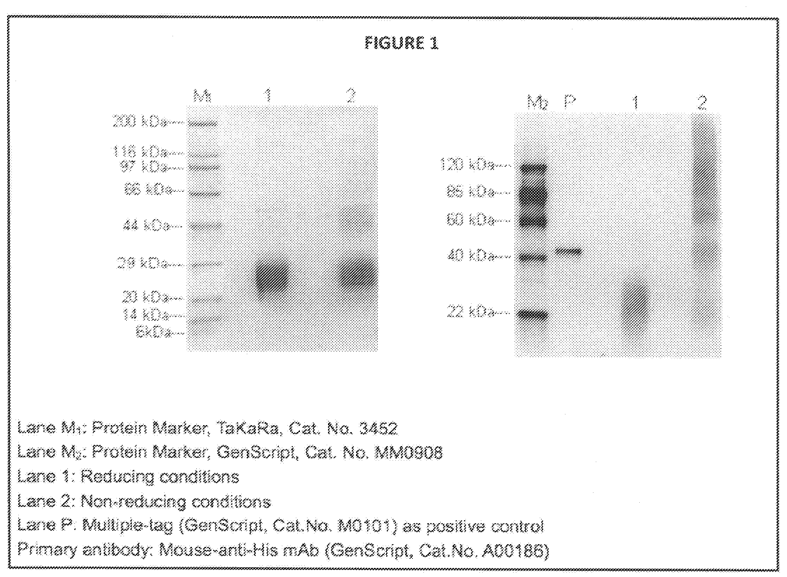

To estimate the protein expression levels, cell culture supernatants collected on day 2, 4 and 5 post-transfection were analyzed by SDS-PAGE and western blot. For protein purification, cell culture supernatant was harvested on day 6 post-transfection. 0.5 ml Ni Sepharose 6 Fast Flow (GE, Lot 10173458) was added to cell culture supernatant, incubated for 3˜4 hours to capture the target protein, followed by washing and elution with buffer containing 20 mM sodium phosphate, 0.5 M NaCl, 500 mM imidazole, pH 7.4. The eluted fractions were analyzed by SDS-PAGE and western blot. The primary antibody used for western blots was mouse-anti-his mAb (GenScript, Cat. A00186). FIG. 1 shows the resulting SDS-PAGE image on the left and western blot data on the right.

### Example 8

### IL-7-Fc-Receptor Fusion Production in Mammalian Cells

The DNA sequence encoding recombinant human IL-7-Fc-receptor fusion (rhIL-7-Fc), including an artificial signal peptide, was designed and synthesized, and the complete sequence was then subcloned into pcDNA3.1 vector (GenScript, Piscataway, N.J.) for CHO-3E7 cell expression using the EcoRI and HindIII restriction enzymes. The cloned nucleotide sequence is listed here, where the sequence encoding rhIL-7-Fc is underlined and the artificial signal peptide is highlighted in grey:

SEQ ID NO: 22

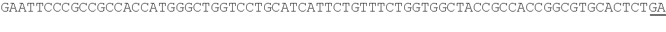

CTGTGATATCGAAGGCAAGGACGGCAAGCAGTACGAGTCCGTGCTGATGGTGTCCATCGACCAGCTGCTGGACTC
CATGAAGGAAATCGGCTCCAACTGCCTGAACAACGAGTTCAACTTCTTCAAGCGGCACATCTGCGACGCCAACAAA
GAAGGCATGTTCCTGTTCAGAGCCGCCAGAAAGCTGCGGCAGTTCCTGAAGATGAACTCCACCGGCGACTTCGAC
CTGCATCTGCTGAAAGTGTCTGAGGGCACCACCATCCTGCTGAACTGTACCGGCCAAGTGAAGGGCAGAAAGCCT
GCTGCTCTGGGCGAAGCCCAGCCTACCAAGTCTCTGGAAGAGAACAAGTCCCTGAAAGAGCAGAAGAAGCTGAA
CGACCTCTGCTTCCTGAAGCGGCTGCTGCAAGAGATCAAGACCTGCTGGAACAAGATCCTGATGGGCACCAAAGA
GCATGGCGGCGGAGGATCTGGCGGAGGTGGAAGCGGAGGCGGTGGATCTGAACCTAAGTCCTGCGACAAGACC
CACACCTGTCCTCCATGTCCTGCTCCAGAACTGCTCGGCGGACCTTCCGTGTTTCTGTTCCCTCCAAAGCCTAAGGA
CACCCTGATGATCTCTCGGACCCCTGAAGTGACCTGCGTGGTGGTGGATGTGTCTCACGAGGATCCCGAAGTGAA
GTTCAATTGGTACGTGGACGGCGTGGAAGTGCACAACGCCAAGACCAAGCCTAGAGAGGAACAGTACAACTCCA
CCTACAGAGTGGTGTCTGTGCTGACCGTGCTGCACCAGGATTGGCTGAACGGCAAAGAGTACAAGTGCAAGGTGT
CCAACAAGGCCCTGCCTGCTCCTATCGAAAAGACCATCTCCAAGGCCAAGGGCCAGCCTAGGGAACCCCAGGTTT
ACACCTTGCCTCCATCTCGGGACGAGCTGACCAAGAACCAGGTGTCCCTGACCTGTCTGGTCAAGGGCTTCTACCC
CTCCGATATCGCCGTGGAATGGGAGTCTAATGGCCAGCCTGAGAACAACTACAAGACAACCCCTCCTGTGCTGGA
CAGCGACGGCTCATTCTTCCTGTACTCCAAGCTGACAGTGGACAAGTCCAGATGGCAGCAGGGCAACGTGTTCTCC
TGCTCCGTGATGCACGAGGCCCTGCACAATCACTACACCCAGAAGTCCCTGTCTCTGTCCCCTGGCAAGTGATAAG
CTT
SEQ ID NO: 23
GACTGTGATATCGAAGGCAAGGACGGCAAGCAGTACGAGTCCGTGCTGATGGTGTCCATCGACCAGCTGCTGGA
CTCCATGAAGGAAATCGGCTCCAACTGCCTGAACAACGAGTTCAACTTCTTCAAGCGGCACATCTGCGACGCCAAC
AAAGAAGGCATGTTCCTGTTCAGAGCCGCCAGAAAGCTGCGGCAGTTCCTGAAGATGAACTCCACCGGCGACTTC
GACCTGCATCTGCTGAAAGTGTCTGAGGGCACCACCATCCTGCTGAACTGTACCGGCCAAGTGAAGGGCAGAAAG
CCTGCTGCTCTGGGCGAAGCCCAGCCTACCAAGTCTCTGGAAGAGAACAAGTCCCTGAAAGAGCAGAAGAAGCTG
AACGACCTCTGCTTCCTGAAGCGGCTGCTGCAAGAGATCAAGACCTGCTGGAACAAGATCCTGATGGGCACCAAA
GAGCATGGCGGCGGAGGATCTGGCGGAGGTGGAAGCGGAGGCGGTGGATCTGAACCTAAGTCCTGCGACAAGA
CCCACACCTGTCCTCCATGTCCTGCTCCAGAACTGCTCGGCGGACCTTCCGTGTTTCTGTTCCCTCCAAAGCCTAAG
GACACCCTGATGATCTCTCGGACCCCTGAAGTGACCTGCGTGGTGGTGGATGTGTCTCACGAGGATCCCGAAGTG
AAGTTCAATTGGTACGTGGACGGCGTGGAAGTGCACAACGCCAAGACCAAGCCTAGAGAGGAACAGTACAACTC
CACCTACAGAGTGGTGTCTGTGCTGACCGTGCTGCACCAGGATTGGCTGAACGGCAAAGAGTACAAGTGCAAGGT
GTCCAACAAGGCCCTGCCTGCTCCTATCGAAAAGACCATCTCCAAGGCCAAGGGCCAGCCTAGGGAACCCCAGGT
TTACACCTTGCCTCCATCTCGGGACGAGCTGACCAAGAACCAGGTGTCCCTGACCTGTCTGGTCAAGGGCTTCTAC
CCCTCCGATATCGCCGTGGAATGGGAGTCTAATGGCCAGCCTGAGAACAACTACAAGACAACCCCTCCTGTGCTG
GACAGCGACGGCTCATTCTTCCTGTACTCCAAGCTGACAGTGGACAAGTCCAGATGGCAGCAGGGCAACGTGTTC
TCCTGCTCCGTGATGCACGAGGCCCTGCACAATCACTACACCCAGAAGTCCCTGTCTCTGTCCCCTGGCAAG
SEQ ID NO: 19
ATGGGCTGGTCCTGCATCATTCTGTTTCTGGTGGCTACCGCCACCGGCGTGCACTCT
SEQ ID NO: 24

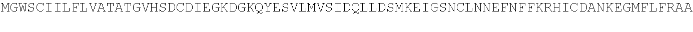

RKLRQFLKMNSTGDFDLHLLKVSEGTTILLNCTGQVKGRKPAALGEAQPTKSLEENKSLKEQKKLNDLCFLKRLLQEIKTC

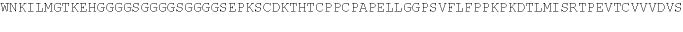

HEDPEVKFNWYVDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSKLTVDKSRWQQGNVFS
CSVMHEALHNHYTQKSLSLSPGK

The vector containing rhIL-7-Fc was transfected into CHO-3E7 cells, which were grown in serum-free FreeStyle CHO Expression Medium (Life Technologies, Carlsbad, Calif., USA). The cells were maintained in erlenmeyer flasks (Corning Inc., Acton, Mass.) at 37° C. with 5% CO₂ on an orbital shaker (VWR Scientific, Chester, Pa.). One day prior to transfection, the cells were seeded at 3.5×10⁶ cells per mL in Corning erlenmeyer flasks. On the day of transfection, DNA and transfection reagent were mixed at an optimal ratio and then added into the flask with cells ready for transfection.

The recombinant plasmid encoding target protein was transiently transfected into 100 ml suspension CHO-3E7 cell cultures. The cell culture supernatants collected on day 2, 4 and 5 post-transfection were used for the protein expression evaluation. The cell culture supernatant harvested on day 6 was used for purification.

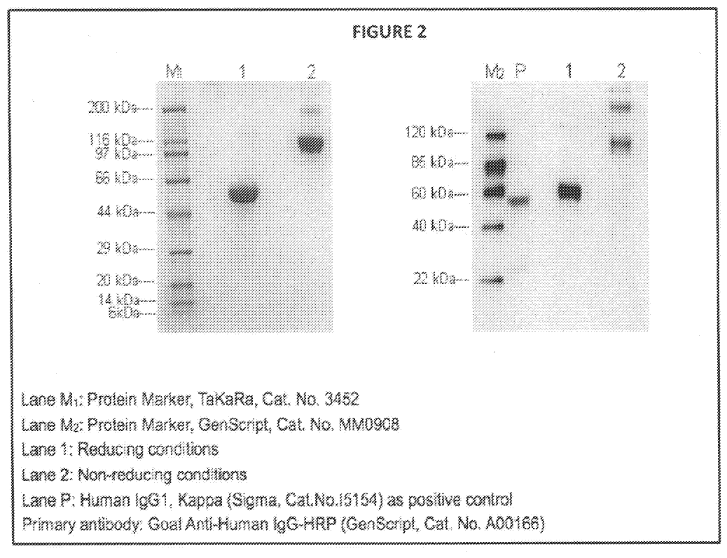

Cell culture broth was centrifuged and followed by filtration to remove any remaining cells. Filtered cell culture supernatant was loaded onto a protein-A purification column at an appropriate flowrate. After washing and elution with 100 mM citrate, pH 3.0, the eluted fractions were pooled and buffer exchanged to PBS buffer at pH 7.2. The purified protein was analyzed by SDS-PAGE, western blotting and SEC-HPLC analysis for molecular weight and purity measurements. The concentration was determined by Bradford assay with BSA as a standard. 360 μg of protein was produced at >90% purity. FIG. 2 shows the resulting SDS-PAGE image on the left and western blot data on the right.

### Example 9

### Combination Therapy of IL-7 and Anti-PD-L1 to Treat Mouse Colorectal Cancer

The primary purpose of the study was to determine the anti-tumor activity of IL-7 in combination with anti-mPD-L1 antibody against established CT26.WT murine colorectal tumors in female Balb/c mice. Recombinant human IL-7 containing a C-terminal His-6 tag (rhIL-7-His), as described in example 7, was prepared as a 1 mg/ml stock solution, with purity >95%, in sterile water and 1% bovine serum albumin (BSA). The dosing solution was prepared by diluting the stock solution with PBS to 0.16mg/ml and had a pH value of 7.4. Anti-mPD-L1 antibody (clone: 10F.9G2, 4.68 mg/ml, Lot 598816M1) was obtained from BioXCell and was prepared by diluting the stock solution with PBS to 0.4 mg/mL and had a pH value of 7.3. Rat IgG2b (clone: LTF-2, 8.68 mg/ml, Lot 5535-4-6/0815) was obtained from BioXCell to serve as the isotype control and was prepared in the same manner as anti-mPD-L1 antibody.

Female Balb/c mice (BALB/cAnNHsd) that were 8-9 weeks old were used in the study. The animals were fed irradiated Harlan 2918.15 rodent diet and water ad libitum. CT26.WT cells were obtained from ATCC. They were grown in RPMI 1640 medium which was modified with 1% 100 mM Na pyruvate, 1% 1M HEPES buffer, 1% of a 45% glucose solution and supplemented with 10% non-heat-inactivated fetal bovine serum (FBS) and 1% 100× penicillin/streptomycin/L-glutamine (PSG). The growth environment was maintained in an incubator with a 5% CO₂ atmosphere at 37° C. When expansion was complete, the cells (passage 5) were trypsinized using 0.25% trypsin-EDTA solution. Following cell detachment, the trypsin was inactivated by dilution with complete growth medium and any clumps of cells were separated by pipetting. The cells were centrifuged at 200 rcf for 8 minutes at 4° C., the supernatant was aspirated, and the pellet was re-suspended in cold Dulbecco's phosphate buffered saline (DPBS) by pipetting. An aliquot of the homogeneous cell suspension was diluted in a trypan blue solution and counted using a Luna automated cell counter. The pre-implantation cell viability was 93%. The cell suspension was centrifuged at 200 rcf for 8 minutes at 4° C. The supernatant was aspirated and the cell pellet was re-suspended in cold serum-free medium to generate a final concentration of 2.50E+06 trypan-excluding cells/ml. The cell suspension was maintained on wet ice during implantation. Following implantation, an aliquot of the remaining cells was diluted with a trypan blue solution and counted to determine the post-implantation cell viability (97%). 3 of 3 thioglycolate cultures of donor tumor cells used for implantation of this study were negative for gross bacterial contamination.

Test animals were implanted subcutaneously, high in the axilla (just under the fore limb) on Day 0 with 5.0E+05 cells in 0.2 ml of serum-free medium using a 27-gauge needle and syringe.

All mice were sorted into study groups, 10 mice each, based on caliper measurement estimation of tumor burden. The mice were distributed to ensure that the mean tumor burden for all groups was within 10% of the overall mean tumor burden for the study population. Treatment began on day 10 at an overall mean tumor burden of 138mm³ (range of group means, 133-141mm³). All mice were dosed with a fixed volume of 0.5 ml on the day of treatment.
Group 1: Isotype control, 0.2 mg/injection, intraperitoneal (IP)
Group 2: anti-mPD-L1 Ab, 0.2 mg/injection, IP
Group 3: rhIL-7-His, 0.02 mg/injection, IP
Group 4: anti-mPD-L1 Ab+rhIL-7-His, 0.2mg/injection+0.08mg/injection, IP+IP
Group 5: anti-mPD-L1 Ab+rhIL-7-His, 0.2mg/injection+0.02mg/injection, IP+IP

Tumor measurements were recorded three times weekly. Tumor burden (mm³) was estimated from caliper measurements by the formula for the volume of a prolate ellipsoid assuming unit density as: Tumor burden (mm³)=(L×W)²/₂, where L and W are the respective orthogonal tumor length and width measurements (mm).

The primary endpoints used to evaluate efficacy were: tumor growth delay, median T/ C, complete and partial tumor response, and the number of tumor-free survivors at the end of the study. Tumor growth delay for this experiment was evaluated at a selected evaluation size of 1000mm³. In this experiment, median T/C was evaluated on day 24. Day 24 was the last day the median animal was still alive in the isotype control group. A complete response (CR) is defined as a decrease in tumor mass to an undetectable size (<50 mm³), and a partial response (PR) is defined as a 50% decrease in tumor mass from that at first treatment. PRs are exclusive of CRs, as are tumor-free survivors (TFS).

The mean estimated tumor burden for all groups in the experiment on the first day of treatment was 138 mm³ and all of the groups in the experiment were well-matched (range of group means, 133-141 mm³). All animals weighed at least 16.0 g at the initiation of therapy. Mean group body weights at first treatment were also well-matched (range, 18.3-19.4 g). A tumor burden of 1000 mm³ was chosen for evaluation of efficacy by tumor growth delay. The median isotype control tumor reached 1000 mm³ on day 20.7, and the tumor volume doubling time for the isotype control group was 3.5 days (historically 2-4 days).

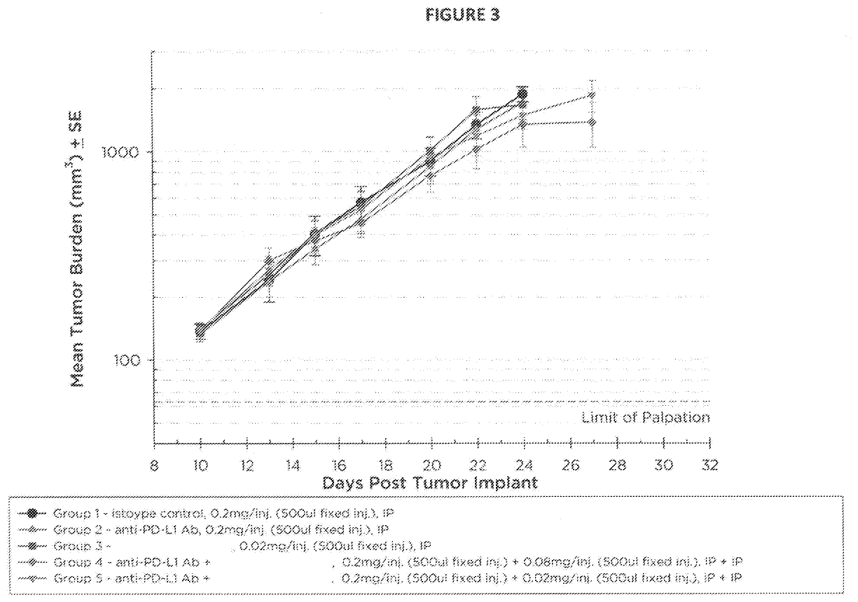

Treatment with anti-mPD-L1 antibody at 0.2 mg/injection failed to produce activity based on the group endpoints of tumor growth delay. Treatment with rhIL-7-His as a monotherapy at 0.02mg/injection was ineffective, producing negligible tumor growth delays. Treatment with rhIL-7-His in combination with anti-PD-L1 antibody at 0.08 mg/injection failed to produce activity based on the group endpoints of tumor growth delay. A similar combination regimen with rhIL-7-His at 0.02 mg/inj was ineffective. FIG. 3 shows the mean tumor burden in each treatment group.

### Example 10

### Analysis of IL7 and IL-7 Receptor Expression in Human Tumors

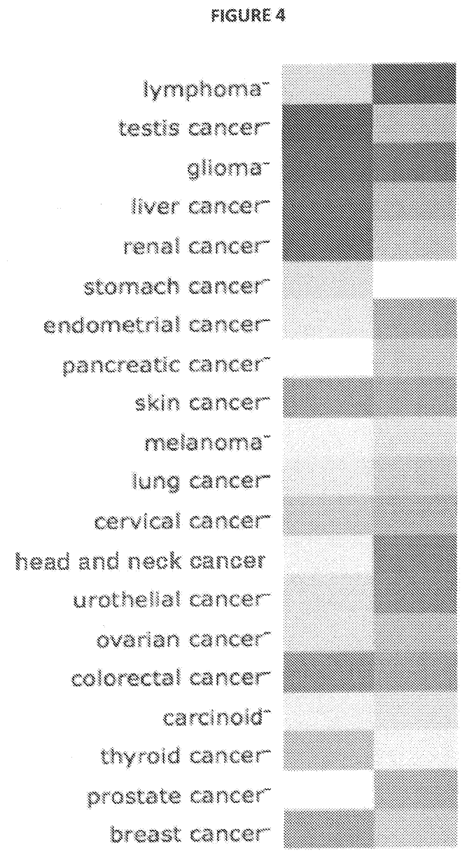

This example describes the examination of human tumor samples for protein levels of IL-7 and IL-7 receptor. The human protein atlas database was examined to identify the expression level of IL-7 and IL-7 receptor in a range of human cancers (PubMed: 25613900). The database contains gene expression data based on protein expression patterns in 216 different cancer samples representing the 20 most common forms of human cancer. Protein expression data is derived from antibody-based protein profiling using immunohistochemistry. As shown in FIG. 4, the relative expression level of IL7 and IL-7R showed significant variation in different cancer types. Notably, cancers including colorectal cancer and breast cancer had high relative IL-7 and IL-7 receptor expression while cancers including melanoma, pancreatic cancer, and liver cancer expressed relatively lower levels of IL-7 and IL-7 receptor. This unexpected variability suggests that screening a cancer for expression of IL-7 and/or ll-7R is useful in determining an appropriate therapy as described herein.

SEQUENCE LISTING:
Human IL-7 protein sequence, or isoform 1,
SEQ ID NO: 1
DCDIEGKDGKQYESVLMVSIDQLLDSMKEIGSNCLNNEFNFFKRHICDANKEGMFLFRAARKLRQFLKMNSTGDFDLH
LLKVSEGTTILLNCTGQVKGRKPAALGEAQPTKSLEENKSLKEQKKLNDLCFLKRLLQEIKTCWNKILMGTKEH
Human IL-7 protein sequence, or isoform 2,
SEQ ID NO: 2
DCDIEGKDGKQYESVLMVSIDQLLDSMKEIGSNCLNNEFNFFKRHICDANKVKGRKPAALGEAQPTKSLEENKSLKEQK
KLNDLCFLKRLLQEIKTCWNKILMGTKEH
Human IL-7 protein sequence, or isoform 3,
SEQ ID NO: 3
DCDIEGKDGKQYESVLMVSIDQLLDSMKEIGSNCLNNEFNFFKRHICDANKEGMFLFRAARKLRQFLKMNSTGDFDLH
LLKVSEGTTILLNCTGQEENKSLKEQKKLNDLCFLKRLLQEIKTCWNKILMGTKEH
Human IL-7 protein sequence, or isoform 4,
SEQ ID NO: 4
DCDIEGKDGKQYESVLMVSIDQLLDSMKEIGSNCLNNEFNFFKRHICDANKEENKSLKEQKKLNDLCFLKRLLQEIKTCW
NKILMGTKEH
Human IL-7 protein sequence, or delta 5 (Vudattu),
SEQ ID NO: 5
DCDIEGKDGKQYESVLMVSIDQLLDSMKEIGSNCLNNEFNFFKRHICDANKEGMFLFRAARKLRQFLKMNSTGDFDLH
LLKVSEGTTILLKEQKKLNDLCFLKRLLQEIKTCWNKILMGTKEH
Human IL-7 protein sequence, or delta 5,
SEQ ID NO: 6
DCDIEGKDGKQYESVLMVSIDQLLDSMKEIGSNCLNNEFNFFKRHICDANKEGMFLFRAARKLRQFLKMNSTGDFDLH
LLKVSEGTTILLNCTGQEENKSLKEQKKLNDLCFLKRLLQEIKTCWNKILMGTKEH
Human IL-7 protein sequence, or delta 5/6,
SEQ ID NO: 7
DCDIEGKDGKQYESVLMVSIDQLLDSMKEIGSNCLNNEFNFFKRHICDANKEGMFLFRAARKLRQFLKMNSTGDFDLH
LLKVSEGTTILLNCTGQH
Human IL-7 protein sequence, or delta 4,
SEQ ID NO: 8
DCDIEGKDGKQYESVLMVSIDQLLDSMKEIGSNCLNNEFNFFKRHICDANKVKGRKPAALGEAPTKSLEENKSLKEQK
KLNDLCFLKRLLQEIKTCWNKILMGTKEH
Human IL-7 protein sequence, or delta 4 (C-S),
SEQ ID NO: 9
DCDIEGKDGKQYESVLMVSIDQLLDSMKEIGSNCLNNEFNFFKRHISDANKVKGRKPAALGEAQPTKSLEENKSLKEQK
KLNDLCFLKRLLQEIKTCWNKILMGTKEH
Human IL-7 protein sequence, or delta 2,
SEQ ID NO: 10
MKEIGSNCLNNEFNFFKRHICDANKEGMFLFRAARKLRQFLKMNSTGDFDLHLLKVSEGTTILLNCTGQVKGRKPAALG
EAQPTKSLEENKSLKEQKKLNDLCFLKRLLQEIKTCWNKILMGTKEH
Human IL-7 HGF protein sequence,
SEQ ID NO: 11
DCDIEGKDGKQYESVLMVSIDQLLDSMKEIGSNCLNNEFNFFKRHICDANKEGMFLFRAARKLRQFLKMNSTGDFDLH
LLKVSEGTTILLNCTGQVKGRKPAALGEAQPTKSLEENKSLKEQKKLNDLCFLKRLLQEIKTCWNKILMGTKEGGGGSGG
GGSGGGGSVVNGIPTRTNIGWMVSLRYRNKHICGGSLIKESWVLTARQCFPSRDLKDYEAWLGIHDVHGRGDEKCKQ
VLNVSQLVYGPEGSDLVLMKLARPAVLDDFVSTIDLPNYGCTIPEKTSCSVYGWGYTGLINYDGLLRVAHLYIMGNEKCS
QHHRGKVTLNESEICAGAEKIGSGPCEGDYGGPLVCEQHKMRMVLGVIVPGRGCAIPNRPGIFVRVAYYAKWIHKIILT
YKVPQS
Human IL-7-IL-15 protein sequence,
SEQ ID NO: 12
DCDIEGKDGKQYESVLMVSIDQLLDSMKEIGSNCLNNEFNFFKRHICDANKEGMFLFRAARKLRQFLKMNSTGDFDLH
LLKVSEGTTILLNCTGQVKGRKPAALGEAQPTKSLEENKSLKEQKKLNDLCFLKRLLQEIKTCWNKILMGTKEHGGGGSG
GGGSGGGGSMNWVNVISDLKKIEDLIQSMHIDATLYTESDVHPSCKVTAMKCFLLELQVISLESGDASIHDTVENLIILA
NNSLSSNGNVTESGCKECEELEEKNIKEFLQSFVHIVQMFINTS
Human IL-7-Fc protein sequence,
SEQ ID NO: 13
DCDIEGKDGKQYESVLMVSIDQLLDSMKEIGSNCLNNEFNFFKRHICDANKEGMFLFRAARKLRQFLKMNSTGDFDLH
LLKVSEGTTILLNCTGQVKGRKPAALGEAQPTKSLEENKSLKEQKKLNDLCFLKRLLQEIKTCWNKILMGTKEHGGGGSG
GGGSGGGGSEPKSCDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLYVDKSRWQQGNVFSCSVMHEALHNHYTQKSL
SLSPGK
Human precursor peptide sequence,
SEQ ID NO: 14
MFHVSFRYIFGLPPLILVLLPVASS
Human leader peptide sequence,
SEQ ID NO: 15
MGWSCIILFLVATATGVHS
HIS tag peptide sequence,
SEQ ID NO: 16
HHHHHH
Construct with human IL-7 nucleotide sequence,
SEQ ID NO: 17

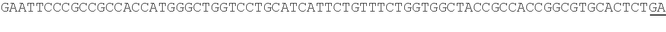

CTGTGATATCGAAGGCAAGGACGGCAAGCAGTACGAGTCCGTGCTGATGGTGTCCATCGACCAGCTGCTGGACTC
CATGAAGGAAATCGGCTCCAACTGCCTGAACAACGAGTTCAACTTCTTCAAGCGGCACATCTGCGACGCCAACAAA
GAAGGCATGTTCCTGTTCAGAGCCGCCAGAAAGCTGCGGCAGTTCCTGAAGATGAACTCCACCGGCGACTTCGAC
CTGCATCTGCTGAAAGTGTCTGAGGGCACCACCATCCTGCTGAACTGTACCGGCCAAGTGAAGGGCAGAAAGCCT
GCTGCTCTGGGCGAAGCCCAGCCTACCAAGTCTCTGGAAGAGAACAAGTCCCTGAAAGAGCAGAAGAAGCTGAA
CGACCTCTGCTTCCTGAAGCGGCTGCTGCAAGAGATCAAGACCTGCTGGAACAAGATCCTGATGGGCACCAAAGA
GCACCATCACCATCATCACCACTGATAAGCTT
Human IL-7 nucleotide sequence,
SEQ ID NO: 18
GACTGTGATATCGAAGGCAAGGACGGCAAGCAGTACGAGTCCGTGCTGATGGTGTCCATCGACCAGCTGCTGGA
CTCCATGAAGGAAATCGGCTCCAACTGCCTGAACAACGAGTTCAACTTCTTCAAGCGGCACATCTGCGACGCCAAC
AAAGAAGGCATGTTCCTGTTCAGAGCCGCCAGAAAGCTGCGGCAGTTCCTGAAGATGAACTCCACCGGCGACTTC
GACCTGCATCTGCTGAAAGTGTCTGAGGGCACCACCATCCTGCTGAACTGTACCGGCCAAGTGAAGGGCAGAAAG
CCTGCTGCTCTGGGCGAAGCCCAGCCTACCAAGTCTCTGGAAGAGAACAAGTCCCTGAAAGAGCAGAAGAAGCTG
AACGACCTCTGCTTCCTGAAGCGGCTGCTGCAAGAGATCAAGACCTGCTGGAACAAGATCCTGATGGGCACCAAA
GAGCAC
Artificial signal peptide nucleotide sequence,
SEQ ID NO: 19
ATGGGCTGGTCCTGCATCATTCTGTTTCTGGTGGCTACCGCCACCGGCGTGCACTCT
HIS tag nucleotide sequence,
SEQ ID NO: 20
CATCACCATCATCACCAC
Construct with human IL-7 protein sequence,
SEQ ID NO: 21

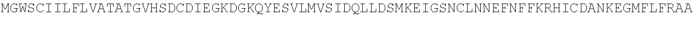

RKLRQFLKMNSTGDFDLHLLKVSEGTTILLNCTGQVKGRKPAALGEAQPTKSLEENKSLKEQKKLNDLCFLKRLLQEIKTC

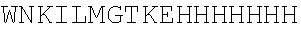

Construct with human IL-7-Fc nucleotide sequence,
SEQ ID NO: 22

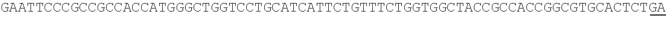

CTGTGATATCGAAGGCAAGGACGGCAAGCAGTACGAGTCCGTGCTGATGGTGTCCATCGACCAGCTGCTGGACTC
CATGAAGGAAATCGGCTCCAACTGCCTGAACAACGAGTTCAACTTCTTCAAGCGGCACATCTGCGACGCCAACAAA
GAAGGCATGTTCCTGTTCAGAGCCGCCAGAAAGCTGCGGCAGTTCCTGAAGATGAACTCCACCGGCGACTTCGAC
CTGCATCTGCTGAAAGTGTCTGAGGGCACCACCATCCTGCTGAACTGTACCGGCCAAGTGAAGGGCAGAAAGCCT
GCTGCTCTGGGCGAAGCCCAGCCTACCAAGTCTCTGGAAGAGAACAAGTCCTGAAAGAGCAGAAGAAGCTGAA
CGACCTCTGCTTCCTGAAGCGGCTGCTGCAAGAGATCAAGACCTGCTGGAACAAGATCCTGATGGGCACCAAAGA
GCATGGCGGCGGAGGATCTGGCGGAGGTGGAAGCGGAGGCGGTGGATCTGAACCTAAGTCCTGCGACAAGACC
CACACCTGTCCTCCATGTCCTGCTCCAGAACTGCTCGGCGGACCTTCCGTGTTTCTGTTCCCTCCAAAGCCTAAGGA
CACCCTGATGATCTCTCGGACCCCTGAAGTGACCTGCGTGGTGGTGGATGTGTCTCACGAGGATCCCGAAGTGAA
GTTCAATTGGTACGTGGACGGCGTGGAAGTGCACAACGCCAAGACCAAGCCTAGAGAGGAACAGTACAACTCCA
CCTACAGAGTGGTGTCTGTGCTGACCGTGCTGCACCAGGATTGGCTGAACGGCAAAGAGTACAAGTGCAAGGTGT
CCAACAAGGCCCTGCCTGCTCCTATCGAAAAGACCATCTCCAAGGCCAAGGGCCAGCCTAGGGAACCCCAGGTTT
ACACCTTGCCTCCATCTCGGGACGAGCTGACCAAGAACCAGGTGTCCCTGACCTGTCTGGTCAAGGGCTTCTACCC
CTCCGATATCGCCGTGGAATGGGAGTCTAATGGCCAGCCTGAGAACAACTACAAGACAACCCCTCCTGTGCTGGA
CAGCGACGGCTCATTCTTCCTGTACTCCAAGCTGACAGTGGACAAGTCCAGATGGCAGCAGGGCAACGTGTTCTCC
TGCTCCGTGATGCACGAGGCCCTGCACAATCACTACACCCAGAAGTCCCTGTCTCTGTCCCCTGGCAAGTGATAAG
CTT
Human IL-7-Fc nucleotide sequence,
SEQ ID NO: 23
GACTGTGATATCGAAGGCAAGGACGGCAAGCAGTACGAGTCCGTGCTGATGGTGTCCATCGACCAGCTGCTGGA
CTCCATGAAGGAAATCGGCTCCAACTGCCTGAACAACGAGTTCAACTTCTTCAAGCGGCACATCTGCGACGCCAAC
AAAGAAGGCATGTTCCTGTTCAGAGCCGCCAGAAAGCTGCGGCAGTTCCTGAAGATGAACTCCACCGGCGACTTC
GACCTGCATCTGCTGAAAGTGTCTGAGGGCACCACCATCCTGCTGAACTGTACCGGCCAAGTGAAGGGCAGAAAG
CCTGCTGCTCTGGGCGAAGCCCAGCCTACCAAGTCTCTGGAAGAGAACAAGTCCCTGAAAGAGCAGAAGAAGCTG
AACGACCTCTGCTTCCTGAAGCGGCTGCTGCAAGAGATCAAGACCTGCTGGAACAAGATCCTGATGGGCACCAAA
GAGCATGGCGGCGGAGGATCTGGCGGAGGTGGAAGCGGAGGCGGTGGATCTGAACCTAAGTCCTGCGACAAGA
CCCACACCTGTCCTCCATGTCCTGCTCCAGAACTGCTCGGCGGACCTTCCGTGTTTCTGTTCCCTCCAAAGCCTAAG
GACACCCTGATGATCTCTCGGACCCCTGAAGTGACCTGCGTGGTGGTGGATGTGTCTCACGAGGATCCCGAAGTG
AAGTTCAATTGGTACGTGGACGGCGTGGAAGTGCACAACGCCAAGACCAAGCCTAGAGAGGAACAGTACAACTC
CACCTACAGAGTGGTGTCTGTGCTGACCGTGCTGCACCAGGATTGGCTGAACGGCAAAGAGTACAAGTGCAAGGT
GTCCAACAAGGCCCTGCCTGCTCCTATCGAAAAGACCATCTCCAAGGCCAAGGGCCAGCCTAGGGAACCCCAGGT
TTACACCTTGCCTCCATCTCGGGACGAGCTGACCAAGAACCAGGTGTCCCTGACCTGTCTGGTCAAGGGCTTCTAC
CCCTCCGATATCGCCGTGGAATGGGAGTCTAATGGCCAGCCTGAGAACAACTACAAGACAACCCCTCCTGTGCTG
GACAGCGACGGCTCATTCTTCCTGTACTCCAAGCTGACAGTGGACAAGTCCAGATGGCAGCAGGGCAACGTGTTC
TCCTGCTCCGTGATGCACGAGGCCCTGCACAATCACTACACCCAGAAGTCCCTGTCTCTGTCCCCTGGCAAG
Construct with human IL-7-Fc protein sequence,
SEQ ID NO: 24

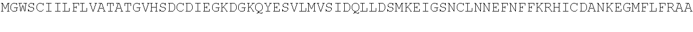

RKLRQFLKMNSTGDFDLHLLKVSEGTTILLNCTGQVKGRKPAALGEAQPTKSLEENKSLKEQKKLNDLCFLKRLLQEIKTC

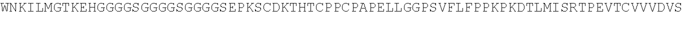

HEDPEVKFVWYVDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSKLTVDKSRWQQGNVFS
CSVMHEALHNHYTQKSLSLSPGK

1.-20. (canceled)

21. A method of decreasing T-cell exhaustion in a subject in need thereof, comprising:
administering to the subject an agent that modulates thymus or T cell function in combination with an inhibitor of immune checkpoint, wherein the subject has an expression of a cytokine receptor chain in a Tumor-infiltrating Lymphocyte (TIL) at least 10% higher than that of a non-memory effector cell from a healthy control, wherein the cytokine receptor chain is CD127, CD25, CD122, CD124, CD360 or CD132.

22. The method of claim 21, further comprising, prior to administering, assessing one or more markers for T cell exhaustion in the subject.

23. The method of claim 22, wherein the one or more markers comprise one or more transcription factors selected from: eomesodermin, T-bet, GATA-3, BCL-6, Helios, NFAT, Blimp-1, FoxO1, c-myc, or a combination thereof.

24. The method of claim 22, wherein the one or more markers comprise one or more cytokines selected from: IL-7, IL-2, IL-4, IL-9, IL-15, IL-21, and a combination thereof, and wherein an expression level of the one or more cytokines of a tumor biopsy from the subject is lower than that of a biopsy from a healthy tissue.

25. The method of claim 21, wherein, after administering, the subject has (1) increased tumor infiltrating lymphocytes (TILs), (2) increased T cell diversity, (3) increased T cell clonality, (4) increased thymocytes, (5) increased thymus size, (6) thymic epithelial cells, (7) thymic stromal cells, or a combination thereof, compared to the subject before administering.

26. The method of claim 21, wherein the agent is a cytokine, a fusion molecule, or a ribonucleic acid (RNA) molecule.

27. The method of claim 26, wherein the cytokine is interleukin 7 (IL-7) or derivative thereof.

28. The method of claim 26, wherein the fusion molecule comprises an interleukin 7 (IL-7) molecule or a fragment thereof fused to an additional polypeptide or a polyethylene glycol (PEG) molecule.

29. The method of claim 28, wherein the additional polypeptide is an interleukin 15 (IL-15) molecule or a fragment thereof, an interleukin 12 (IL-12) molecule or a fragment thereof, an HGF beta molecule or a fragment thereof, an Fc domain of an immunoglobulin molecule, or a peptide having a sequence identity of at least 70% to SEQ ID NO: 14 or 15.

30. The method of claim 26, wherein the RNA molecule is fully or partially complementary to a sequence of a target nucleic acid molecule in the subject.

31. The method of claim 26, wherein the RNA molecule is a mRNA molecule encoding IL-7 or a fragment thereof, CD127 or a fragment thereof, soluble IL-7Rα or a fragment thereof, a CD127 activating monoclonal antibody, an anti-IL-7 antibody, IL-12 or a fragment thereof, IL-22 or a fragment thereof, IL-23 or a fragment thereof, KGF or a fragment thereof, GFG21 or a fragment thereof, Flt3L or a fragment thereof, IGF-1 or a fragment thereof, Ghrelin/GH or a fragment thereof, BMP-4 or a fragment thereof, IL-15 or a fragment thereof, a hormone, a GnRH antagonist, a GnRH agonist, sex steroid ablation, an aromatase inhibitor, an estrogen receptor agonist or antagonist, or any combination thereof.

32. The method of claim 21, wherein the inhibitor of immune checkpoint comprises a chimeric antigen receptor, wherein persistence and/or function of the chimeric antigen receptor is increased in the subject.

33. The method of claim 21, wherein the inhibitor of immune checkpoint is an inhibitor of B7-H3, B7-H4, BTLA, HVEM, TIM3, GALS, LAG3, VISTA, KIR, 2B4, CD160, CGEN-15049, CHK 1, CHK2, A2aR, B-7 family ligands, or a combination thereof.

34. The method of claim 21, further comprising, subsequent to administering, assessing one or more markers for T cell exhaustion in the subject.

35. A composition comprising (i) an agent that modulates thymus or T cell function and (ii) an inhibitor of immune checkpoint, wherein said composition is capable of decreasing T-cell exhaustion in a subject in need thereof, and wherein the subject has an expression of a cytokine receptor chain in a Tumor-infiltrating Lymphocyte (TIL) at least 10% higher than that of a non-memory effector cell from a healthy control, wherein the cytokine receptor chain is CD127, CD25, CD122, CD124, CD360 or CD132.

36. The composition of claim 35, further comprising, prior to administering, assessing one or more markers for T cell exhaustion in the subject.

37. The composition of claim 36, wherein the one or more markers comprise one or more transcription factors selected from: eomesodermin, T-bet, GATA-3, BCL-6, Helios, NFAT, Blimp-1, FoxO1, c-myc, or a combination thereof.

38. The composition of claim 36, wherein the one or more markers comprise one or more cytokines selected from: IL-7, IL-2, IL-4, IL-9, IL-15, IL-21, and a combination thereof, and wherein an expression level of the one or more cytokines of a tumor biopsy from the subject is lower than that of a biopsy from a healthy tissue.

39. The composition of claim 35, wherein, after administering, the subject has (1) increased tumor infiltrating lymphocytes (TILs), (2) increased T cell diversity, (3) increased T cell clonality, (4) increased thymocytes, (5) increased thymus size, (6) thymic epithelial cells, (7) thymic stromal cells, or a combination thereof, compared to the subject before administering.

40. The composition of claim 35, wherein the agent is a cytokine, a fusion molecule, or a ribonucleic acid (RNA) molecule.

41. The composition of claim 40, wherein the cytokine is interleukin 7 (IL-7) or derivative thereof

42. The composition of claim 40, wherein the fusion molecule comprises an interleukin 7 (IL-7) molecule or a fragment thereof fused to an additional polypeptide or a polyethylene glycol (PEG) molecule.

43. The composition of claim 42, wherein the additional polypeptide is an interleukin 15 (IL-15) molecule or a fragment thereof, an interleukin 12 (IL-12) molecule or a fragment thereof, an HGF beta molecule or a fragment thereof, an Fc domain of an immunoglobulin molecule, or a peptide having a sequence identity of at least 70% to SEQ ID NO: 14 or 15.

44. The composition of claim 40, wherein the RNA molecule is fully or partially complementary to a sequence of a target nucleic acid molecule in the subject.

45. The composition of claim 40, wherein the RNA molecule is a mRNA molecule encoding IL-7 or a fragment thereof, CD127 or a fragment thereof, soluble IL-7Rα or a fragment thereof, a CD127 activating monoclonal antibody, an anti-IL-7 antibody, IL-12 or a fragment thereof, IL-22 or a fragment thereof, IL-23 or a fragment thereof, KGF or a fragment thereof, GFG21 or a fragment thereof, Flt3L or a fragment thereof, IGF-1 or a fragment thereof, Ghrelin/GH or a fragment thereof, BMP-4 or a fragment thereof, IL-15 or a fragment thereof, a hormone, a GnRH antagonist, a GnRH agonist, sex steroid ablation, an aromatase inhibitor, an estrogen receptor agonist or antagonist, or any combination thereof.

46. The composition of claim 35, wherein the inhibitor of immune checkpoint comprises a chimeric antigen receptor, wherein persistence and/or function of the chimeric antigen receptor is increased in the subject.

47. The composition of claim 35, wherein the inhibitor of immune checkpoint is an inhibitor of B7-H3, B7-H4, BTLA, HVEM, TIM3, GALS, LAG3, VISTA, KIR, 2B4, CD160, CGEN-15049, CHK 1, CHK2, A2aR, B-7 family ligands, or a combination thereof.
